(19) United States
(12) Patent Application Publication
(10) Pub. No.: US 2022/0218741 A1
(43) Pub. Date: Jul. 14, 2022

(54) Cell-Mediated Synthesis of Noble Metal Oxide Nanoparticles and Biomedical Applications Thereof

(71) Applicant: Northeastern University, Boston, MA (US)

(72) Inventors: David Medina CRUZ, Jamaica Plain, MA (US); Junjiang CHEN, Boston, MA (US); Thomas J. WEBSTER, Barrington, RI (US)

(21) Appl. No.: 17/613,157

(22) PCT Filed: Jun. 1, 2020
(86) PCT No.: PCT/US2020/035617

§ 371 Date: Nov. 22, 2021

Related U.S. Application Data
(60) Provisional application No. 62/855,888, filed on May 31, 2019.

(51) Int. Cl. A61K 33/242; A61K 33/24; A61K 33/243; A61K 33/38; A61P 35/00; C12P 3/00; A61K 9/51

(52) U.S. Cl. CPC A61K 33/242; A61K 33/24; A61K 33/243; B82Y 5/00; A61P 35/00; C12P 3/00; A61K 9/5115; A61K 33/38

(57) ABSTRACT

Human dermal fibroblasts (HDF) and melanoma (MEL) cells are used herein for synthesis of metal nanoparticles. For example, synthesis of nanoparticles of gold (Au), palladium (Pd), platinum (Pt), and bimetallic formulations of gold-palladium (AuPd) and gold-platinum (AuPt) is demonstrated with HDF and MEL using a straightforward, eco-friendly and cost-effective approach. The nanostructures are purified and used in biomedical tests, which show selective behavior. The production of nanoparticles allows for stopping of the growth of cancer cells and the ability of new healthy cells to grow on top. The production of nanoparticles with the cells allows for an environmental-resistance behavior within the cells, showing the ability to stand for extreme environmental conditions.

### CROSS-REFERENCE TO RELATED APPLICATIONS

This application claims priority to U.S. Provisional Application No. 62/855,888, filed 31 May 2019, the entirety of which is incorporated herein by reference.

### BACKGROUND

Treatments for cancer include chemotherapy, surgery, and radiotherapy. Chemotherapy can significantly impact tumor growth; however, while the appropriate dosage of drugs can affect the tumor, it can also damage healthy tissue. Radiotherapy, which is often combined with surgery, can kill or delay the growth of cancer cells by destroying their DNA after exposure to radiation. Nonetheless, radiotherapy can cause adverse side effects to tissues near the targeted area. The conventional treatments of surgery, chemotherapy, and radiotherapy are associated with significant negative side effects, which calls for alternative treatments.

Nanotechnology in medicine, known as nanomedicine, can bring doctors and patients new opportunities for improved cancer treatments. Since nanoparticles are hundreds of times smaller than human cells and can interact with cells, they may provide a suitable solution to the problems associated with current cancer treatments. Selective targeting abilities and higher cell permeability of nanostructures, together with the potential for in vivo tracking and wide tenability, allowing for easier control of size, shape, and composition, leading to different biocompatibility and biodistribution features, provide opportunities in nanomedicine. Thus, new methods and compositions for treating cancer utilizing nanoparticles are urgently needed.

### SUMMARY

Green chemistry methods for synthesis of metallic nanoparticles are provided herein. For example, gold (Au), palladium (Pd), platinum (Pt), bimetallic gold-palladium (AuPd), and gold-platinum (AuPt) nanoparticles can be synthesized intracellularly and extracellularly in different human living cell lines (cancer and healthy cells) through reduction of ions. Extensive characterizations in terms of morphology, composition, and surface chemistry through TEM, SEM, XRD, and UV-Vis absorption techniques are shown to demonstrate the formation of noble metal nanoparticles inside different compartments of the cells, as well as larger particles of different sizes and shapes in the incubation solution. The effects of the precursor metal ions on cell viability as well as cell morphology in different living cell lines are shown. The results demonstrate that treatment of different cell lines with metal ions results in the cell fixation for a mechanism that is investigated for first time.

The present technology can be further summarized by the following features.

1. A method of inhibiting the growth of cancer cells in a subject, the method comprising administering a therapeutically effective amount of coated metal nanoparticles to the subject, whereby the growth of the cancer cells in the subject is inhibited;

wherein the metal nanoparticles are produced by a process comprising growing human cells in the presence of a metal salt, whereby metal ions of the salt are reduced to elemental metal to form the metal nanoparticles; whereby the human cells deposit a coating of organic molecules on the metal nanoparticles; and

wherein the coated metal nanoparticles selectively inhibit growth of the cancer cells compared to inhibition by the coated metal nanoparticles of growth of non-cancerous cells in the subject.

2. The method of claim 1, further comprising, prior to said administering:

collecting a sample of the cancer cells and a sample of normal cells from the subject;

cultivating the cancer cells and the normal cells in vitro; and

forming said coated metal nanoparticles by growing the cultivated normal cells in the presence of said metal salt, whereby metal ions of the metal salt are reduced to elemental metal to form said metal nanoparticles.

3. The method of any of the preceding claims, wherein the coated metal nanoparticles are at least partially coated with organic molecules provided by the human cells.
4. The method of any of the preceding claims, wherein a minimum inhibitory concentration of the coated metal nanoparticles for the cancer cells is in the range from about 5 to 50 μg/mL.
5. The method of any of the preceding claims, wherein an IC₅₀ for growth inhibition of the cancer cells is from about 30 to about 65 μg/mL.
6. The method of any of the preceding claims, wherein the coated metal nanoparticles have a zeta potential in the range from about 30 mV to about 50 mV.
7. The method of any of the preceding claims, wherein the administered coated metal nanoparticles are formulated with one or more pharmaceutically acceptable excipients.
8. The method of any of the preceding claims, wherein said coated metal nanoparticles comprise a metal oxide.
9. The method of any of the preceding claims, wherein the human cells are selected from human dermal fibroblasts and human melanoma cells
10. The method of any of the preceding claims, wherein the coating inhibits the growth of cancer cells.
11. The method of any of the preceding claims, wherein the coated metal nanoparticles comprise Au, Ag, Se, Te, ZnO, CuO, Fe₂O₃, Fe₃O₄, Pt, Pd, or a combination thereof.
12. The method of any of the preceding claims, wherein the metal salt is selected from the group consisting of HAuCl₄, K₂PtCl₄, K₂PdCl₄, and mixtures thereof.
13. The method of any of the preceding claims, wherein the coated metal nanoparticles comprise a radioisotope.
14. The method of any of the preceding claims, wherein the coated metal nanoparticles possess a magnetic property.
15. The method of any of the preceding claims, wherein the coated metal nanoparticles further comprise a moiety selected from the group consisting of a protein, an antibody, an oligonucleotide, and a small molecule drug.
16. The method of any of the preceding claims, wherein the coating is a targeting moiety capable of targeting the coated metal nanoparticles to the cancer cells.
17. The method of any of the preceding claims, wherein the cancer cells are cells of a cancer selected from the group consisting of skin cancer, lung cancer, breast cancer, prostate cancer, colorectal cancer, bladder cancer, melanoma, Non-Hodgkin lymphoma, kidney cancer, and leukemia.
18. The method of any of the preceding claims, wherein the growth of non-cancerous cells in the subject is not substantially inhibited.
19. The method of any of the preceding claims, wherein the therapeutically effective amount provides a concentration of coated metal nanoparticles of about 25 μg/mL at or near the cancer cells.
20. The method of any of the preceding claims, wherein the coated metal nanoparticles cause a lethal increase in reactive oxygen species in the cancer cells.
21. The method of any of the preceding claims, wherein a portion of the metal nanoparticles is synthesized in the cytoplasm of the human cell.
22. Coated metal nanoparticles produced by a process comprising growing a first type of human cell in the presence of a metal salt, wherein metal ions of the salt are reduced to elemental metal and the first type of human cell deposits a coating of organic molecules on the elemental metal, wherein the coated metal nanoparticles are capable of selectively inhibiting growth of a second type of human cell more than the coated metal nanoparticles inhibit growth of the first type of human cell.
23. The method of claim 22, wherein the coated metal nanoparticles are at least partially coated with organic molecules provided by the first type of human cell during the process of producing the coated metal nanoparticle.
24. The method of claim 22, wherein the organic coating causes the coated metal nanoparticles to selectively inhibit growth of the second type of human cell compared to other types of human cells.
25. The method of claim 22, wherein the organic coating comprises one or more biomolecules specific to the first type of human cells.
26. The method of claim 22, wherein the coated metal nanoparticles further comprise a moiety selected from the group consisting of a radioisotope, a protein, an antibody, an oligonucleotide, a small molecule, and a therapeutic agent.
27. The method of claim 22, wherein the nanoparticles have an average diameter in the range from about 1 nm to about 30 nm, or about 5 to about 25 nm.
28. The method of claim 22, wherein the organic coating is operative to stabilize the coated metal nanoparticles as a colloid or suspension for at least about 60 days.
29. The method of claim 22, wherein the organic coating provides the nanoparticles with a zeta potential exceeding +30 mV which is stable for at least about 60 days.
30. The method of claim 22, wherein the atomic structure of the metal comprises amorphous, FCC, or a combination thereof.
31. The method of claim 22, wherein the coated metal nanoparticles comprise a metal oxide.
32. A method of inhibiting growth of a cancer cell, the method comprising contacting the cancer cell with the coated metal nanoparticles of any of claims 22 to 30, wherein the contacting is performed by administering the coated metal nanostructures to a subject having a cancer, and wherein proliferation of a cancer cell in the subject is inhibited but proliferation of normal cells of the subject is not significantly inhibited.
33. A method of producing coated metal nanoparticles, the method comprising:

(a) contacting a first type of human cell with a metal salt; and

(b) allowing the first type of human cell to reduce the metal salt to elemental metal and to deposit an organic coating on the elemental metal;

whereby coated metal nanoparticles are produced.
34. The method of claim 33, further comprising:

(c) centrifuging the product resulting from step (b) to obtain a pellet;

(d) resuspending the pellet in water; and

(e) lyophilizing the resuspended pellet.

35. The method of claim 33 or 34, wherein the resulting coated metal nanoparticles each have a diameter of about 15 nm to about 35 nm.
36. The method of any of claims 33-35, wherein the temperature in step (b) is in the range from about 20° C. to about 40° C.
37. The method of any of claims 33-36, wherein the atomic structure of the coated metal nanoparticles comprise amorphous metal, FCC metal, or a combination thereof.
38. The method of any of claims 33-37, wherein the method produces no byproducts toxic to normal human cells.
39. The method of any of claims 33-38, wherein the first type of human cell is a human dermal fibroblast cell or a human melanoma cell.
40. The method of any of claims 33-39, wherein the elemental metal or metal oxide is Au, Ag, Se, Te, ZnO, CuO, Fe₂O₃, Fe₃O₄, Pt, Pd, or a combination thereof.
41. The method of any of claims 33-40, wherein the metal salt is selected from the group consisting of HAuCl₄, K₂PtCl₄, K₂PdCl₄, and mixtures thereof.

As used herein, minimum inhibitory concentration (MIC) is the lowest concentration of a coated metal nanoparticle that will inhibit, in vitro, the visible growth of a cell or microorganism after 24 hours of incubation. The half maximal inhibitory concentration (IC₅₀) is the concentration of a coated metal nanoparticle that is needed to inhibit, in vitro, the growth of a cell or microorganism by 50%. The chemical “MTS” utilized in MTS assays described herein refers to MTS (3-(4,5-dimethylthiazol-2-yl)-5-(3-carboxymethoxyphenyl)-2-(4-sulfophenyl)-2H-tetrazolium).

As used herein, metal nanoparticles refers to nanoparticles comprising metals, metalloids, metal oxides, and combinations thereof.

As used herein, the term “about” and “approximately” are defined to be within 10%, 5%, 1%, or 0.5% of the stated value. As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

### BRIEF DESCRIPTION OF THE DRAWINGS

FIGS. 1A-1E show UV-vis-NIR spectra (units of absorbance, u.a. vs. ˜250-800 nm) of the supernatant and lysed solution of HDF (human dermal fibroblast) cells incubated with HAuCl₄(A), K₂PdCl₄(B), K₂PtCl₄(C), HAuCl₄ and K₂PdCl₄(D), HAuCl₄ and K₂PtCl₄(E) in DPBS for different times (time 0 hours=▪, 6 hours=●, 12 hours=▴, 24 hours=▾, lysis=♦).

FIGS. 2A-2E show UV-vis-NIR spectra of the supernatant and lysed solution of melanoma cells incubated with HAuCl₄(A), K₂PdCl₄(B), K₂PtCl₄(C), HAuCl₄ and K₂PdCl₄(D), HAuCl₄ and K₂PtCl₄(E) in DPBS for different times (time 0 hours=▪, 6 hours=●, 12 hours=▴, 24 hours=▾, lysis=♦).

FIGS. 3A-3F show light microscopy images of HDF cells right after incubation with HAuCl₄(A), K₂PtCl₄(B), K₂PdCl₄(C), HAuCl₄ and K₂PtCl₄(D), HAuCl₄ and K₂PdCl₄(E) in DPBS, and control is incubation with DPBS (F).

FIGS. 4A-4F show microscopy images of HDF cells 72 hours after incubation with HAuCl₄(A), K₂PtCl₄(B), K₂PdCl₄(C), HAuCl₄ and K₂PtCl₄(D), HAuCl₄ and K₂PdCl₄(E) in DPBS, and control is incubation with DPBS (F).

FIGS. 5A-5F show microscopy images of melanoma cells right after inoculation with HAuCl₄(A), K₂PtCl₄(B), K₂PdCl₄(C), HAuCl₄ and K₂PtCl₄(D), HAuCl₄ and K₂PdCl₄(E) in DPBS, and control is incubation with DPBS (F).

FIGS. 6A-6F show microscopy images of melanoma cells 72 hours after incubation with HAuCl₄(A), K₂PtCl₄(B), K₂PdCl₄(C), HAuCl₄ and K₂PtCl₄(D), HAuCl₄ and K₂PdCl₄(E) in DPBS, and control is incubation with DPBS (F).

FIGS. 7A-7E show TEM images of Au(A), Pt(B), Pd(C), AuPt(D), and AuPd(E) NPs (nanoparticles) after purification, biosynthesized from HDF cells.

FIGS. 8A-8E show TEM images of Au(A), Pt(B), Pd(C), AuPt(D), AuPd(E) NPs after purification, biosynthesized from melanoma cells.

FIG. 9 shows (overlay) comparison between the experimental XRD patterns for (A, top trace) HDF-AuNPs, (B, 2^{nd} from top trace) HDF-PtNPs, (C, 3^{rd} from top trace) HDF-PdNPs, (D, 4^{th} from top trace) HDF-AuPtNPs, (E, 2^{nd} from bottom trace) HDF-AuPdNPs, and the calculated XRD pattern for cubic PdO (F, bottom trace).

FIG. 10 shows comparison between the experimental XRD patterns for (A, top trace) MEL-AuNPs, (B, 2^{nd} from top trace) MEL-PtNPs, (C, 3^{rd} from top trace) MEL-PdNPs, (D, 4^{th} from top trace) MEL-AuPtNPs, (E, 3^{rd} from bottom trace) MEL-AuPdNPs, and the calculated XRD patterns for (F, 2^{nd} from bottom trace) cubic PdO, and (G, bottom trace) FCC Au.

FIGS. 11A-11F show SEM images of HDF cells after 24 hours incubation with only DPBS (A), HAuCl₄(B), K₂PdCl₄(C), K₂PtCl₄(D), HAuCl₄ and K₂PdCl₄(E), HAuCl₄ and K₂PtCl₄(F) in DPBS; all at lower magnification.

FIGS. 12A-12F show SEM images of melanoma cells after 24 hours incubation with only DPBS(A), HAuCl₄(B), K₂PdCl₄(C), K₂PtCl₄(D), HAuCl₄ and K₂PdCl₄(E), HAuCl₄ and K₂PtCl₄(F) in DPBS; all at lower magnification.

FIGS. 13A-13F show SEM images of HDF cells after 24 hours incubation with only DPBS(A), HAuCl₄(B), K₂PdCl₄(C), K₂PtCl₄(D), HAuCl₄ and K₂PdCl₄(E), HAuCl₄ and K₂PtCl₄(F) in DPBS; all shown at higher magnifications.

FIGS. 14A-14F show SEM images of melanoma cells after 24 hours incubation with only DPBS(A), HAuCl₄(B), K₂PdCl₄(C), K₂PtCl₄(D), HAuCl₄ and K₂PdCl₄(E), HAuCl₄ and K₂PtCl₄(F) in DPBS; all at higher magnifications.

FIGS. 15A-15E show MTS assays on HDF cells cultured in the presence of HDF-AuNPs (A), HDF-PdNPs (B), HDF-PtNPs (C), HDF-AuPdNPs (D), and HDF-AuPtNPs(E) ranging from 0 to 100 μg/mL (0, 25, 50, 75, 100 μg/mL). For each concentration (μg/mL), the left bar is 24 hours, and the right bar is 72 hours. The HDF prefix designates the nanoparticles were synthesized using HDF cells.

FIGS. 16A-16E show MTS assays on melanoma cells cultured in the presence of HDF-AuNPs (A), HDF-PdNPs (B), HDF-PtNPs (C), HDF-AuPdNPs (D), and HDF-AuPtNPs(E) ranging from 0 to 100 μg/mL (0, 25, 50, 75, 100 μg/mL). For each concentration (μg/mL), the left bar is 24 hours, and the right bar is 72 hours.

FIGS. 17A-17E show MTS assays on HDF cells cultured in the presence of MEL-AuNPs (A), MEL-PdNPs (B), MEL-PtNPs (C), MEL-AuPdNPs (D), and MEL-AuPtNPs(E) ranging from 0 to 100 μg/mL (0, 25, 50, 75, 100 μg/mL). For each concentration (μg/mL), the left bar is 24 hours, and the right bar is 72 hours. The MEL prefix designates the nanoparticles were synthesized using human melanoma cells.

FIGS. 18A-18E show MTS assays on melanoma cells cultured in the presence of MEL-AuNPs (A), MEL-PdNPs (B), MEL-PtNPs (C), MEL-AuPdNPs (D) and MEL-AuPtNPs(E) ranging from 0 to 100 μg/mL (0, 25, 50, 75, 100 μg/mL). For each concentration (μg/mL), the left bar is 24 hours, and the right bar is 72 hours.

FIGS. 19A-19F show SEM images of HDF cells after 24 hours incubation with only EMEM(A), HDF-AuNPs(B), HDF-PdNPs(C), HDF-PtNPs (D), HDF-AuPdNPs (E), HDF-AuPtNPs (F) in EMEM at higher (50 k) magnification. EMEM refers to Eagle's minimum essential medium.

FIGS. 20A-20F show SEM images of melanoma cells after 24 hours incubation with only DMEM(A), HDF-AuNPs(B), HDF-PdNPs(C), HDF-PtNPs (D), HDF-AuPdNPs (E), HDF-AuPtNPs (F) in DMEM at higher (50 k) magnification. DMEM refers to Dulbecco's modified eagle medium.

FIGS. 21A-21E show ROS (reactive oxygen species) studies of HDF-AuNPs(A), HDF-PdNPs(B), HDF-PtNPs(C), HDF-AuPdNPs(D), and HDF-AuPtNPs(E) against melanoma cells. Fluorescence intensity (% of control) is plotted (Y-axis) v. nanoparticle concentration (μg/mL, X-axis).

FIG. 22 shows MTS assays on melanoma cells incubated with new DMEM medium after different types of treatment with metallic solutions. The previously applied treatment types are labeled on the X-axis. For each treatment type on the X-axis, the left bar plotted is for 24 hours, and the right bar plotted is for 72 hours.

FIG. 23 shows cell proliferation (%) of melanoma cells (cultured 24 hours) in DMEM (5×10⁴ cells/well in 96 well size plates) after removal of media, adding DPBS to wash once, then adding metallic salt (Au, Pt, Pd, AuPt, AuPd, positive media control, or negative DPBS control, X-axis) with another 24 hours cultivation thereafter. After total 48 hours, the supernatant was removed and the cells divided into 3 groups: left bar=new DMEM media added, center bar=new HDF cells added (5×10⁴ cells/well), right bar=new melanoma cells added (5×10⁴ cells/well). The 3 groups were then cultured 24 hours. To measure final proliferation, the supernatant was removed, MTS:DMEM (1:5) was added, and final measurement after 4 hours.

FIG. 24 shows cell proliferation (%) of HDF cells (24 hours) in DMEM (5×10⁴ cells/well in 96 well size plates) after removal of media, adding DPBS to wash once, then adding metallic salt (Au, Pt, Pd, AuPt, AuPd, positive media control, or negative DPBS control, X-axis) with another 24 hours cultivation. After total 48 hours, the supernatant was removed and the cells divided into 3 groups: left bar=new DMEM media added, center bar=new HDF cells added (5×10⁴ cells/well), right bar=new melanoma cells added (5×10⁴ cells/well). The 3 groups were then cultured 24 hours. To measure final proliferation, the supernatant was removed, MTS:DMEM (1:5) was added, and final measurement after 4 hours.

FIGS. 25A-25L show microscopy images of different HDF cells at a highly acidic HCl environment (pH˜1). HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in acidic environment.

FIGS. 26A-26L show microscopy images of different melanoma cells at a highly acidic HCl environment (pH˜1). Melanoma cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal melanoma cells at 0(K) and 24 h(L) in acidic environment.

FIGS. 27A-27L show microscopy images of different HDF cells at a highly basic NaOH environment (pH˜13). HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in highly basic environment.

FIGS. 28A-28L show microscopy images of different melanoma cells at a highly basic NaOH environment (pH˜13). Melanoma cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in highly basic environment.

FIGS. 29A-29L show microscopy images of different HDF cells at a high concentration of NaCl (1 M) environment. HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in NaCl (1 M) environment.

FIGS. 30A-30L show microscopy images of different melanoma cells at a high concentration of NaCl (1 M) environment. Melanoma cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal melanoma cells at 0(K) and 24 h(L) in NaCl (1 M) environment.

FIGS. 31A-31L show microscopy images of different HDF cells in an only DI-water environment. HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in a DI-water environment.

FIGS. 32A-32L show microscopy images of different melanoma cells in an only DI-water environment. Melanoma cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal melanoma cells at 0(K) and 24 h(L) in DI-water environment.

FIGS. 33A-33L show microscopy images of different HDF cells at a high concentrated trypsin environment (0.5%). HDF cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and normal HDF cells at 0(K) and 72 h(L) in trypsin environment.

FIGS. 34A-34L show microscopy images of different melanoma cells at high concentrated trypsin environment (0.5%). Melanoma cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and normal melanoma cells at 0(K) and 72 h(L) in trypsin environment.

FIGS. 35A-35L show microscopy images of different HDF cells at high temperature environment (50° C.). HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in high temperature environment.

FIGS. 36A-36L show microscopy images of different melanoma cells at high temperature environment (50° C.). Melanoma cells treated HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal melanoma cells at 0(K) and 24 h(L) in high temperature environment.

FIGS. 37A-37L show microscopy images of different HDF cells at a low temperature environment (−80° C.). HDF cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal HDF cells at 0(K) and 24 h(L) in a low temperature environment.

FIGS. 38A-38L show microscopy images of different melanoma cells at a low temperature environment (−80° C.). Melanoma cells treated with HAuCl₄ at 0(A) and 24 h(B), K₂PtCl₄ at 0(C) and 24 h(D), K₂PdCl₄ at 0(E) and 24 h(F), HAuCl₄ and K₂PtCl₄ at 0(G) and 24 h(H), HAuCl₄ and K₂PdCl₄ at 0(I) and 24 h(J), and normal melanoma cells at 0(K) and 24 h(L) in a low temperature environment.

FIGS. 39A-39L show microscopy images of different HDF cells with a supernatant of treated HDF cells environment. HDF cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and only DPBS at 0(K) and 72 h(L) in the environment.

FIGS. 40A-40L show microscopy images of different HDF cells with a supernatant of treated melanoma cells environment. HDF cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and only DPBS at 0(K) and 72 h(L) in the supernatant of treated melanoma cells environment.

FIGS. 41A-41L show microscopy images of different melanoma cells with a supernatant of treated HDF environment. Melanoma cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and only DPBS at 0(K) and 72 h(L) in the environment.

FIGS. 42A-42L show microscopy images of different melanoma cells with a supernatant of treated melanoma environment. Melanoma cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and only DPBS at 0(K) and 72 h(L) in the supernatant of treated melanoma environment.

FIGS. 43A-43L show microscopy images of different HDF cells at new EMEM medium environment. HDF cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and normal HDF cells at 0(K) and 72 h(L) in new EMEM environment.

FIGS. 44A-44L show microscopy images of different melanoma cells at new DMEM environment. Melanoma cells treated with HAuCl₄ at 0(A) and 72 h(B), K₂PtCl₄ at 0(C) and 72 h(D), K₂PdCl₄ at 0(E) and 72 h(F), HAuCl₄ and K₂PtCl₄ at 0(G) and 72 h(H), HAuCl₄ and K₂PdCl₄ at 0(I) and 72 h(J), and normal melanoma cells at 0(K) and 72 h(L) in new DMEM environment.

FIGS. 45A-45L show microscopy images of treated melanoma cells before (A, C, E, G, 1, K) and 72 hours (B, D, F, H, J, L) after adding new HDF cells in EMEM environment. Treatment types: HAuCl₄ (A, B), K₂PtCl₄ (C, D), K₂PdCl₄ (E, F), HAuCl₄ and K₂PtCl₄ (G, H), HAuCl₄ and K₂PdCl₄ (1, J), and only DPBS (K, L).

### DETAILED DESCRIPTION

Human dermal fibroblasts (HDF) and melanoma (MEL) cells are used herein for synthesis of metal nanoparticles. For example, synthesis of nanoparticles of gold (Au), palladium (Pd), platinum (Pt), and bimetallic formulations of gold-palladium (AuPd) and gold-platinum (AuPt) is demonstrated with HDF and MEL using a straightforward, eco-friendly and cost-effective approach. The nanostructures are purified and used in biomedical tests, which show selective behavior. The production of nanoparticles with the cells allows for an environmental-resistance behavior within the cells, showing the ability to stand for extreme environmental conditions. The production of nanoparticles allows for stopping of the growth of cancer cells and the ability of new healthy cells to grow on top.

After purification and characterization the nanoparticles are used as biomedical agents in cytotoxicity studies. The nanoparticles show an interesting dose-dependent concentration selectivity towards different cell lines that might be related to the presence of particular molecules in the coating surrounding the nanoparticles whose origin is ligated to the cell that synthesizes it. It is possible to observe how HDF-synthesized nanoparticles show a strong anticancer effect, while no significant cytotoxicity effect was found towards HDF cells, with a converse behavior observed for nanoparticles synthesized with melanoma (MEL), in a range of concentrations between about 25 μg/mL and 100 μg/mL.

Upon culturing HDF cell with different metal solutions, visible color changes in the wells containing HDF cells and different metal solutions are sometimes observed either immediately after addition of the metal solution (e.g., metal salt solution) or after about 1-5 days. Similarly, when melanoma cells are cultured with different metal solutions or metal salt containing solutions, sometime color changes can be observed either immediately or after days of cultivation. It is hypothesized that the color changes of the solutions is due to the intracellular/extracellular synthesis of metallic nanoparticles. This process is carried out by the cells as a way to cope with highly toxic concentrations of metallic salts within the media. Nevertheless, the mechanism behind this transformation from metallic ions (or solution metal) to elemental nanostructures by living human cells is not completely understood, yet is due to the diversity and the different potential reduction agents, such as the cell membrane enzymes and other biomolecules present in the cytoplasm. Therefore, because of the complexity of the eukaryotic biological system, multiple factors can have credit for the reduction of metal ions.

The nanomaterials can be synthesized either inside or outside the cell membranes, and once released, they can be used for various biomedical and clinical applications, showing a higher biocompatibility and less toxicity for the biological tissue, together with enhanced surface areas that enables for a highly reactive area.

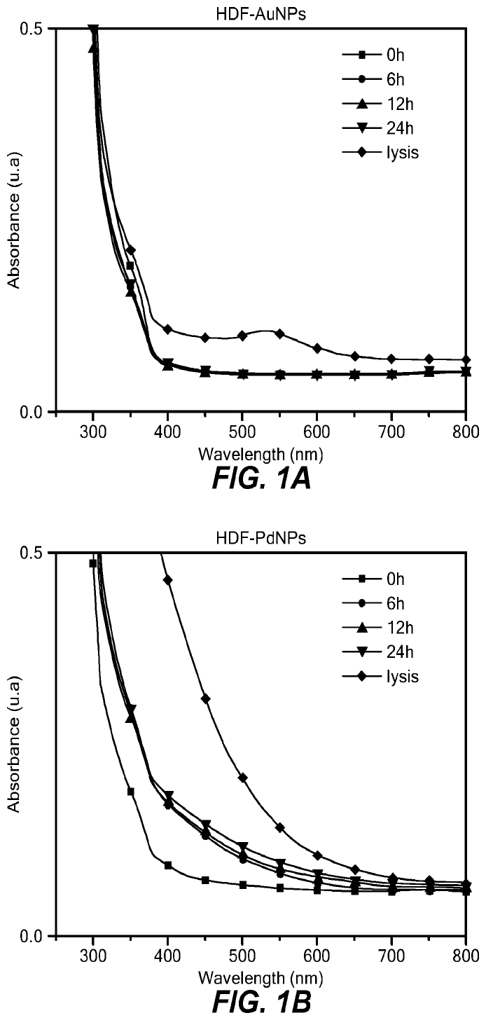

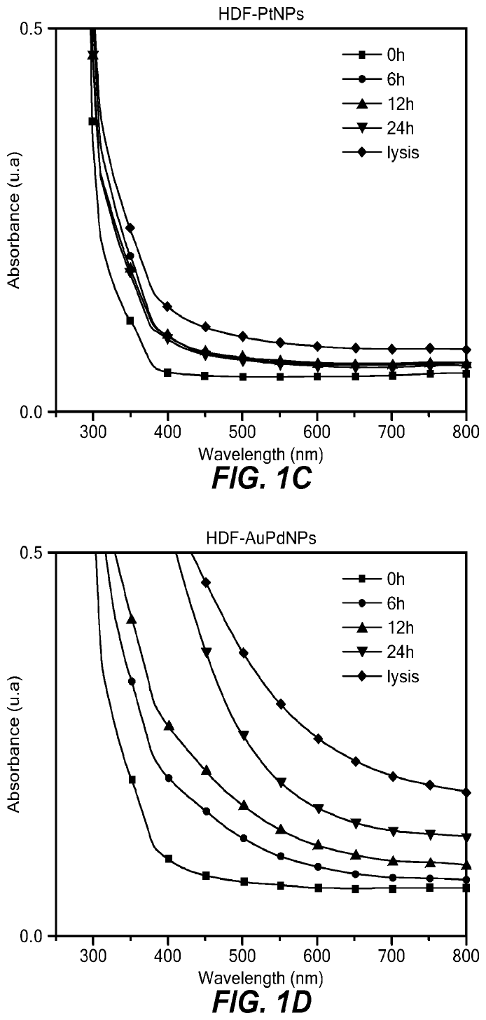

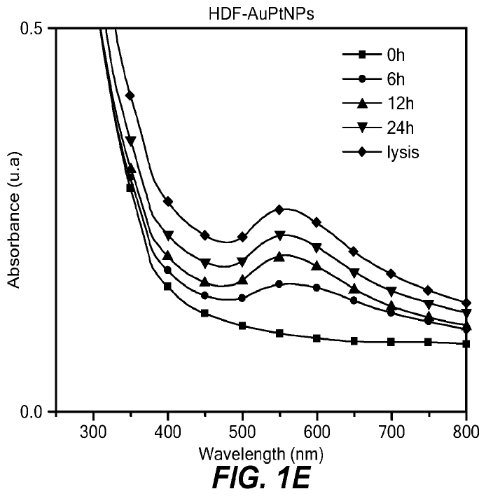

During synthesis, UV-visible absorption analyses (UV-Vis, 250-800 nm) were carried out to periodically measure the extracellular and lysate absorbance, monitoring the reduction of metallic ions over time. For HDF cells treated with HAuCl₄ solutions, FIG. 1A shows no increase in the absorbance 0-24 hours before lysis, then there is an increase in the absorbance at about 550 nm when the HDF cells were lysed. This observation demonstrates the AuNPs concentrations in the extracellular solution are consistent during the reduction process. This fact suggests that most of the AuNPs were inside the cells during the synthesis process and were released after lysis. Thus, this fact can be related to the fact that the observable color of the culture did not significantly change within 24 hours. For HDF cells treated with K₂PdCl₄ (FIG. 1B) and K₂PtCl₄ (FIG. 1C), the absorbance spectra before 24 hours are consistent but higher than 0 hours, which suggests that HDF-PdNPs and HDF-PtNPs were first released to the extracellular surroundings. For HDF cells cultured with combination Au and Pd salt solutions (FIG. 1D), a steady increase in absorbance is shown from 0 to 24 hours, which indicates the steady release of biosynthesized HDF-AuPdNPs. Moreover, for HDF cells treated with Au and Pt solutions (FIG. 1E), the absorbances steadily increase and the surface plasmon resonance band appears at about 550 nm. The 550 nm band resonance broadens and shifts, which indicates the increase of particle size and changes within the composition.

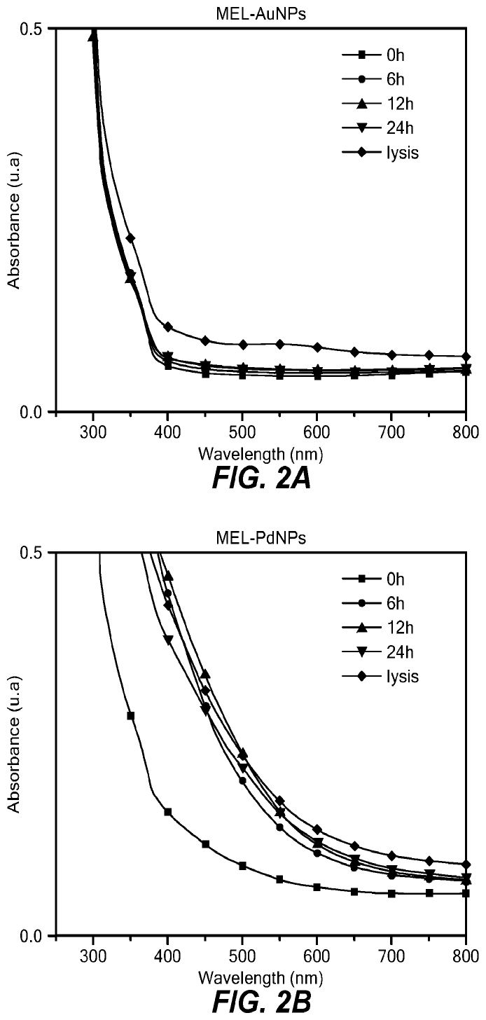

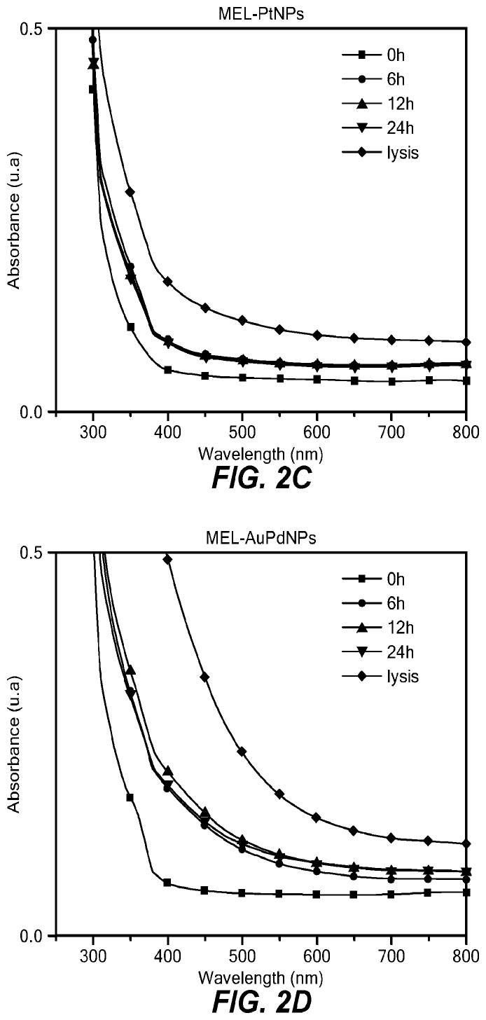

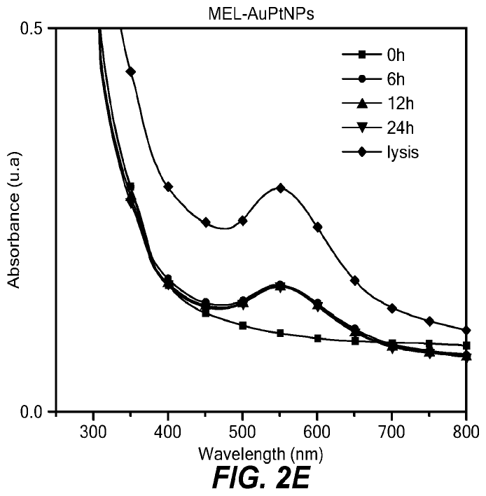

As shown in FIG. 2A, the absorbances of the melanoma cells treated with Au salt solutions (A) are consistent before lysis. Then the resonance band appears around 550 nm after lysis. The absorbances of the melanoma cells treated with Pd (FIG. 2B) and Pt (FIG. 2C) solutions first increase and then are constant before lysis, while the absorbances of melanoma cells treated with Au and Pd solutions (FIG. 2D) constantly increase, which indicates that melanoma cells allowed AuPdNPs to be first released to the extracellular media. This behavior might be caused by a quick release of nanoparticles by the melanoma cells, with a higher speed rate than the one found in HDF cells. For melanoma cells treated with Au and Pt solution (FIG. 2E), the absorbances are constant, while the lysed band at about 550 nm broadens and shifts, which indicate the particle size in cells are larger than the ones in solution.

The obtained results are related to the fact that the nanoparticles may be synthesized on the cell membrane surface. Moreover, it can be suggested that the nanoparticles are transferred from cytoplasm to the solution during the process, which is a reason why the UV-visible signatures grow continuously before lysis for most of the experiments.

Cell morphology and proliferation are studied using a light microscope. It can be seen from the figures that when the cells were incubated with metal solutions in DPBS, with no nutrients or media left, they stopped growing after a few minutes, leading to an irreversible cell fixation to the bottom of the plates. With the increase of incubation time, the cell color became darker which indicates the presence of clusters of metallic nanoparticles.

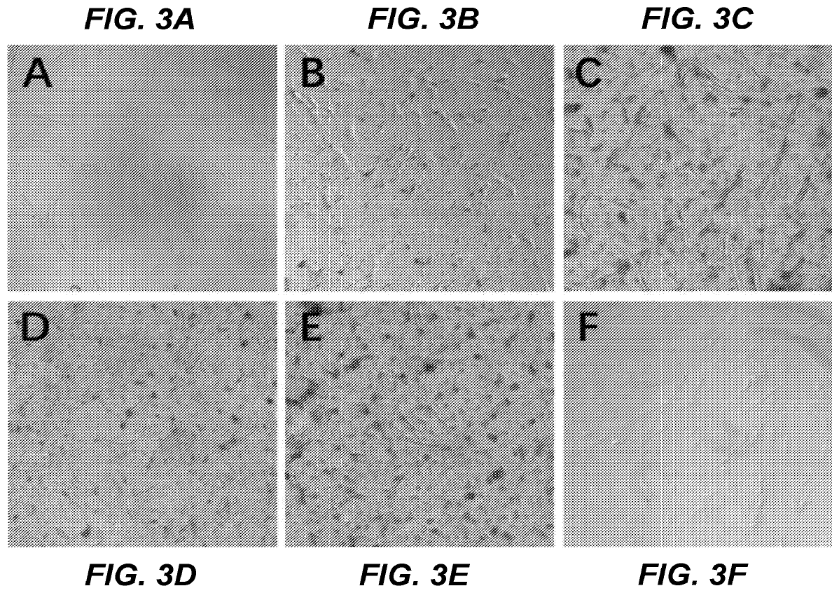

As it can be seen in FIGS. 3A-3F, right after the addition of metallic salts in DPBS (and just DPBS for the control in FIG. 3F) the cells remain attached to the bottom with their original morphology. The quick appearance of metallic nanoparticles can be found when the salts of Pt (FIG. 3B) and Pd (FIG. 3C) are added to the cells, leading to the observation of dark clusters all over the cell media (also FIGS. 3D and 3E). FIGS. 3A-3F show the microscopy images of HDF cells right after incubation with HAuCl₄(A), K₂PtCl₄(B), K₂PdCl₄(C), HAuCl₄ and K₂PtCl₄(D), HAuCl₄ and K₂PdCl₄(E) in DPBS and control incubation with DPBS (F).

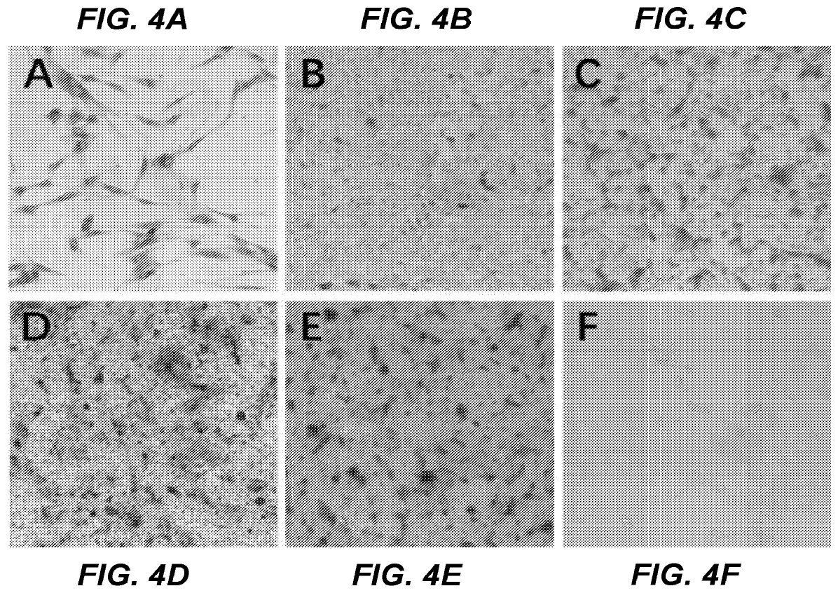

After 72 hours of experiment (FIG. 4A-4F), the cells that were incubated with different metallic salts remain attached to the bottom of the plates, keeping their original morphology and with no apparent shrinking or deformation. This observation is in clear contrast with the cells in the control (FIG. 4F), whose membrane shrinks and is subjected to normal deformation, leading to a detachment from the bottom and subsequent death due to the lack of nutrients in the media. FIGS. 4A-4F show microscopy images of HDF cells 72 hours after incubation with HAuCl₄(A), K₂PtCl₄(B), K₂PdCl₄(C), HAuCl₄ and K₂PtCl₄(D), HAuCl₄ and K₂PdCl₄(E) in DPBS, with the control incubation in DPBS shown in FIG. 4F.

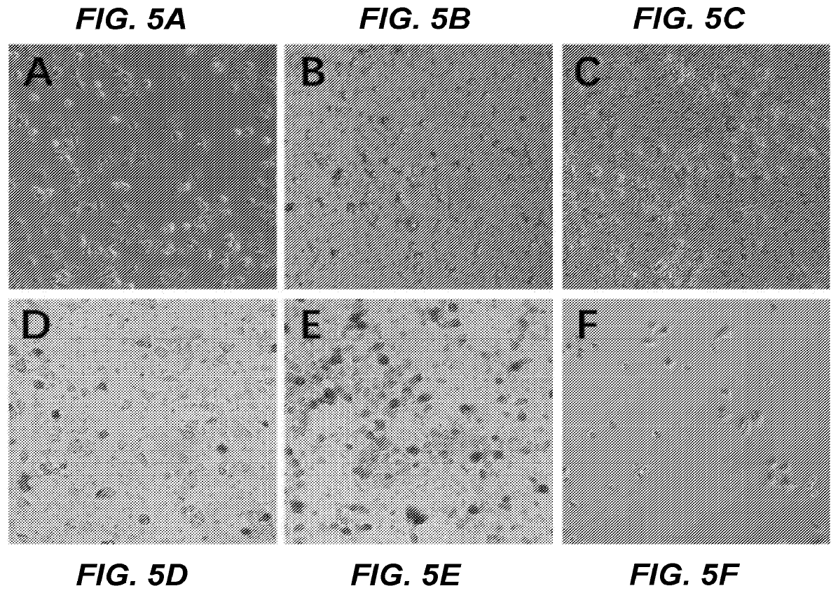

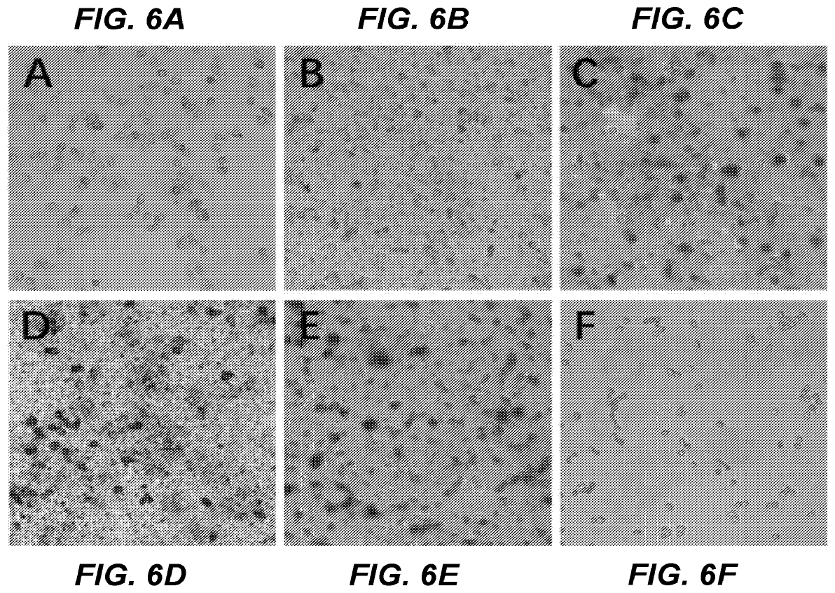

A light microscopy study of melanoma cells right after (FIGS. 5A-5F) and 72 hours (FIGS. 6A-6F) after the addition of Au (A), Pt (B), Pd (C), AuPt (D) and AuPd (E), together with a control of the cells in DPBS (F) is shown for comparison to HDF cells. As can be seen in FIGS. 5A-5F, right after the addition of metallic salts in DPBS (and just DPBS for the control in FIG. 5F) melanoma cells remain attached to the bottom with their original morphology. The quick appearance of metallic nanoparticles is found when the salts of Pt (FIG. 5B) and Pd (FIG. 5C) are added to the cells, leading to the observation of dark clusters all over the cell media, with dark clusters also observed in FIGS. 5D and 5E. These results are in accordance with the results found for HDF cells.

In FIGS. 6A-6F, after 72 hours of experiment, the cells that were incubated with different metallic salts remain attached to the bottom of the plates, keeping their original morphology and with no apparent shrinking or deformation. This observation is in clear contrast with the cells in the control (FIG. 6F), whose membrane shrinks and is subjected to normal deformation, leading to a detachment from the bottom and subsequent death due to the lack of nutrients in the media.

The results obtained from this first line of experiments suggest that both HDF and melanoma cells have a similar response to the presence of metallic salts, leading to a production of nanoparticles that is related to the fixation of the cells to the bottom. Both controls, cultured in DPBS, remained dead and detached, which clearly indicates that the findings are related to the production of nanoparticles, in a process that works in the same way for both cell lines.

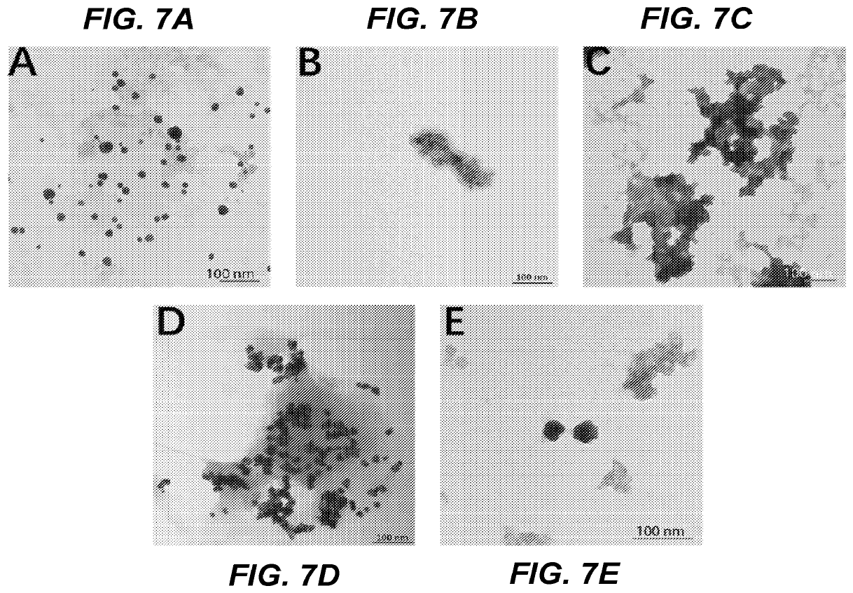

FIGS. 7A-7E show TEM images of Au(A), Pt(B), Pd(C), AuPt(D), and AuPd(E) NPs (nanoparticles) after purification, biosynthesized from HDF cells. As shown in FIGS. 7A-7E, TEM characterization shows that nanoparticles are successfully biosynthesized by HDF cells. After purification, the nanoparticles are removed from the cells and remain monodispersed in solution, with a size distribution below about 30 nm and surrounded with organic materials coming from the cells. The presence of these organic materials attached to the nanoparticles might be related to an intracellular synthesis. A complete set of size distribution values is summarized in Table 1. It can be seen from the TEM images that PtNPs (FIG. 7B) and PtNPs (FIG. 7C) appear as amorphous structures that only reach a defined structure when combined with gold (FIGS. 7D-7E). The size of the HDF-NPs can be in the range from about 1 nm to about 30 nm or 35 nm, from about 5 nm to about 25 nm, from about 15 nm to about 35 nm, from about 5 nm to about 20 nm, and from about 8 nm to about 18 nm. The size can be an average size, determined by average equivalent volume methods (in which the average is volume weighted) or by numerical methods, in which the average is numerically weighted.

TABLE 1
Size distribution of HDF-Au, -Pt, -Pd, -AuPt and -AuPdNPs

| Nanostructure | Diameter (nm) |
|---|---|
| HDF-AuNPs | 10.2 ± 1.34 |
| HDF-PtNPs | 9.1 ± 2.7 |
| HDF-PdNPS | 8.8 ± 2.1 |
| HDF-AuPtNPs | 10.2 ± 4.1 |
| HDF-AuPdNPs | 18.2 ± 3.4 |

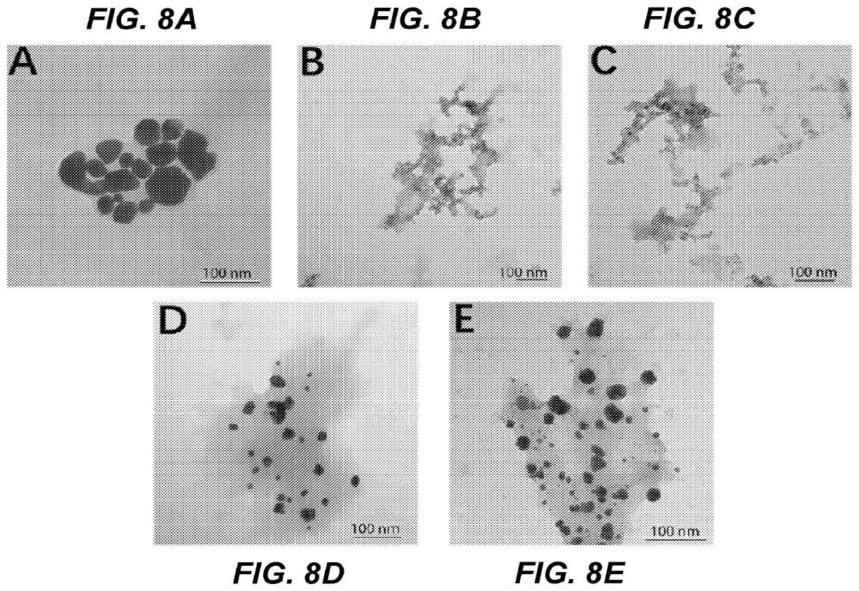

TEM characterization of nanoparticles synthesized by melanoma cells (FIGS. 8A-8E) show nanoparticles coated with organic materials and monodispersed in solution after purification. The sizes of these nanostructures are summarized in Table 2. PtNPs (FIG. 8B) and PdNPs (FIG. 8C) appear as extremely small and amorphous nanostructures embedded in an organic matrix, in contrast with perfectly formed nanostructures of bigger size when they are combined with gold (FIGS. 8D and 8E), as it happened with those made by HDF cells. FIGS. 8A-8E show TEM images of Au(A), Pt(B), Pd(C), AuPt(D), AuPd(E) NPs after purification, biosynthesized from melanoma cells. The size of the MEL-NPs can be in the range from about 1 nm to about 30 nm or 35 nm, from about 15 nm to about 35 nm, from about 5 nm to about 25 nm, from about 10 nm to about 25 nm, from about 10 nm to about 20 nm, and from about 12 nm to about 22 nm. The size can be an average size, determined by average equivalent volume methods (in which the average is volume weighted) or by numerical methods, in which the average is numerically weighted.

TABLE 2
Size distribution of MEL-Au, -Pd, -Pt, -AuPd and -AuPtNPs

| Nanostructure | Diameter (nm) |
|---|---|
| MEL-AuNPs | 12.2 ± 2.6 |
| MEL-PtNPs | 20.3 ± 4.1 |
| MEL-PdNPS | 15.2 ± 3.3 |
| MEL-AuPtNPs | 20.9 ± 2.2 |
| MEL-AuPdNPs | 22.1 ± 8.1 |

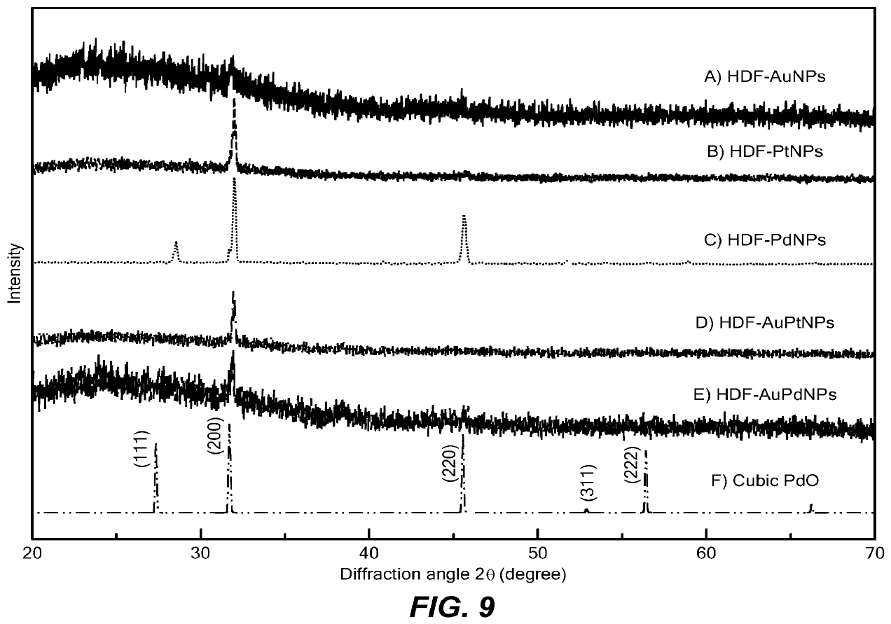

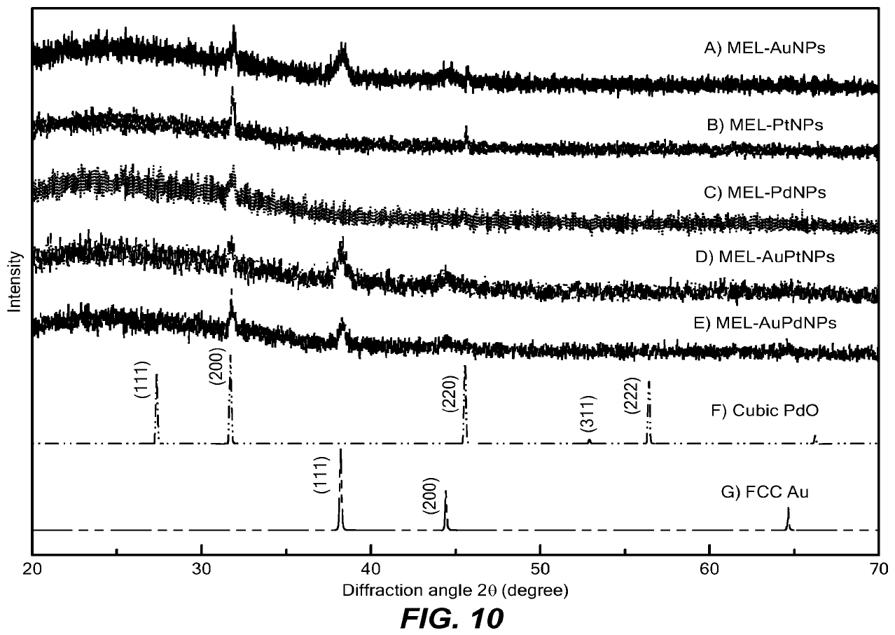

X-ray diffraction (XRD) patterns for the noble metal nanoparticles synthesized using human dermal fibroblasts (HDF) and melanoma (MEL) cells are shown in FIG. 9 and FIG. 10, respectively. The XRD patterns of the noble metal nanoparticles using human dermal fibroblasts (HDF) cells are depicted in FIG. 9. In the case of the Pt- and Pd-based mono- and bimetallic nanoparticles, the experimental diffraction patterns may be principally indexed to their corresponding metal oxides, i.e. cubic PtO and PdO with NaCl-type structures. The sample HDF-AuNPs is amorphous as shown in the top trace of FIG. 9.

Concerning the nanoparticles produced using melanoma (MEL) cells (FIG. 10), the XRD patterns of Au-based mono- and bimetallic nanoparticles presented the characteristic peaks of face-centered cubic (FCC) Au. Furthermore, all the experimental diffraction patterns showed a diffraction peak at around 31.7° (2θ) that may be indexed to the crystallographic plane (200) of the corresponding metal oxides, i.e. cubic PtO and PdO with NaCl-type structures.

To study the stability of the HDF and melanoma cells-synthesized nanoparticles, Z-potential (zeta-potential) measurements of freshly synthesized and 60 days old NPs are shown. As shown in Tables 3 and 4, the nanoparticles can be considered as highly stable because the value of Z-potential doesn't change more than 30 mV. The nanoparticles are unlikely to form aggregates, for example, because of their electrostatic stability.

TABLE 3
Z-potential values for freshly and 60-days
old HDF-Au, -Pd, -Pt, -AuPd and -AuPtNPs

Z-potential (mV)

| Nanostructures | As-synthesized | 60 days old |
|---|---|---|
| HDF-AuNPs | −40.11 ± 2.21 | −36.32 ± 4.41 |
| HDF-PdNPs | −41.23 ± 2.99 | −32.3 ± 3.55 |
| HDF-PtNPs | −38.82 ± 1.9 | −37.09 ± 3.43 |
| HDF-AuPdNPs | −40.17 ± 3.35 | −37.23 ± 3.21 |
| HDF-AuPtNPs | −37.72 ± 2.56 | −35.29 ± 2.36 |

TABLE 4
Z-potential values for freshly and 60-days
old Mel-Au, -Pd, -Pt, -AuPd and -AuPtNPs

Z-potential (mV)

| Nanostructures | As-synthesized | 60 days old |
|---|---|---|
| MEL-AuNPs | −46.87 ± 3.16 | −43.15 ± 4.22 |
| MEL-PdNPs | −45.56 ± 1.32 | −40.11 ± 6.2 |
| MEL-PtNPs | −40.12 ± 3.66 | −31.27 ± 5.11 |
| MEL-AuPdNPs | −42.31 ± 1.97 | −40.99 ± 2.46 |
| MEL-AuPtNPs | −45.98 ± 2.34 | −40.11 ± 5.06 |

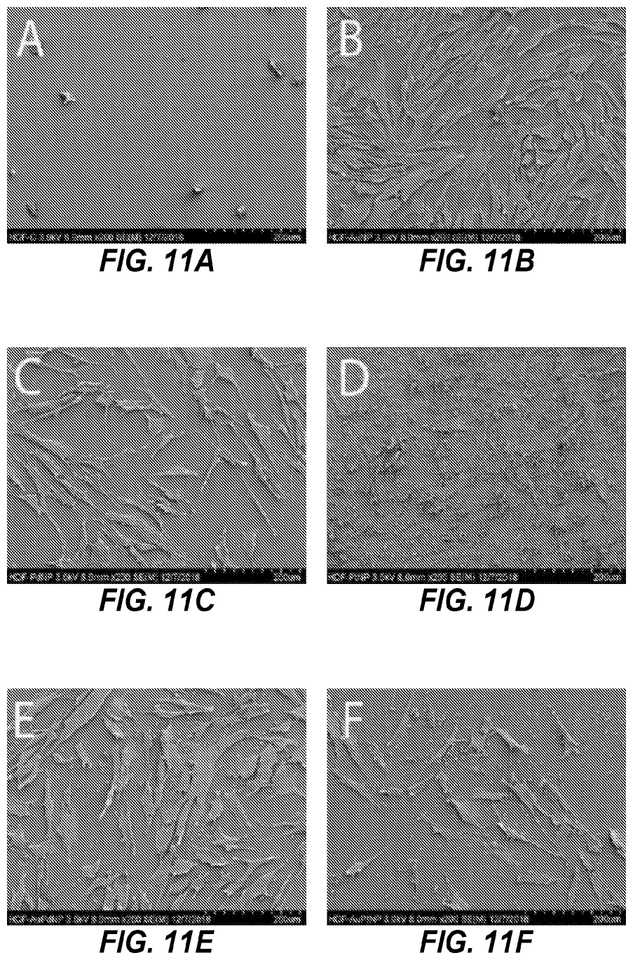

During various studies, cell fixation combined with SEM microscopy technology was used to carry out the synthesis process. After 24 hours incubation with metal solutions with DPBS, the cells were fixed. A control of cells cultured in DPBS (without metals) for 24 hours was also employed. FIGS. 11A-11F shows untreated HDF cells with DPBS (A), and cells cultured with Au (B), Pd (C), Pt (D), AuPd (E) and AuPt (F) metallic salts. As shown in the figures, when observed under low magnification (˜200×), only a few dead cells with a completely destroyed membrane can be found in the control (FIG. 11A), in contrast with perfectly-shaped cells for those experiments in which metallic salts were added and nanoparticle formation allowed (see FIGS. 11B-11F).

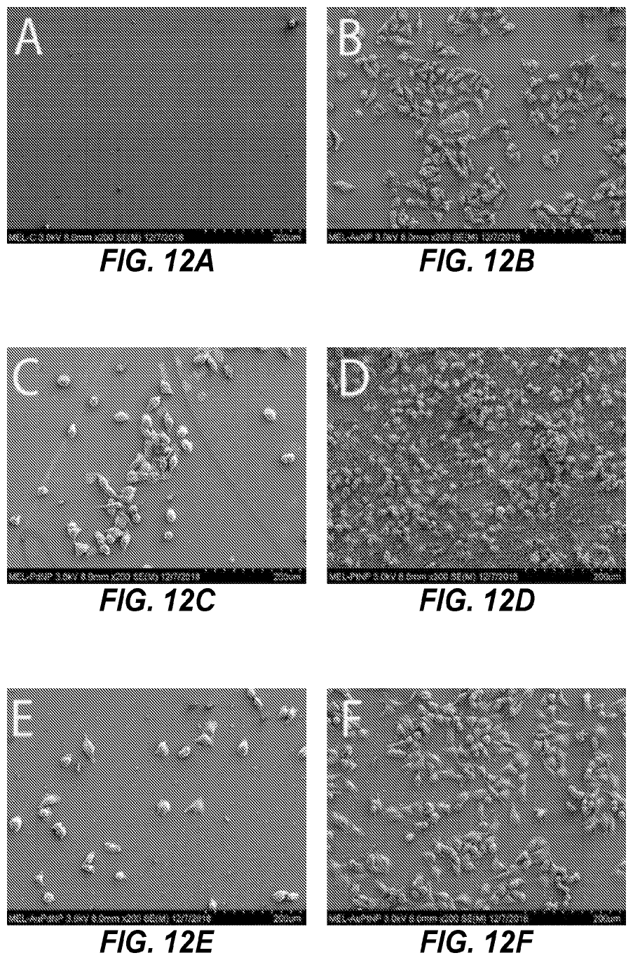

As it can be seen in FIG. 11D, the presence of the Pt metallic salt leads to a high production of extracellular metallic nanoparticles-containing clusters in the extracellular media, what is in accordance with the data obtained in the light microscopy experiments. Similar results were observed for melanoma cell experiments, with an empty control (FIG. 12A) and perfectly shaped cells when they are cultured with Au (FIG. 12B), Pd (FIG. 12C), Pt (FIG. 12D), AuPd (FIG. 12E) and AuPt (FIG. 12F) metallic salts. Once again, a high presence of nanoparticles-organic clusters is found in the samples cultured with Pt metallic salts (FIG. 12D).

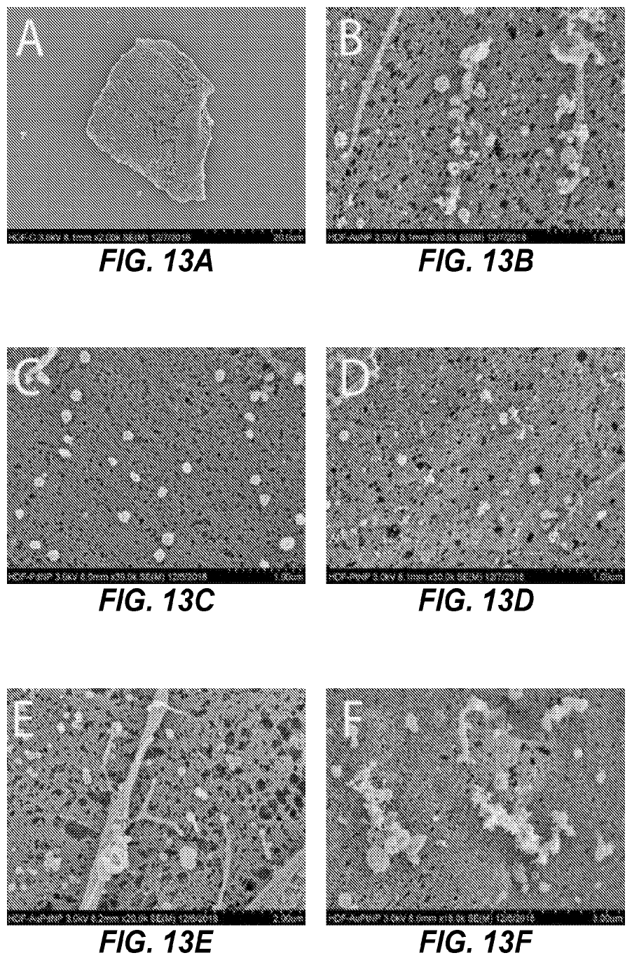

FIGS. 13A-13F show SEM images of HDF cells after 24 hours incubation with only DPBS(A, 2000× magnification), HAuCl₄(B, 30 kX magnification), K₂PdCl₄(C, 30 kX mag.), K₂PtCl₄(D, 30 kX mag.), HAuCl₄ and K₂PdCl₄(E, 20 kX mag.), HAuCl₄ and K₂PtCl₄(F, 18 kX mag.) in DPBS. SEM is a surface analysis method, and the nanoparticles are observed mostly on top of the cell membrane in all the cases (see FIGS. 13B-13F). It is hypothesized that the cell membrane may be a major place where metal ions are reduced to nanoparticles, which is in accordance with the previous hypothesis deduced after analysis of UV-Vis data.

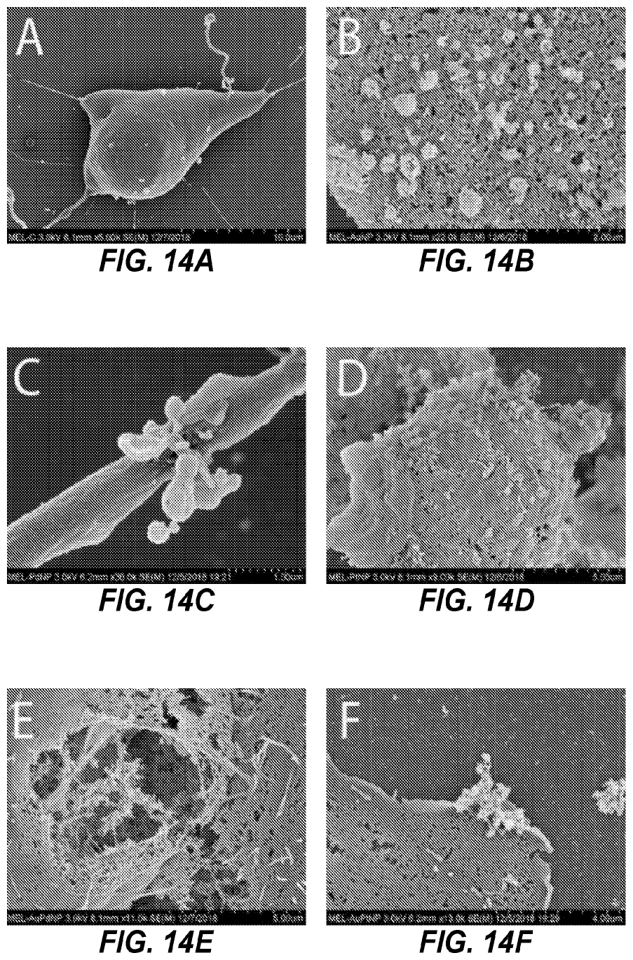

Similar results were obtained when a higher magnification study was applied to the melanoma cells, as can be seen in FIGS. 14A-14F, which show SEM images of melanoma cells after 24 hours incubation with only DPBS(A, 5000× magnification), HAuCl₄(B, 22 kX mag.), K₂PdCl₄(C, 30 kX mag.), K₂PtCl₄(D, 9 kX mag.), HAuCl₄ and K₂PdCl₄(E, 11 kX mag.), HAuCl₄ and K₂PtCl₄(F, 13 kX mag.) in DPBS.

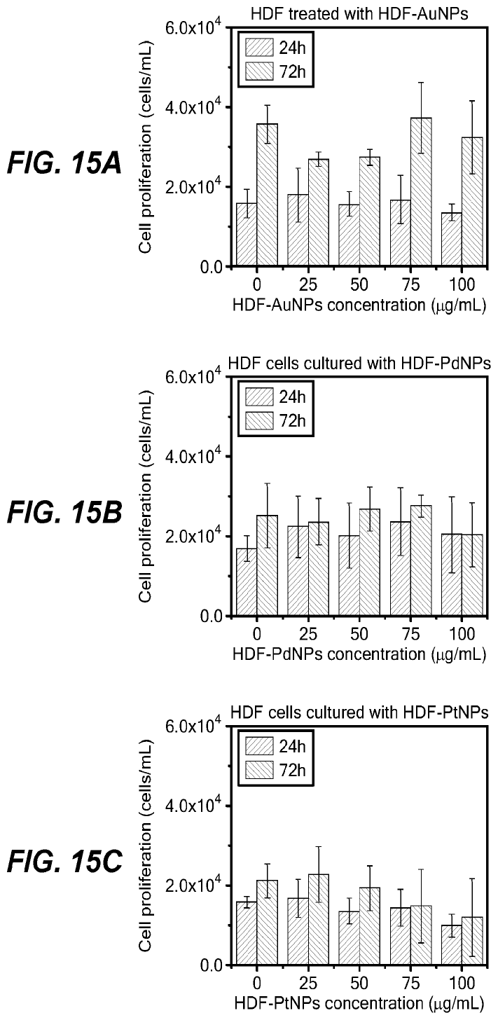

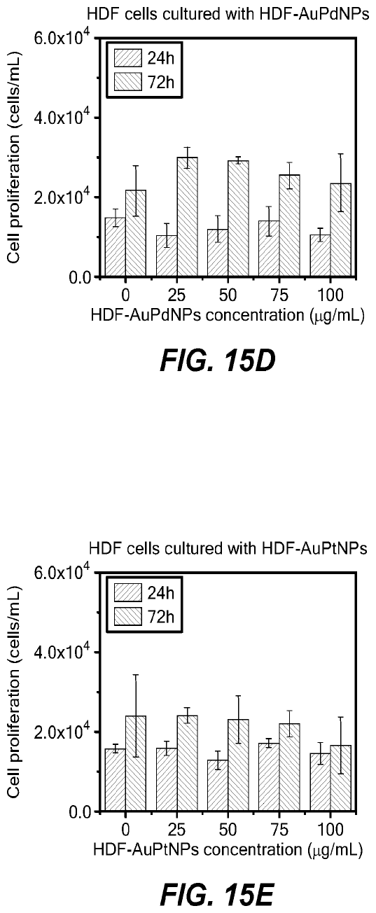

To study the cytotoxicity of HDF- and melanoma cells-synthesized nanoparticles, the nanostructures were added to media and cultured with both HDF and melanoma cells. The in vitro cytotoxicity assays were performed for 24 hours and 72 hours. FIGS. 15A-15E show HDF-AuNPs (A), HDF-PdNPs (B), HDF-PtNPs (C), HDF-AuPdNPs (D), and HDF-AuPtNPs (E) being cultured with HDF cells for 24 hours (left bar plot) and 72 hours (right bar plot). By comparison with the control shown as 0 μg/mL, nanoparticles concentrations between 0 and 100 μg/mL show no significant cytotoxicity towards HDF cells.

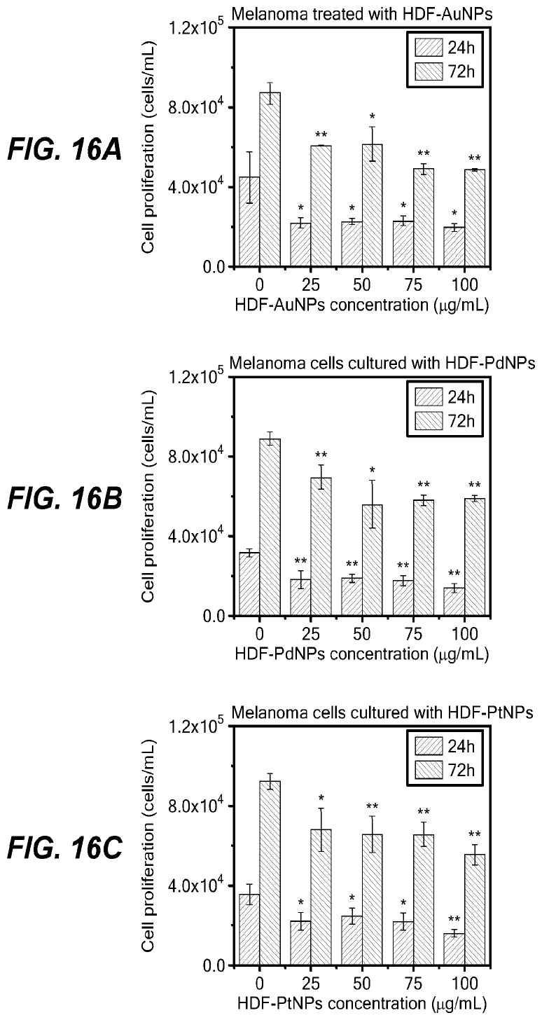

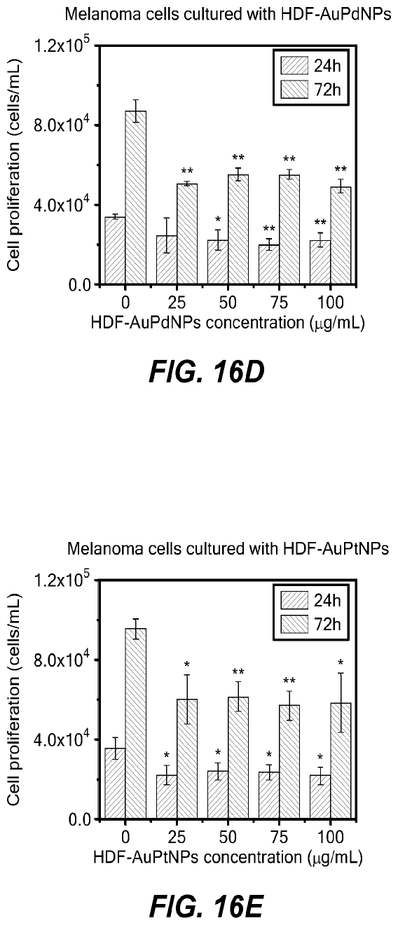

FIGS. 16A-16E show HDF-AuNPs (A), HDF-PdNPs (B), HDF-PtNPs (C), HDF-AuPdNPs (D) and HDF-AuPtNPs (E) being cultured with melanoma cells for 24 and 72 hours. A dose-dependent decay is found within the melanoma cells population, especially noticeable for HDF-PtNPs.

The MIC towards cancer cells shows anticancer activity. The HDF-AuNPs, HDF-PdNPs, HDF-PtNPs, and HDF-AuPtNPs, show a low cytotoxic effect when cultured with HDF cells in a range of concentrations between 25 to 100 μg/mL up to 72 hours. A clear anticancer activity can be found towards melanoma cells within the same concentration ranges. For HDF-AuPdNPs, the anticancer effect was in a concentration range between 50 to 100 μg/mL for a 24 hour treatment, while for 72 hour treatment the concentration range was wider (from 25 to 100 μg/mL) with low cytotoxicity towards HDF cells. Thus, HDF cell synthesized nanoparticles can be considered as valuable anticancer agents in vitro at the concentration of 25 μg/mL for Au-, Pd-, Pt- and AuPtNPs, and 50 μg/mL for AuPdNPs for a 72 hour treatment. The MIC (24 hours) towards cancer cells can be in the range from about 5 to 75 μg/mL, from about 5 to 50 μg/mL, from about 25 to 50 μg/mL, from about 25 to 40 μg/mL, and from about 25 to 35 μg/mL.

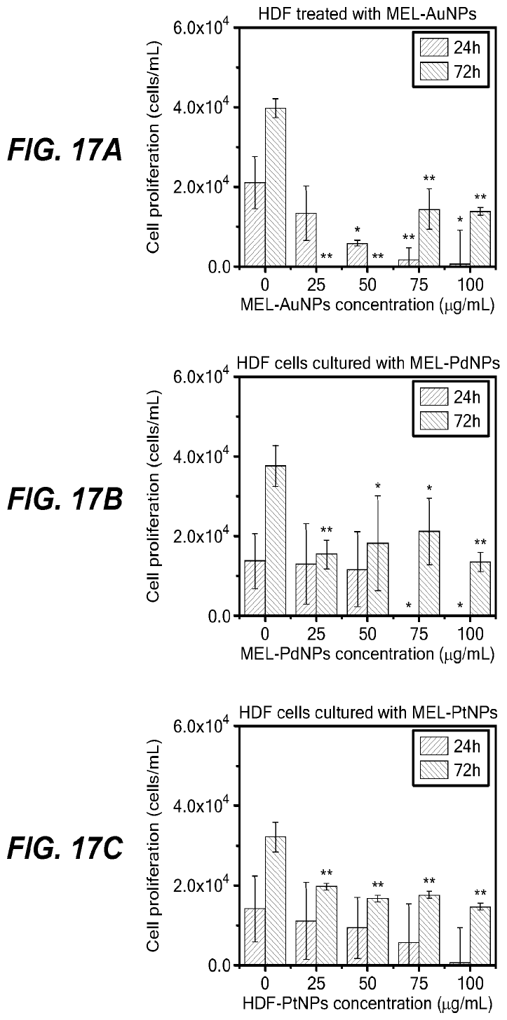

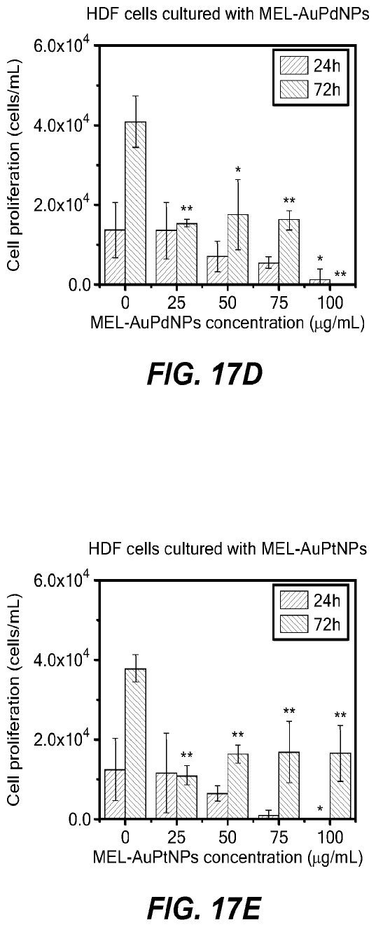

As shown in FIGS. 17A-17E, all melanoma cell synthesized-nanoparticles, MEL-Au (A), MEL-Pd (B), MEL-Pt (C), MEL-AuPd (D), and MEL-AuPtNPs (E), showed a high cytotoxicity towards HDF cells at the concentration between 25 μg/mL to 100 μg/mL for a 72 hour treatment.

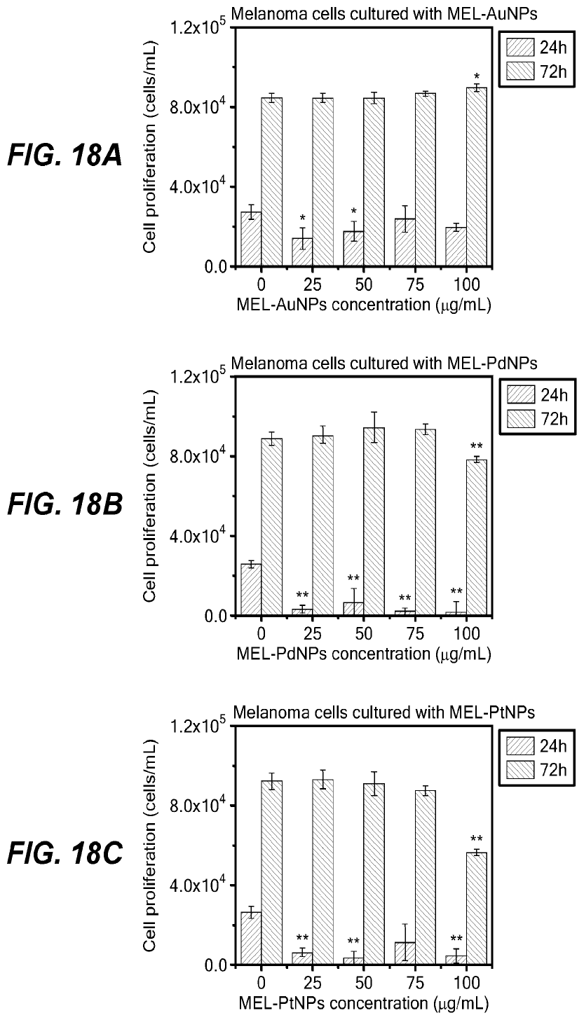

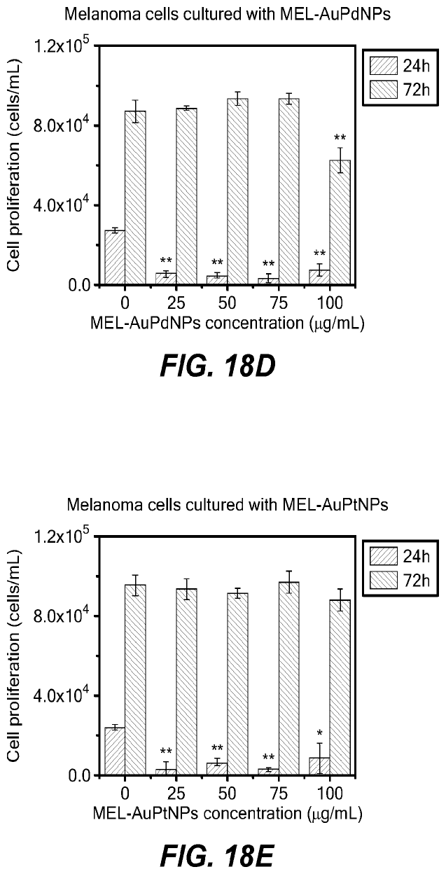

As shown in FIGS. 18A-18E, no anticancer activity is found for a 72 hour treatment, with a cell proliferation that shows no difference between experiment and control, which indicates that these MEL synthesized nanoparticles have no anticancer effect but have high anti-HDF cells activities.

Overall, the results show for the first time the anticancer activity and biocompatibility of human cell mediated nanoparticles. The nanoparticles biosynthesized by HDF cells show anticancer effects towards melanoma cells with low cytotoxicity towards HDF cells. The nanoparticles mediated by melanoma cells show no anticancer activities toward melanoma cells but show high cytotoxicity against HDF cells. It is hypothesized that the anticancer and biocompatible functions of the nanoparticles were associated with the organic coatings on the nanoparticles. The coating from HDF cells can prevent nanoparticle damage HDF cells and can damage melanoma cells. The coating from melanoma cells had converse properties. The reason behind these properties remains unknown at this time, but more experiments can be conducted in the future to elucidate this behavior. Besides, based on the results obtained, the HDF-mediated metallic nanoparticles have an important value as biomedical agents, a reason why further experiments were triaged. The MEL-mediated nanoparticles may still prove useful as targeting agents towards cancer cells or as imaging agents towards cancer cells.

In order to further study the cytotoxicity of the HDF mediated nanoparticles, IC₅₀ values are calculated and plotted in Table 5. The IC₅₀ (24 hours) towards cancer cells can be in the range from about 10 to 100 μg/mL, from about 20 to 75 μg/mL, from about 25 to 70 μg/mL, from about 35 to 60 μg/mL, and from about 35 to 55 μg/mL.

TABLE 5
IC₅₀ values for different HDF synthesized
nanoparticles cultured with melanoma cells.

| IC₅₀ (μg/mL) | 1 day | 3 days |
|---|---|---|
| HDF-AuNPs | 33.35 | — |
| HDF-PdNPs | 55.96 | 1.504 |
| HDF-PtNPs | — | — |
| HDF-AuPdNPs | 58.78 | 1.439 |
| HDF-AuPtNPs | 61.35 | — |

(The data represented by “—” does not fit with the regression).

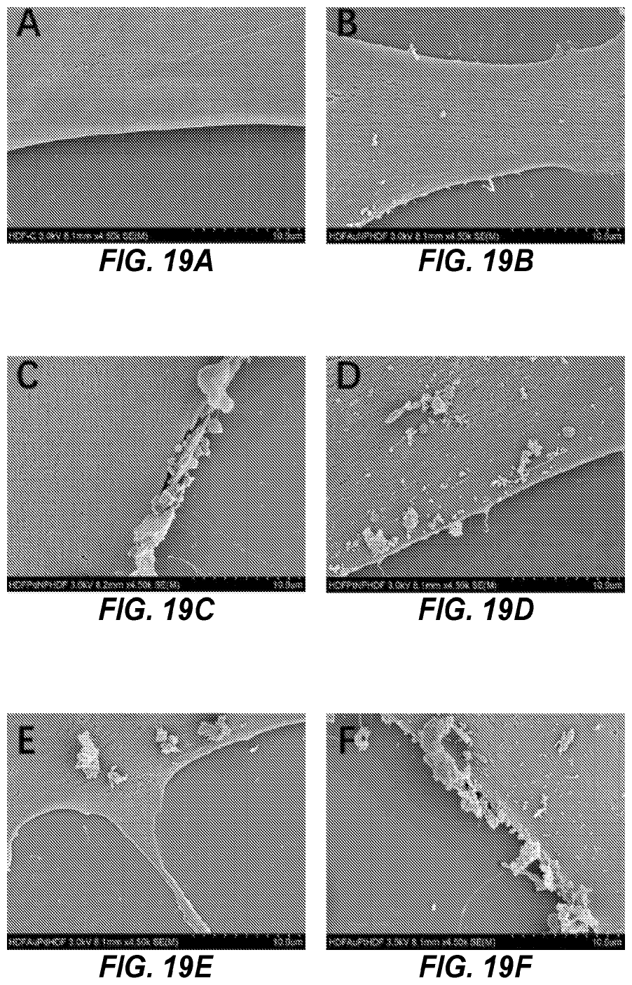

Cell fixation studies of the cytotoxic effect of HDF-mediated nanoparticles were designed. The effect of 24 hours incubation with only EMEM (A), HDF-AuNPs (B), HDF-PdNPs (C), HDF-PtNPs (D), HDF-AuPdNPs (E), and HDF-AuPtNPs (F) was evaluated using cell fixation and SEM images, as can be seen in FIGS. 19A-19F for HDF cells. As is shown in FIGS. 19A-19F, when HDF cells were treated with HDF synthesized nanoparticles, no significant modification on the cell morphology can be found compared to the control group shown in FIG. 19A. Therefore, the results suggest the nanoparticles do not have considerable affluence on cell proliferation, in accordance with the low cytotoxicity observed after analysis of MTS assays.

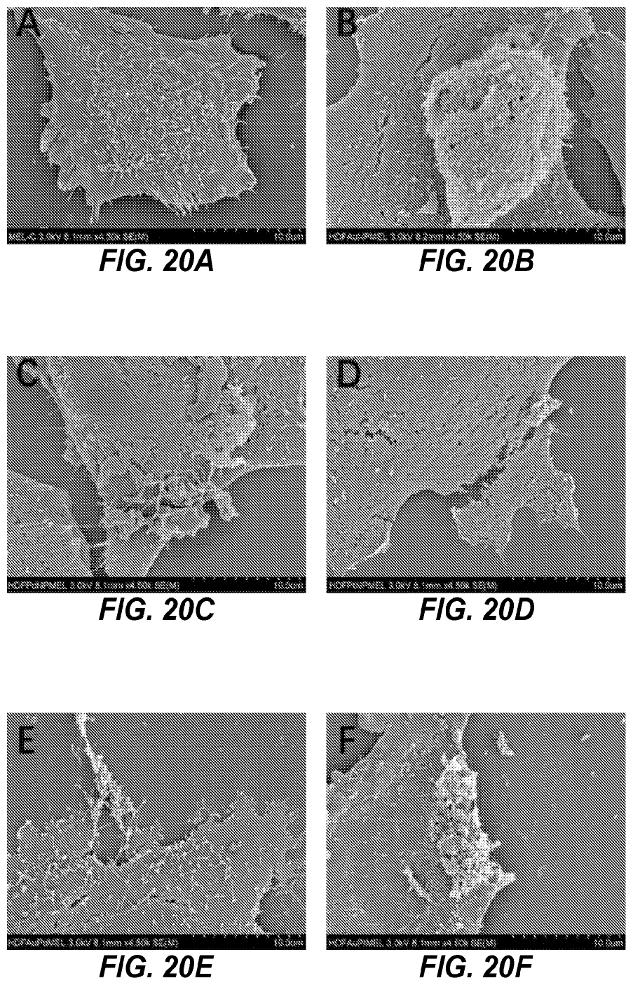

On the contrary, it is shown in FIGS. 20A-20F, when melanoma cells are treated with the same HDF-synthesized nanoparticles, a significant change of morphology was observed when compared to the control group (FIG. 20A). Discontinuous areas on the membrane can be seen for melanoma cells treated with HDF-AuNPs (B), HDF-PdNPs (C), HDF-PtNPs (D), HDF-AuPtNPs (E), and HDF-AuPdNPs (F), compared to the control (DMEM, FIG. 20A). The findings suggest that cell death might be related to a necrosis mechanism. HDF-AuNPs are able to cause swelling of the membrane of melanoma cells, due to the rearranging of the structures in the cytoskeleton (FIG. 20B). The cell membranes were eventually disrupted, leading to cell death.

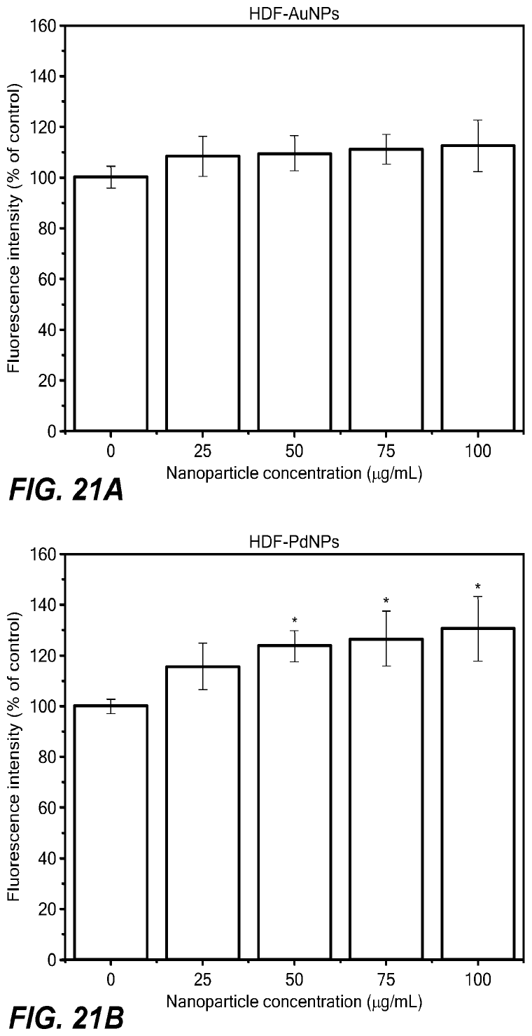

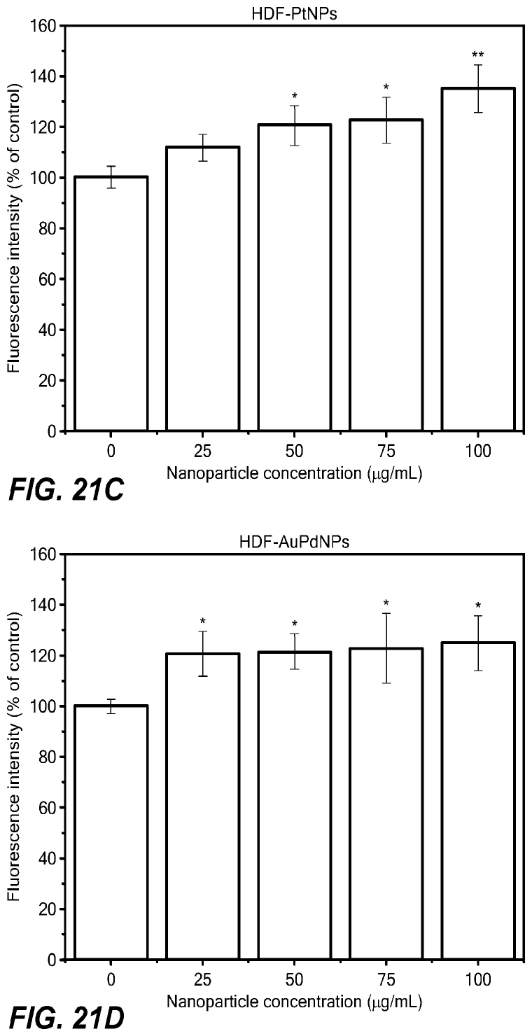

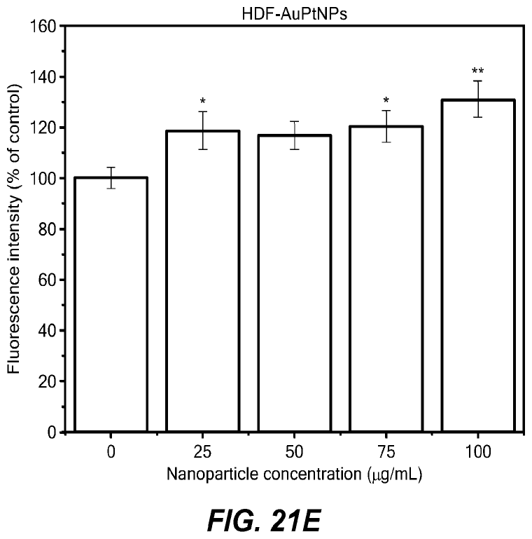

To further investigate the activity, reactive oxygen species (ROS) analyses were designed. ROS analysis (FIGS. 21A-21E) shows a dose-dependent increase in ROS production when the HDF-PdNPs (B), HDF-PtNPs (C), HDF-AuPdNPs (D), and HDF-AuPtNPs (E) are presented within the cellular media. On the other hand, the presence of HDF-AuNPs (FIG. 21A) did not trigger a significant increase in ROS production, which indicate that the anticancer effect of these particular nanoparticles might be related to another unidentified mechanism.

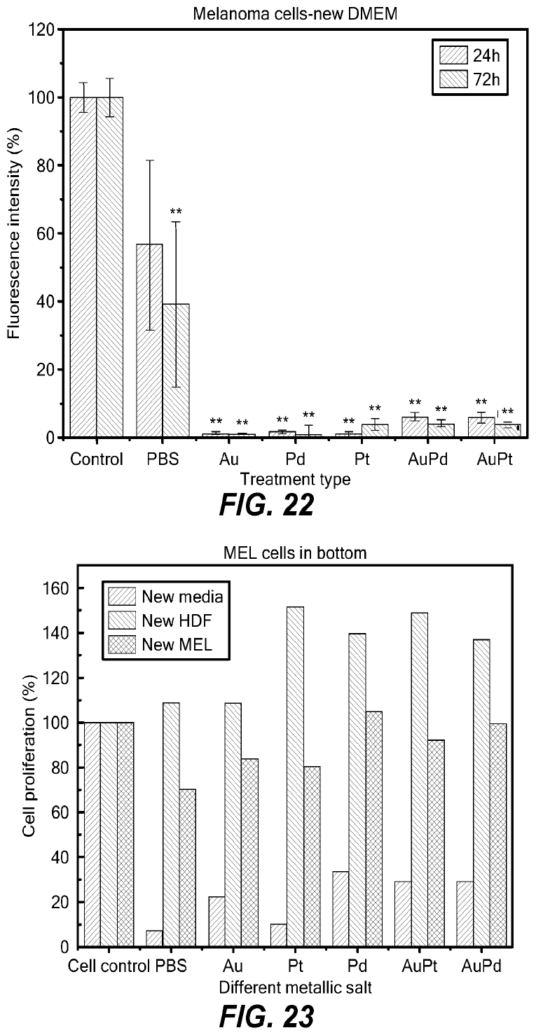

A cell proliferation study was conducted with the objective of the study to show that melanoma cells do not proliferate after they are treated with a metallic salt concentration and produced metallic nanoparticles. Using MTS assays, FIG. 22 shows the differences in terms of cell proliferation between control of melanoma cells growing in presence of DMEM (named as control on the X-axis) and melanoma cells cultured in PBS for 24 hours and placed back in DMEM media (named as PBS on the X-axis). Melanoma cells that were able to produce nanoparticles did not show a change in the proliferation compared to these positive and negative controls when they were placed in regular DMEM media after production of metallic nanoparticles. Therefore, it is possible to state that the cells that produce metallic nanoparticle are not able to proliferate anymore.

To further investigate, a new media, new HDF cells, and new melanoma cells experiment was designed. The method is: seed melanoma or HDF cells in DMEM in 96 well plates, with cell density of 5×10⁴ cells/well. Put them in incubator under standard condition (37° C. with 5% CO₂) and let them grow for 24 hours. Then remove the media, add DPBS to wash once, then add metallic salt (Au, Pt, Pd, AuPt, or AuPd) with a positive control in which is added media and a negative control in which is added DPBS. Then put them in incubator under standard conditions for 24 hours. After that, remove the supernatant, then divide the cells into 3 groups. Group 1: Add new DMEM; Group 2: Add new HDF cells on top of these cells that were able to produce nanoparticles, with the new HDF cells density of 5×10⁴ cells/well; Group 3: Add new melanoma cells on top of these cells that were able to produce nanoparticles, with new melanoma cells density of 5×10⁴ cells/well. Let them grow under standard condition for 24 hours. Then remove the supernatant, add MTS with DMEM with the ratio of 1-part MTS to 5 parts DMEM. Finally, wait 4 hours then measure absorbance.

As shown in FIG. 23 in the left bars plotted, in new media experiment, after adding metallic salt, the growth of melanoma cells was stopped, as compared to the light microscopy data, the cells maintained their morphology, however it can be hypothesized that they lose the function of growth; thus it is more like a biomaterial (at that point) rather than cells. In FIG. 23, center bars plotted, when new HDF cells were added on top of the cells that were able to produce nanoparticles, HDF cells eventually grow. The HDF cells will grow faster when they are added on top of the melanoma cells that were able to produce PtNPs. Moreover, new melanoma cells on top grow slower than HDF cells, as can be seen by the right bars plotted in FIG. 23.

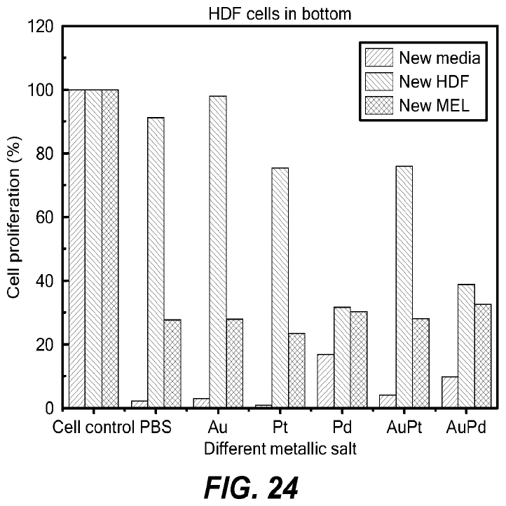

As shown in FIG. 24 in the left bars plotted, in the new media experiment, after adding new DMEM, the HDF cells that were able to produce nanoparticles stop growing which shows a similar result with FIG. 23. More, when new HDF cells were added on top (center bar plotted in FIG. 24), new HDF cells grow and they grow faster on top of the HDF cells that were able to produce AuNPs. However, when new melanoma cells were added on top (right bars plotted in FIG. 24), new melanoma cells grow slower than new HDF cells.

From FIGS. 23-24, The metallic salt can stop the growth of both melanoma and HDF cells. Nevertheless, when new HDF cells or new melanoma cells were added on top of them, new HDF cells grow better. It is hypothesized the cells that were able to produce nanoparticles lose growth function and become a bio-composite on which new HDF cells grow better. Also, in accordance to the MTS results, it is suggested that the reason HDF cells grow better is because the biosynthesized nanoparticles will damage melanoma cells in 24 hour results. The nanoparticles synthesized from HDF cells are highly biocompatible with HDF cells.

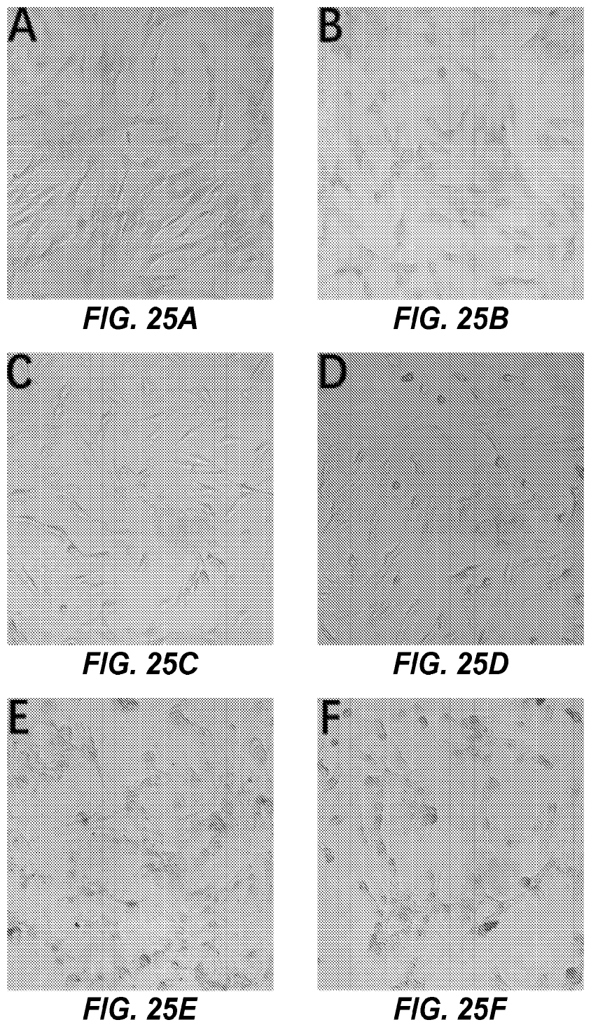

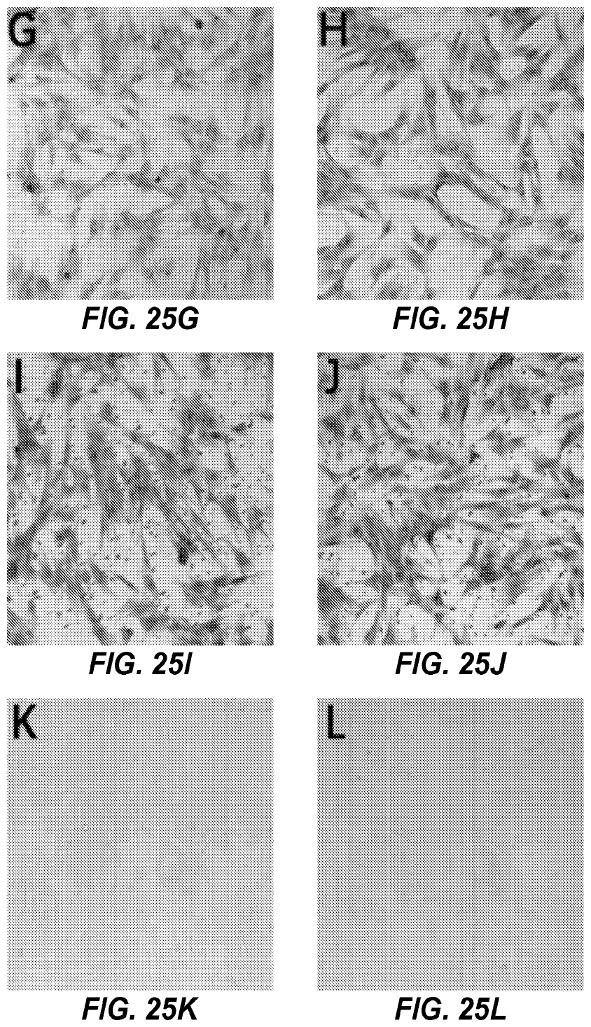

Resistance studies were carried out under extreme conditions. FIGS. 25A-25L show HDF cells at 0 hours (left side, A, C, E, G, I, K) and 24 hours (right side, FIG. 25B, D, F, H, J, L) after the inoculation of the cell media with the metal (and control) conditions with highly acidic conditions. For highly-acidic conditions, the liquid phase was removed from the plates, followed by the addition of a highly-acidified DPBS at a pH 1±0.2. Subsequently, the plates were placed inside an incubator at standard conditions. Light microscopy characterization was conducted over the two sets of experiments at 0 and 24 hours. As it can be seen, the cells treated with different metallic salts: Au (FIG. 25A to 25B), Pt (FIG. 25C to 25D), Pd (FIG. 25E to 25F), AuPt (FIG. 25G to 25H) and AuPd (FIGS. 25I to 25J), and allowed to produce nanoparticles remained attached to the bottom and did not lost their morphology as a consequence of the presence of metallic nanoparticles. On the other hand, those cells that were not treated with any metallic salt (see FIG. 25K to 25L) lost their structure as a consequence of the disruption of the cell membranes and release of the cytoplasmic content due to the extreme environmental conditions.

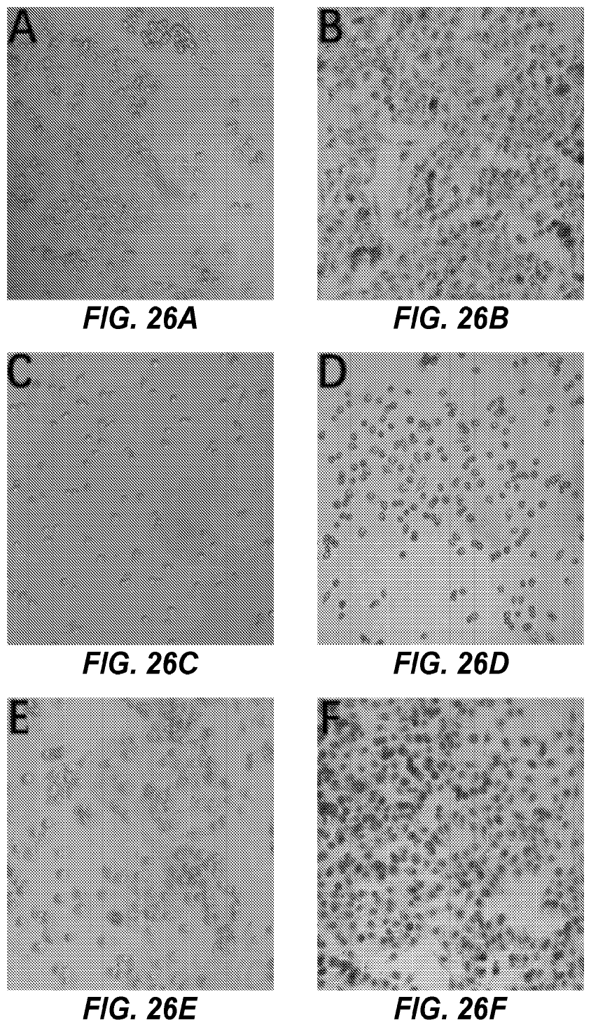

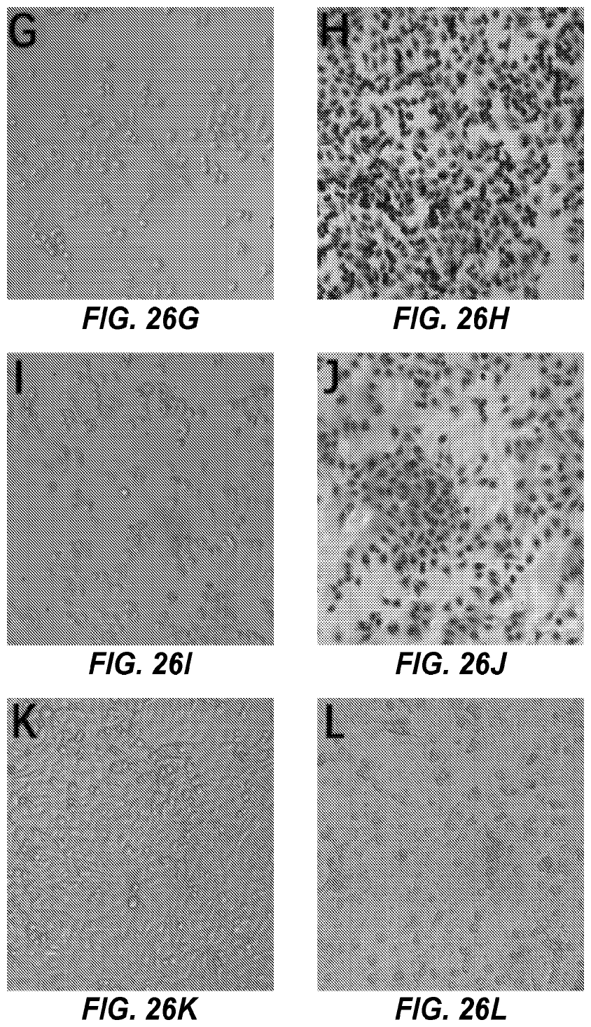

FIGS. 26A-26L show melanoma cells at 0 (left side, A, C, E, G, I, K) and 24 hours (right side, FIG. 26B, D, F, H, J, L) after the inoculation of the cell media with highly acidic conditions. As it can be seen, the cells treated with different metallic salts: Au (FIG. 26A to 26B), Pt (FIG. 26C to 26D), Pd (FIG. 26E to 26F), AuPt (FIG. 26G to 26H) and AuPd (FIGS. 26I to 26J), and allowed to produce nanoparticles remained attached to the bottom and did not lose their morphology as a consequence of the presence of metallic nanoparticles. On the other hand, those cells that were not treated with any metallic salt (K to L) lost their structure as a consequence of the disruption of the cell membranes, going from a perfectly shaped conglomerate of cells to clusters of dead cells. In general, it can be observed that the acidic conditions have a higher impact in the morphology of HDF cells compared to melanoma; and the cells that were allowed to produce nanoparticles did not suffer important changes from the extreme environmental conditions.

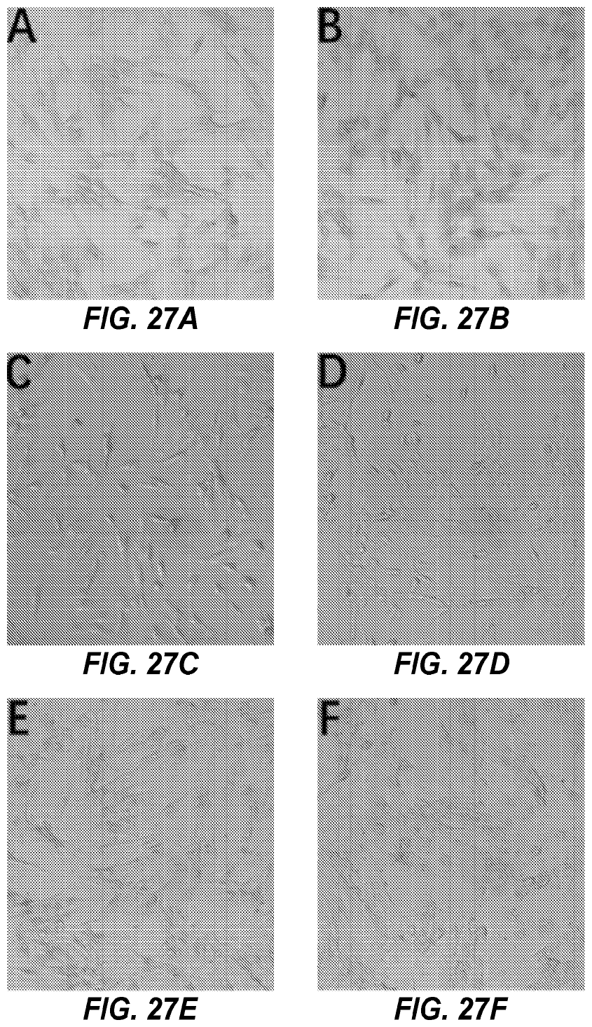

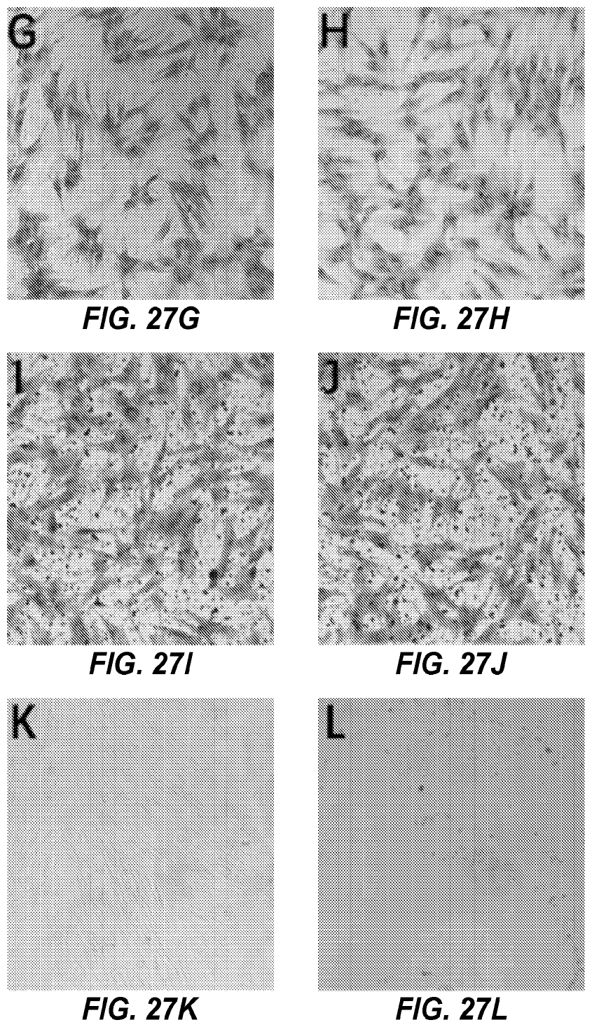

FIGS. 27A-27L show HDF cells at 0 hours (left side, A, C, E, G, I, K) and 24 hours (right side, FIG. 27B, D, F, H, J, L) after the inoculation of the cell media with the metal (and control) conditions and highly basic conditions. For highly-basic conditions, the liquid phase was removed from the plates, followed by the addition of a highly-basified DPBS with a highly basic NaOH environment (pH˜13). Subsequently, the plates were placed inside an incubator at standard conditions. Light microscopy characterization was conducted over the two sets of experiments at 0 and 24 hours. As it can be seen, the cells treated with different metallic salts: Au (FIG. 27A to 27B), Pt (FIG. 27C to 27D), Pd (FIG. 27E to 27F), AuPt (FIG. 27G to 27H) and AuPd (FIGS. 27I to 27J), and allowed to produce nanoparticles remained attached to the bottom and did not lose their morphology as a consequence of the presence of metallic nanoparticles. On the other hand, those cells that were not treated with any metallic salt (see FIG. 27K to 27L) lost their structure as a consequence of the dissolving of the cell membranes and release of the cytoplasmic content due to the extreme environmental conditions.

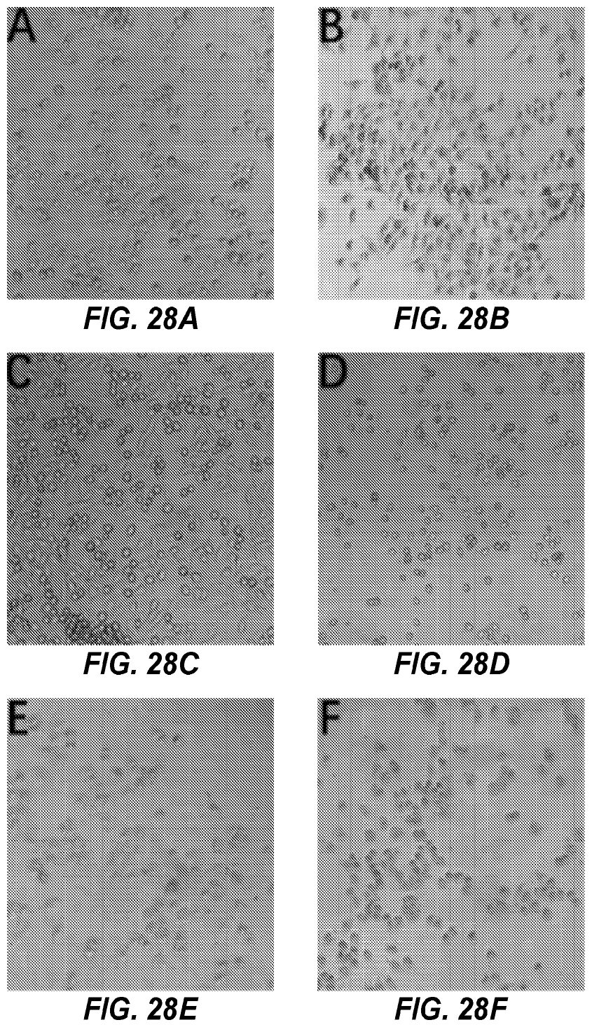

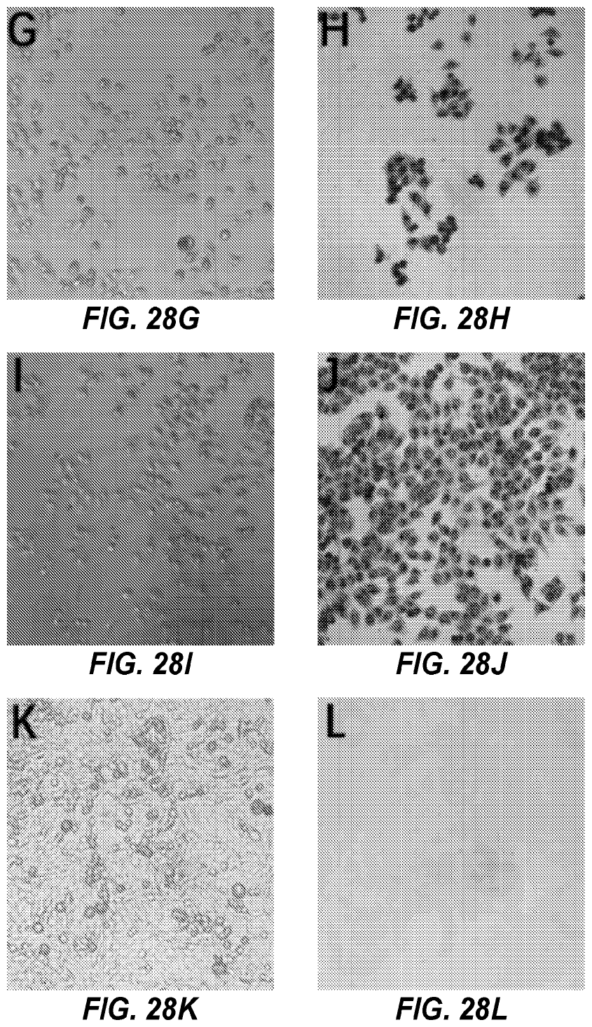

FIGS. 28A-28L show melanoma cells at 0 (left side, A, C, E, G, I, K) and 24 hours (right side, FIG. 28B, D, F, H, J, L) after the inoculation of the cell media with highly basic conditions (NaOH, pH˜13). As it can be seen, the cells treated with different metallic salts: Au (FIG. 28A to 28B), Pt (FIG. 28C to 28D), Pd (FIG. 28E to 28F), AuPt (FIG. 28G to 28H) and AuPd (FIGS. 28I to 28J), and allowed to produce nanoparticles remained attached to the bottom and did not lose their morphology as a consequence of the presence of metallic nanoparticles. On the other hand, those cells that were not treated with any metallic salt (K to L) lost their structure as a consequence of the dissolving of the cell membranes, going from a perfectly shaped conglomerate of cells to clusters of dead cells. Therefore, it can be considered that the basic conditions have high impact in the presence of both HDF and melanoma cells. It was hypothesized that the nanoparticles that the cells produced may protect them from extreme environmental surroundings.

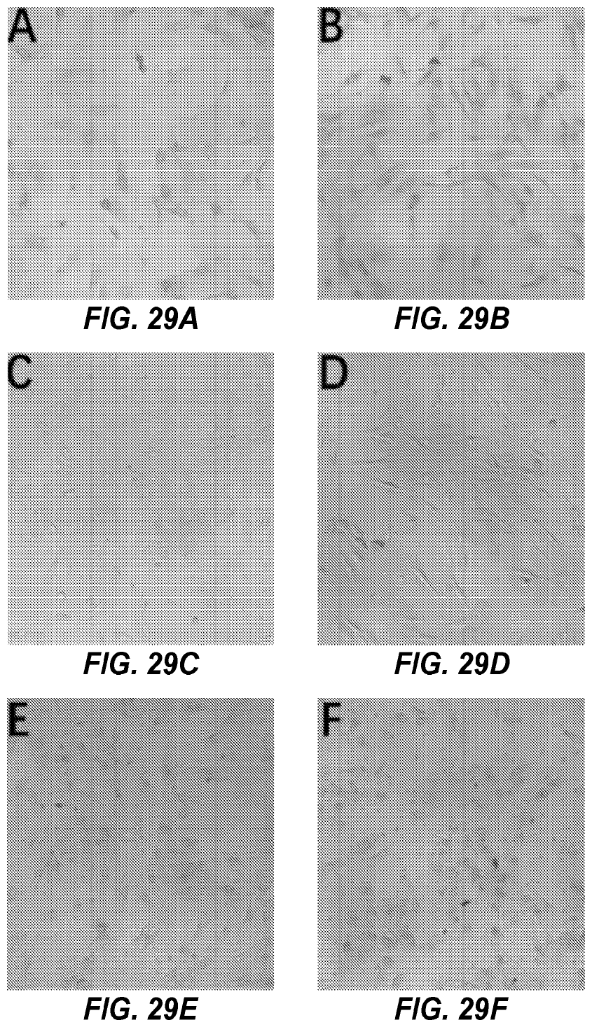

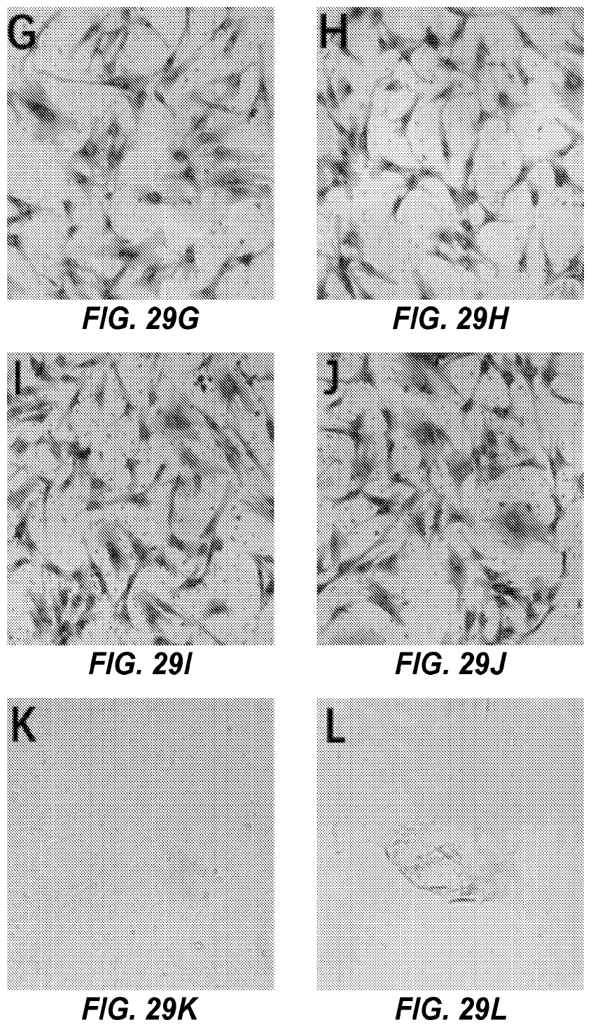

As shown in FIGS. 29A-29L, HDF cells incubated in NaCl salt supersaturation conditions for 0 (left side, A, C, E, G, I, K) and 24 hours (right side, B, D, F, H, J, L), the metallic solution-treated cells, Au (A to B), Pt (C to D), Pd (E to F), AuPt (G to H), and AuPd (I to J), maintained their morphology due to the presence of metallic nanoparticles while the untreated cells (K to L) lost their structural identity due to the high concentration of salt.

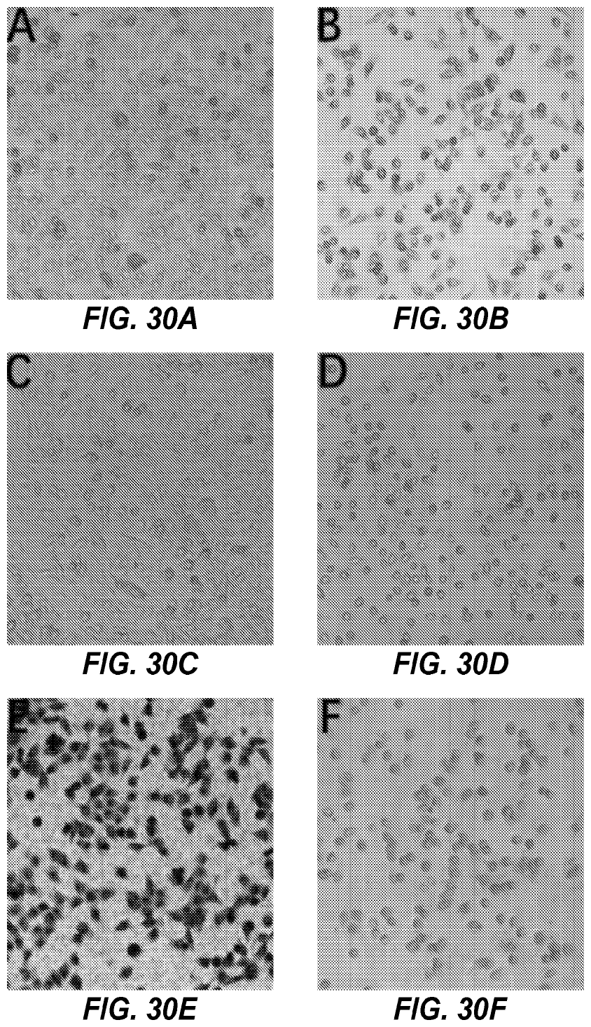

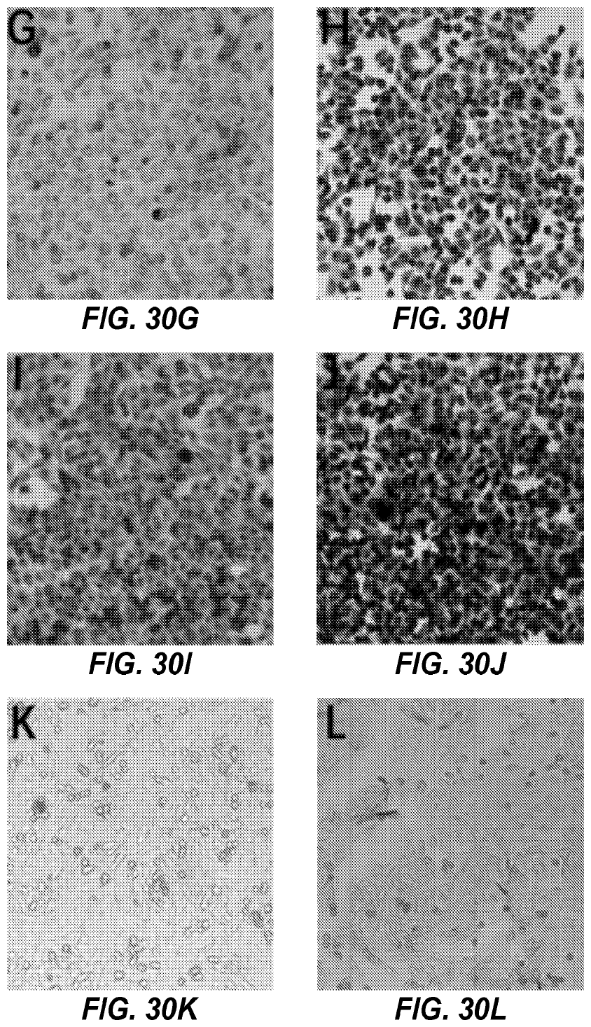

In FIGS. 30A-30L, melanoma cells at 0 (left side, A, C, E, G, I, K) and 24 hours (right side, B, D, F, H, J, L) are shown. In the Pt (C to D) treated solution group, the melanoma cells were detached from the bottom due to the high concentration of salt conditions. However, the cells treated with Au (A to B), Pd (E to F), AuPt (G to H) and AuPd (I to J) kept their morphology. What is more, untreated melanoma cells (K to L) started dying due to the high concentration of salt in the media. It can be found that the high concentration of salt conditions has a higher impact on the morphology of HDF cells than melanoma cells. Besides, it seemed like the melanoma cells treated with Pt solution suffered from extreme conditions while the others were not.

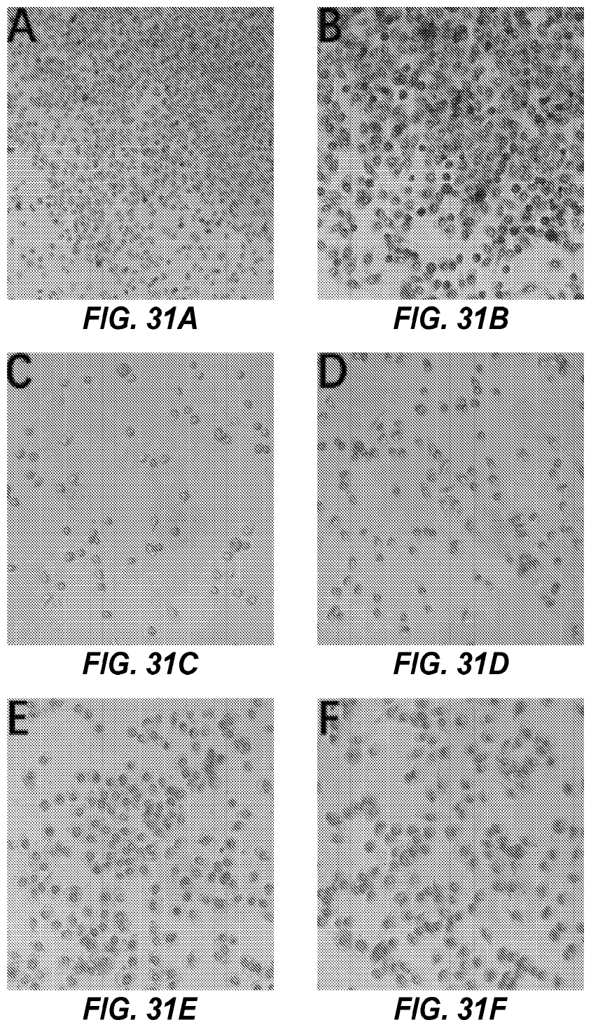

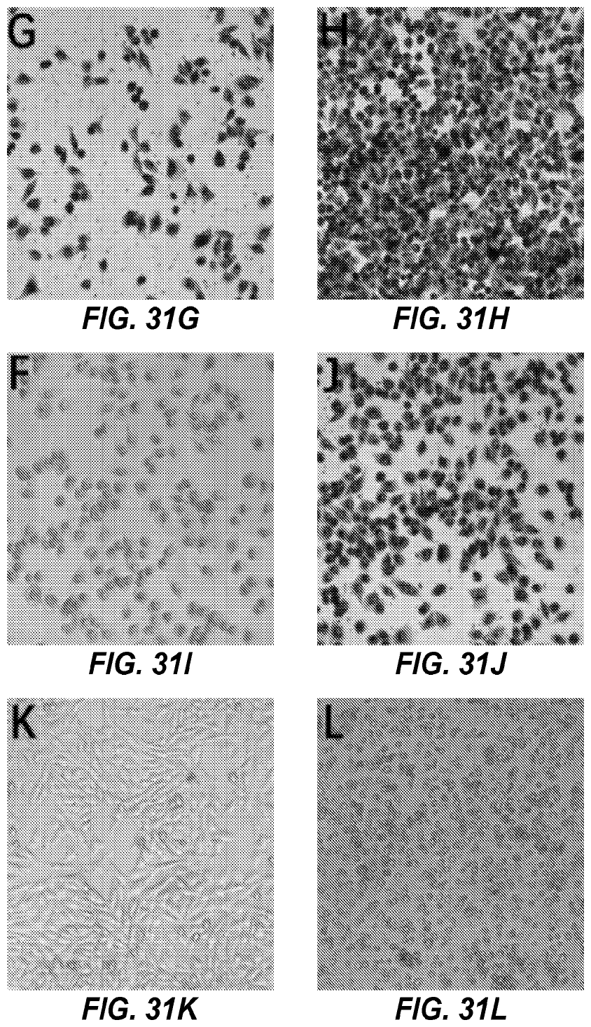

As shown in FIGS. 31A-31L, the HDF cells at 0 (left side) and 24 hours (right side) after incubation with autoclaved DI water are compared. As can be seen, the cells treated with different metallic solutions, Au (A to B), Pt (C to D), Pd (E to F), AuPt (G to H) and AuPd (I to J), kept their morphology due to the presence of nanoparticles. On the contrary, untreated cells (K to L) lost their morphology and detached from the bottom after 24 hours incubation due to the aqueous phase conditions.

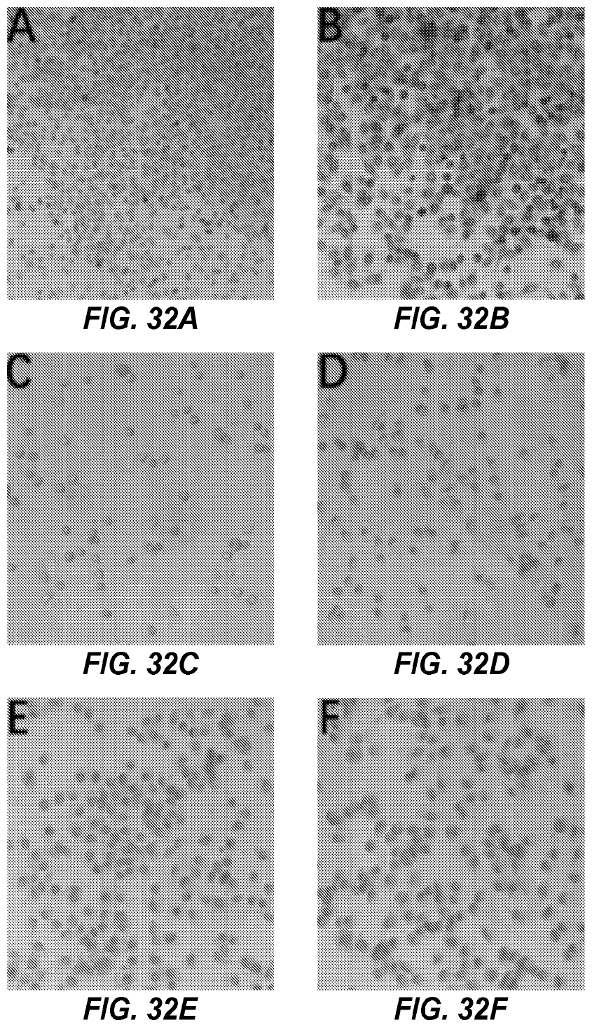

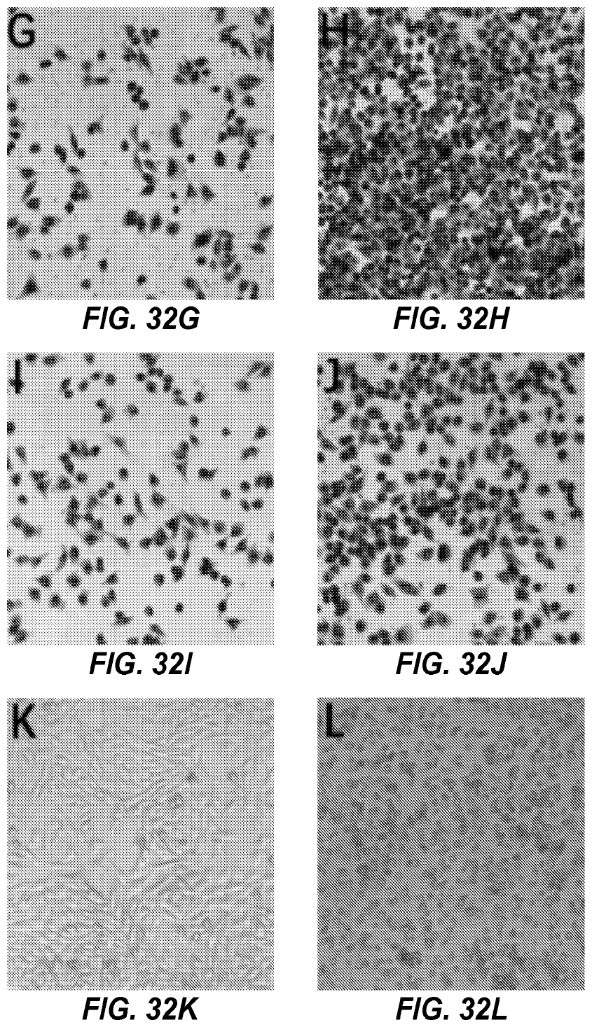

Moreover, the melanoma cells incubated with autoclaved DI-water (FIGS. 32A-32L) after 0 (left side) and 24 hours (right side) are compared. The melanoma cells treated with different metallic solutions, Au (A to B), Pt (C to D), Pd (E to F), AuPt (G to H) and AuPd (I to J), kept their morphology after 24 hours incubation with DI-water as the consequence of the synthesized nanoparticles. On the other hand, the untreated cells (K to L) became spherical and detached from the bottom after 24 hours incubation due to the aqueous phase conditions.

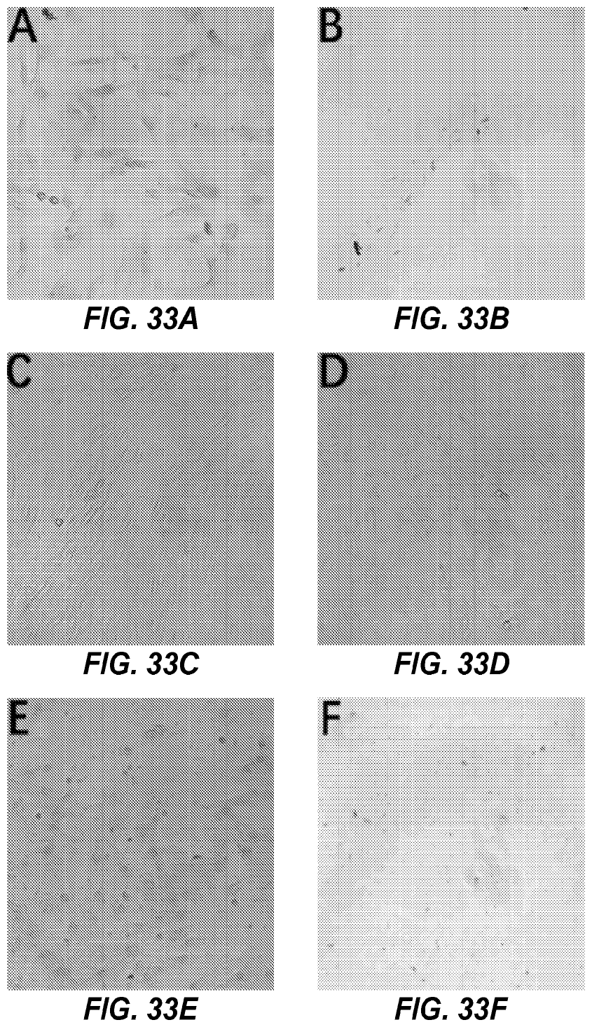

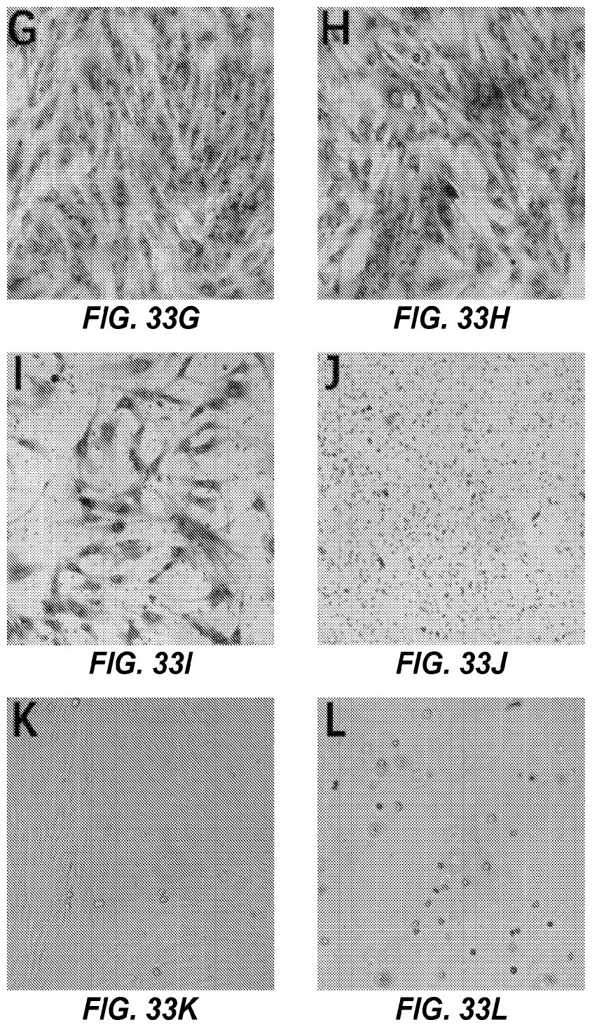

FIGS. 33A-33L show HDF cells at 0 (left side) and 72 hours (right side) after the inoculation with concentrated trypsin conditions. As it can be seen, the cells treated with different metallic salts, Pt (C to D) and AuPt (G to H), and allowed to produced nanoparticles remained attached to the bottom and did not lose their morphology. On the other hand, those cells that were treated with Au (A to B), Pd (E to F) and AuPd (I to J) metallic salts dissolved in the high concentration of trypsin conditions, while the untreated cells (K to L) lose their structure and are detached from the bottom.

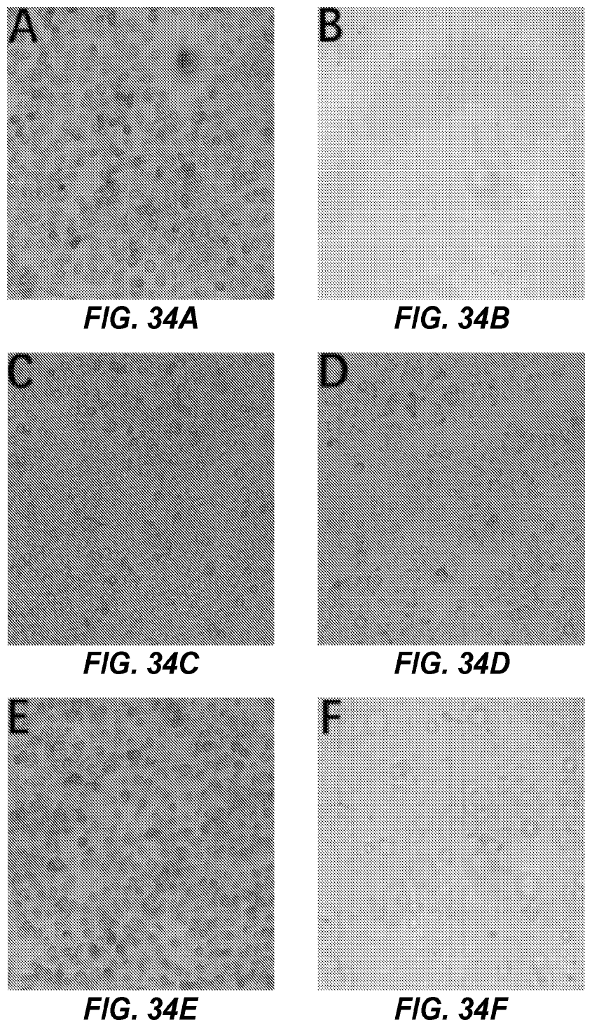

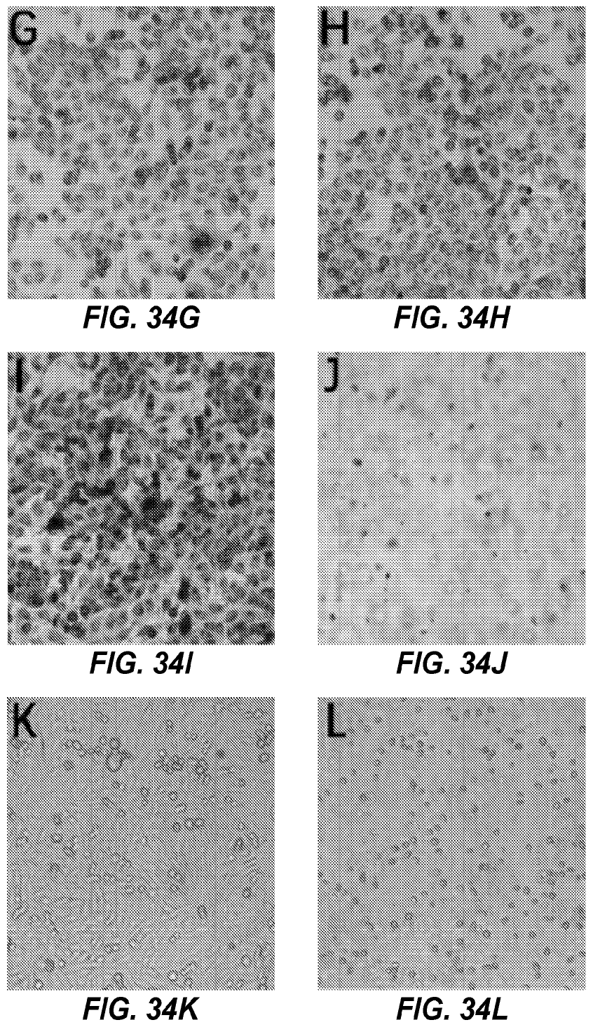

FIGS. 34A-34L shows melanoma cells at 0 (left side) and 72 hours (right side) after the inoculation with concentrated trypsin. As it can be seen, the cells treated with different metallic salts, Pt (C to D) and AuPt (G to H), remained attached to the bottom and did not lose their morphology. On the other hand, those cells treated with Au (A to B), Pd (E to F), AuPd (I to J) dissolved while the untreated cells (K to L) lose their structure and are detached from the bottom to the extreme environmental conditions.

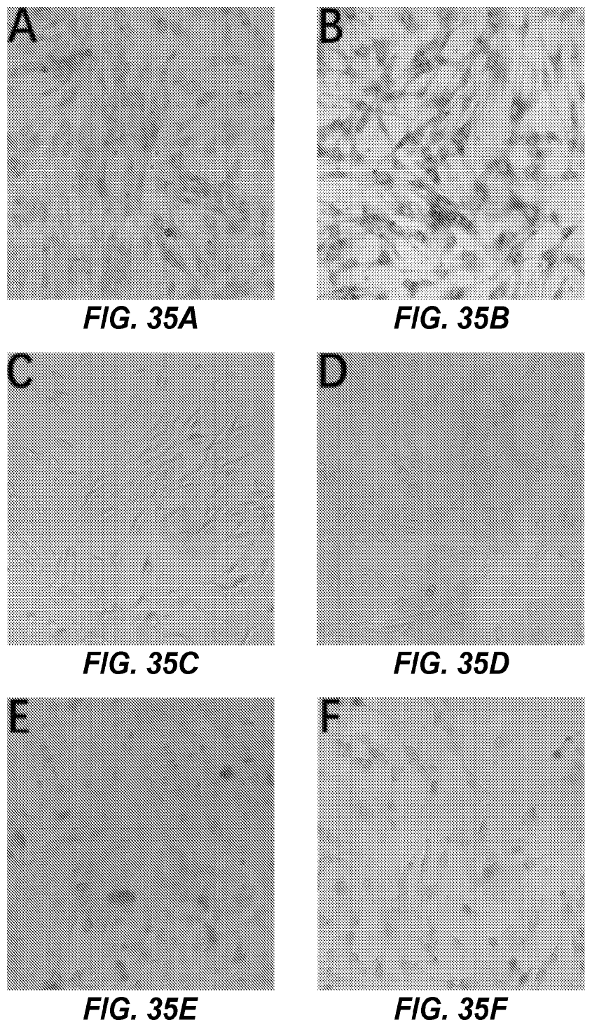

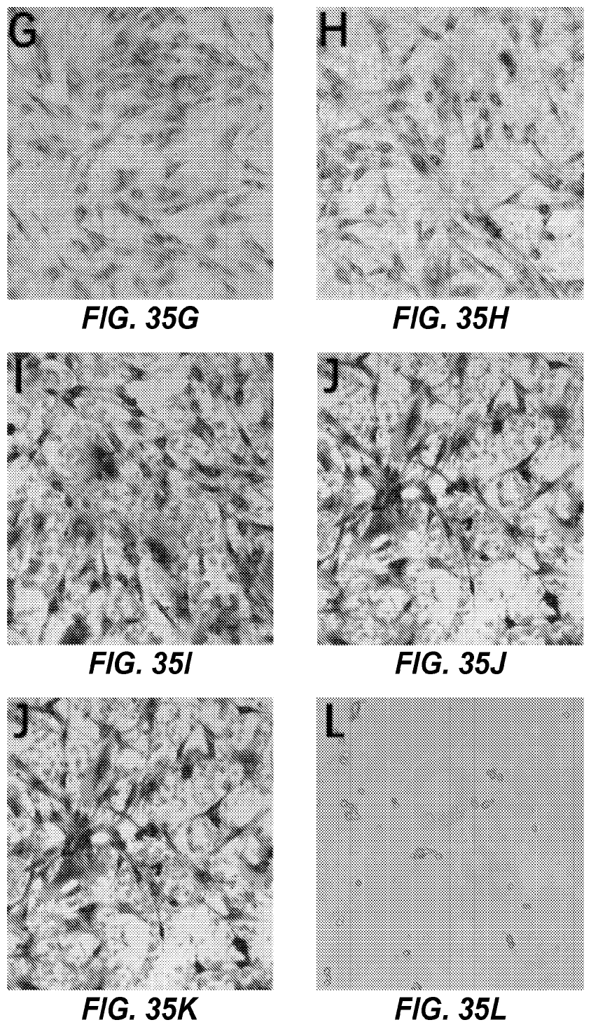

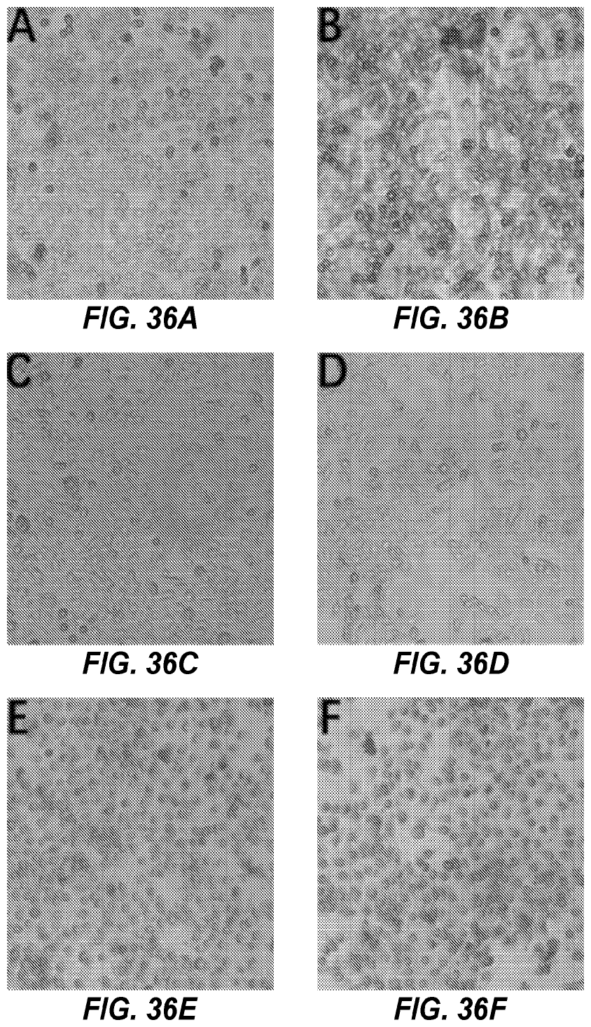

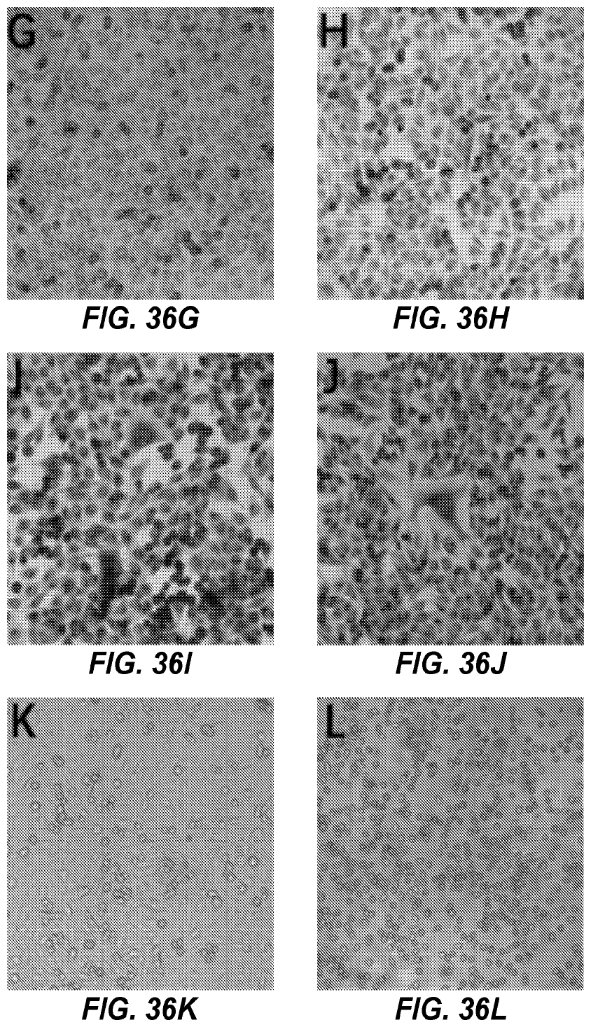

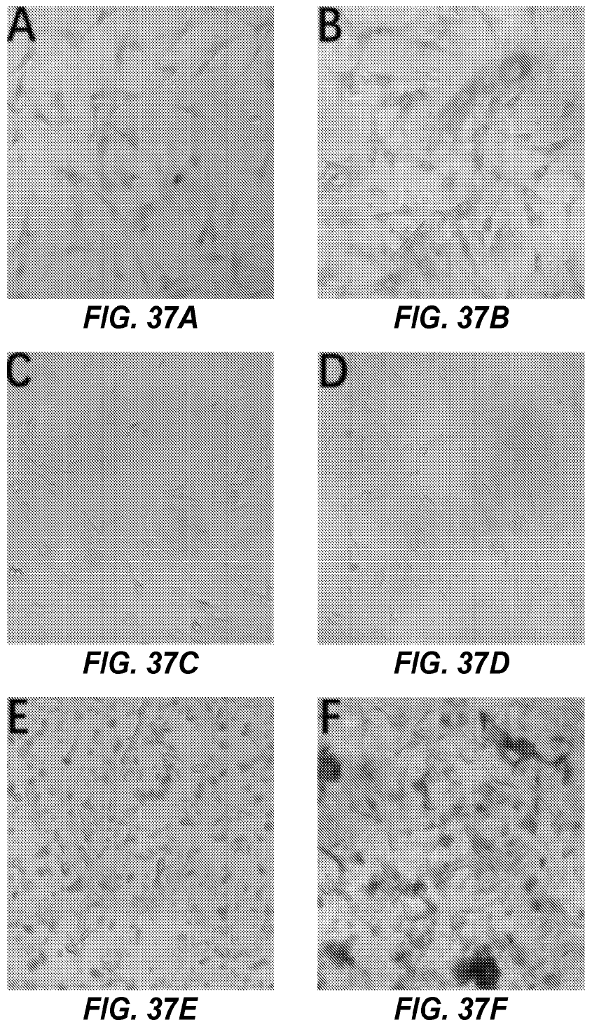

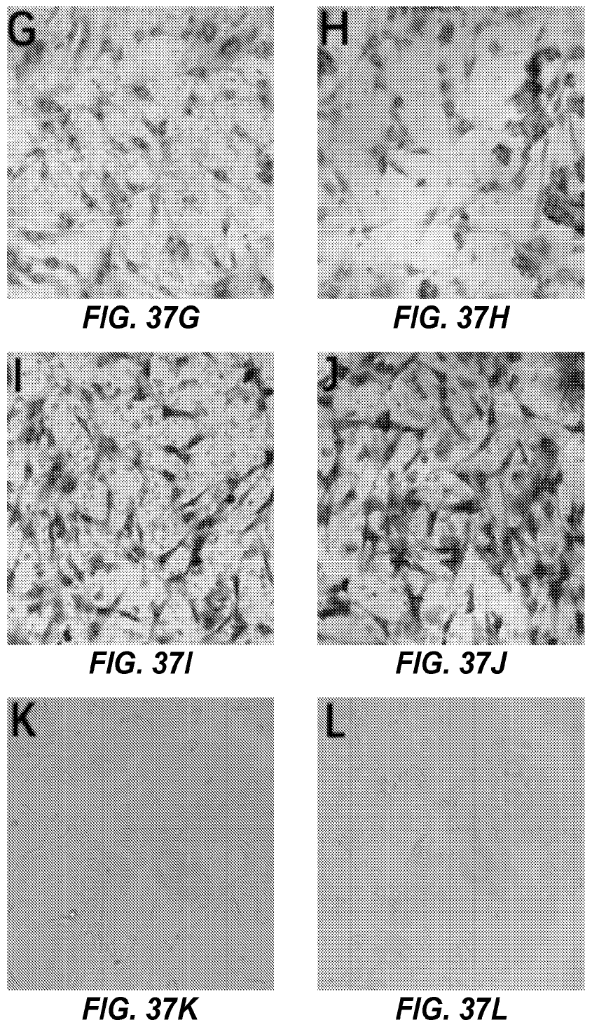

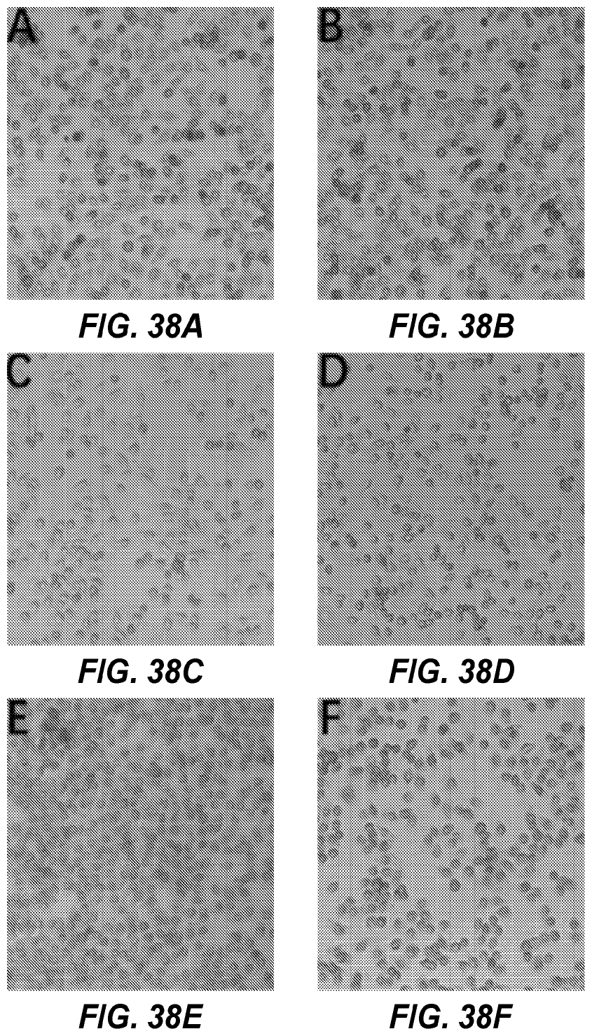

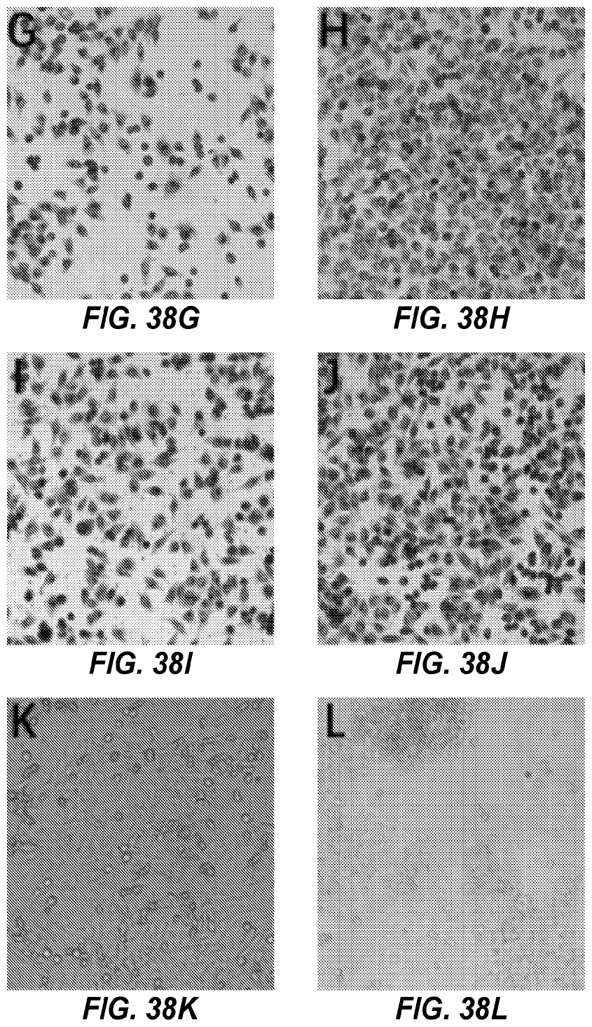

As shown in FIGS. 35A-35L (for HDF cells) and in FIGS. 36A-36L (for melanoma cells), HDF and melanoma cells at 0 (left side) and 24 hours (right side) were studied after inoculation with high temperature conditions. As it can be seen, the cells treated with different metallic salts, Au (A to B), Pt (C to D), Pd (E to F), AuPt (G to H) and AuPd (I to J), kept attached to the bottom and did not lose their morphology as a consequence of the presence of metallic nanoparticles. On the other hand, those untreated cells (K to L) lost their structure as a consequence of cell death due to the high temperature conditions. As shown in FIGS. 37A-37L (for HDF cells) and in FIGS. 38A-38L (for melanoma cells), HDF and melanoma cells at 0 (left side) and 24 hours (right side) were studied after inoculation with low temperature conditions. As it can be seen, the cells treated with different metallic salts, Au (A to B), Pt (C to D), Pd (E to F), AuPt (G to H) and AuPd (I to J), kept attached to the bottom and did not lose their morphology as a consequence of the presence of metallic nanoparticles. On the other hand, those untreated cells (K to L) lost their structure as a consequence of cell death due to the low temperature conditions.

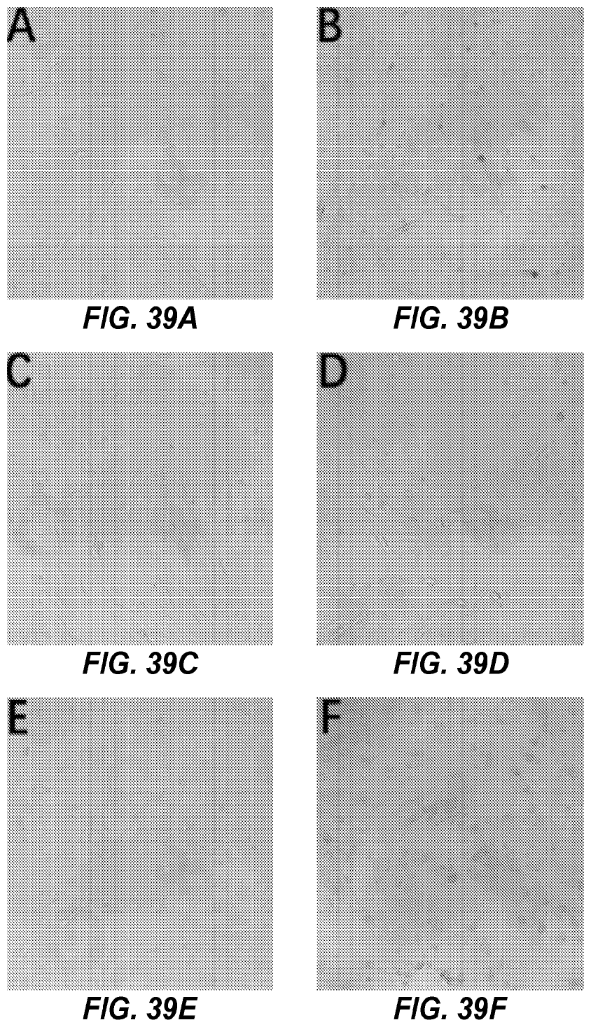

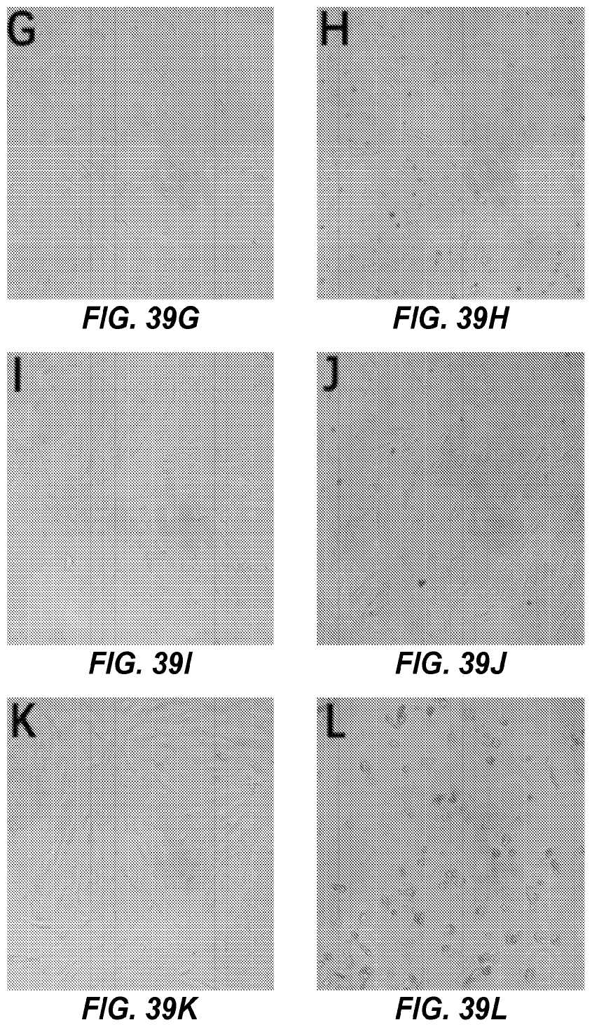

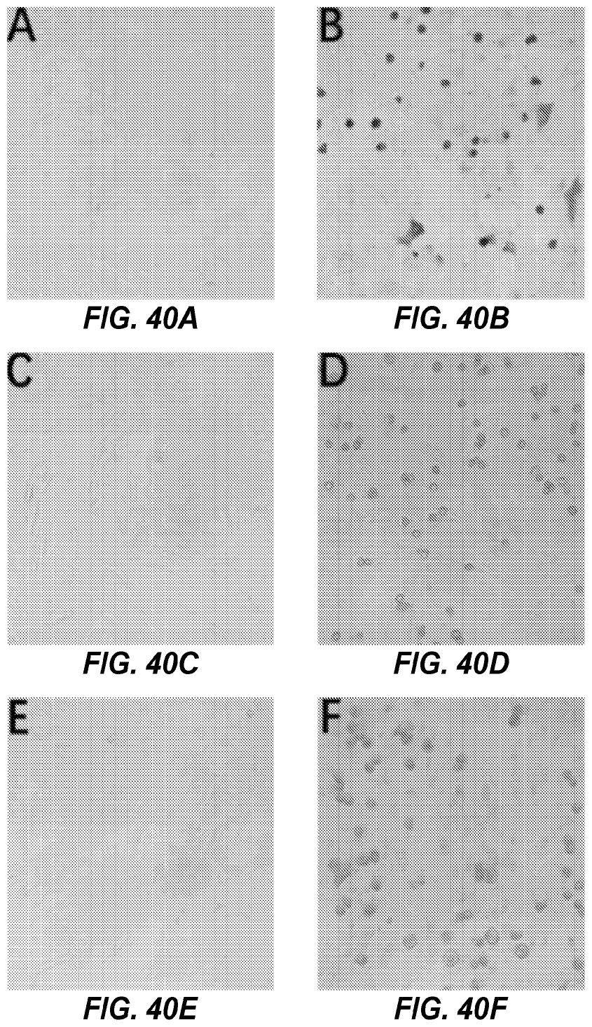

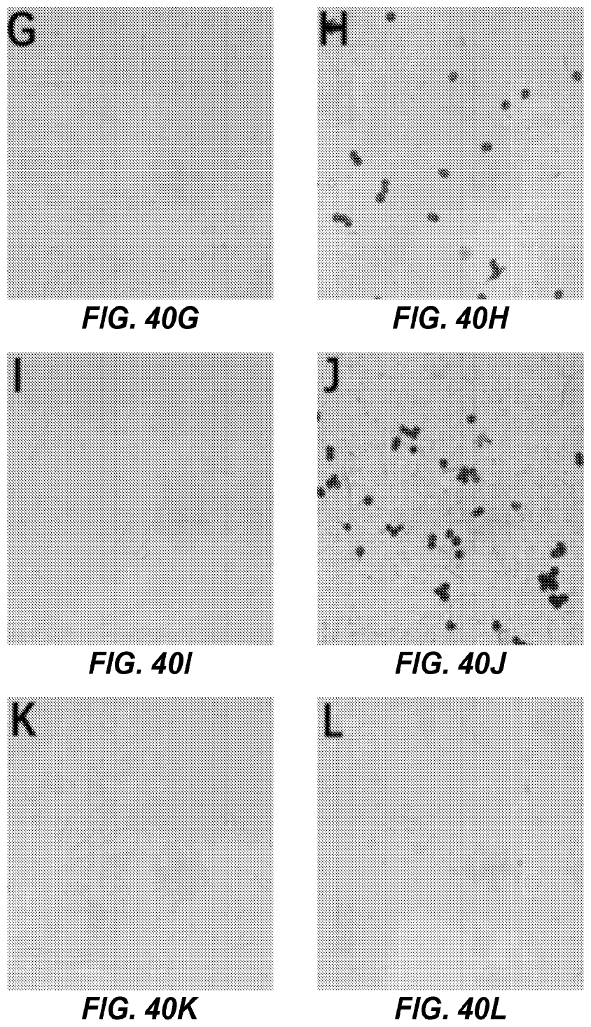

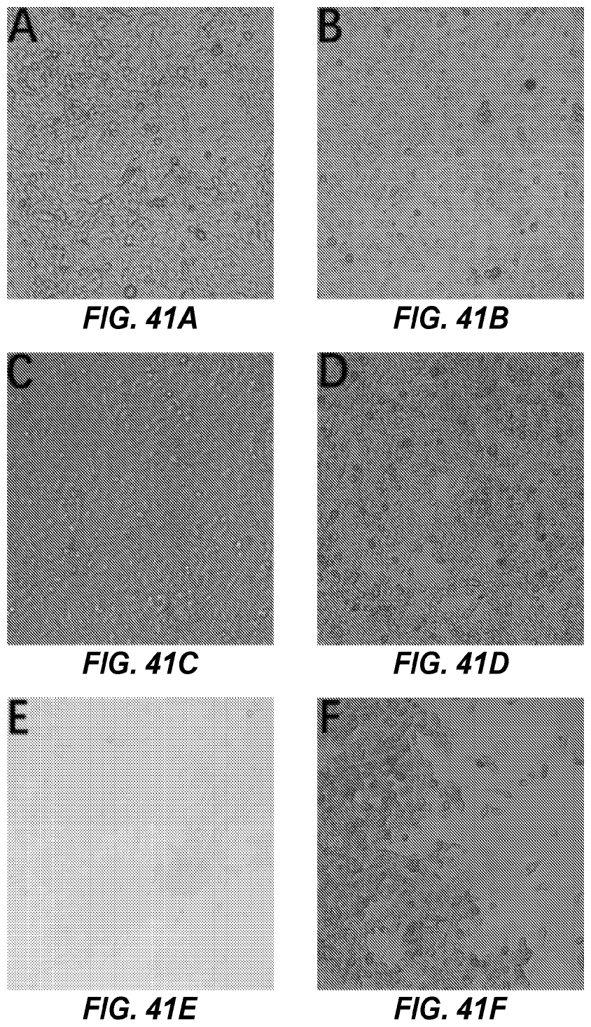

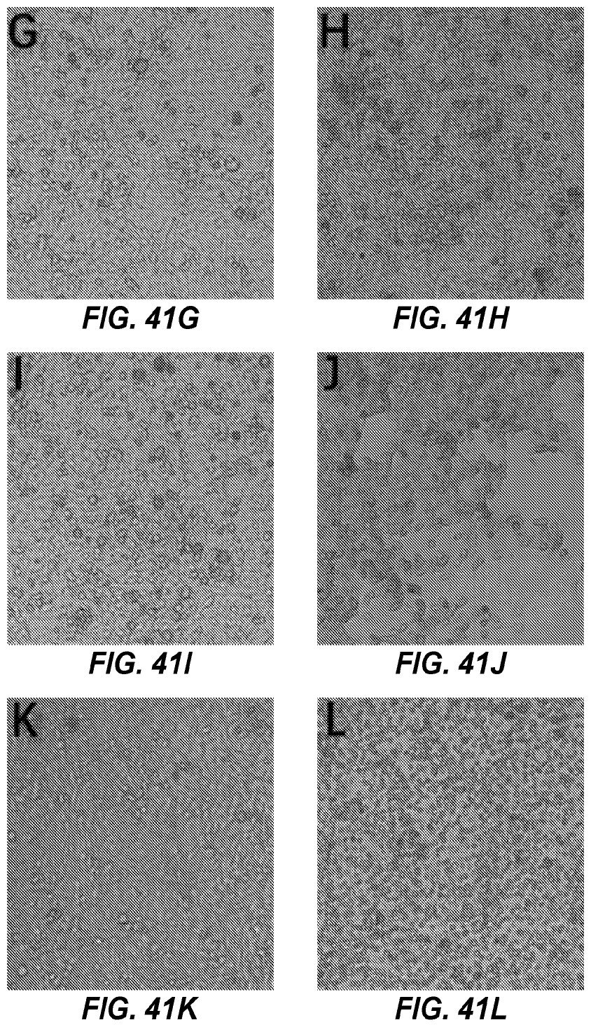

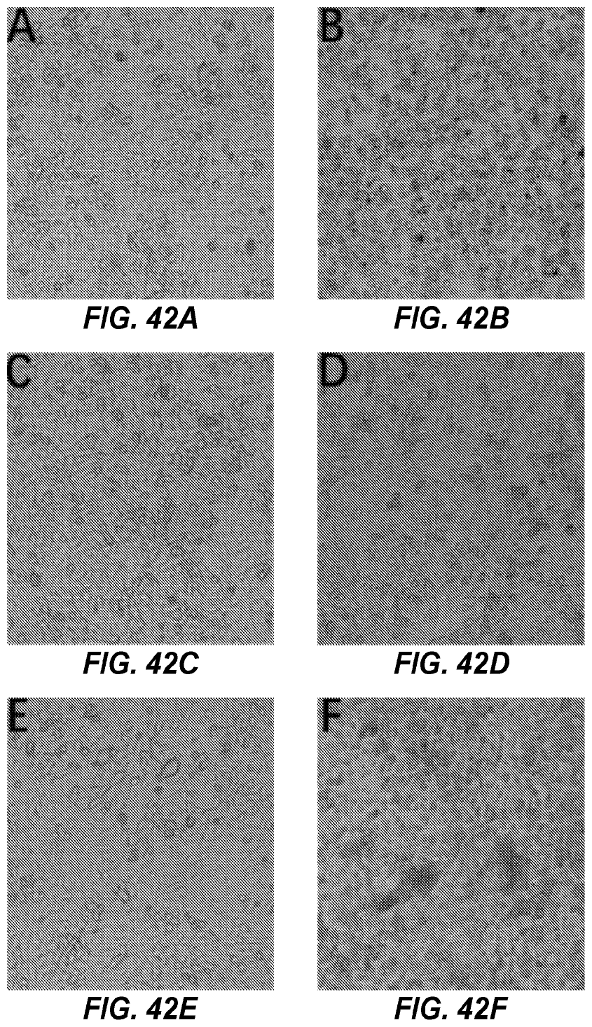

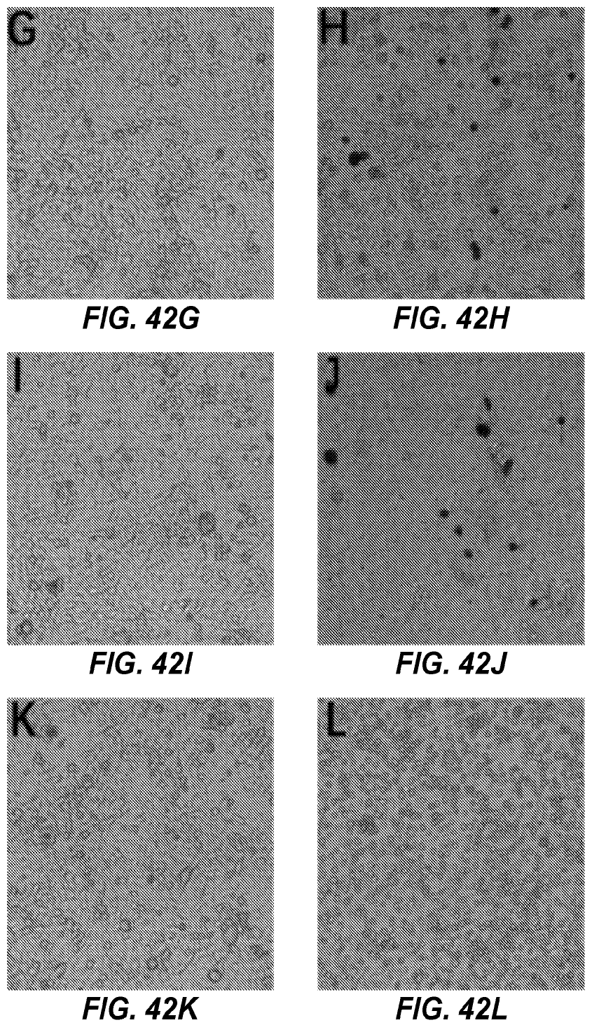

With the aim to observe if the liquid media used for the production of nanoparticles in one experiment contained enough metallic ions to trigger the synthesis of more nanoparticles in a new cell experiment, the liquid media of experiments with HDF and melanoma cells were collected after synthesis and used in completely new experiments. In the first set of experiments, HDF cells were cultured with liquid cell media collected from HDF-NPs (FIGS. 39A-39L) and MEL-NPs (FIGS. 40A-40L) synthesis, while the second set corresponded to melanoma cells cultured with liquid cell media collected from HDF-NPs (FIGS. 41A-41L) and MEL-NPs (FIGS. 42A-42L) synthesis, respectively.

As shown in FIGS. 39A-39L, HDF cells at 0 (left side) and 72 hours (right side) were subjected to changes due to the presence of the liquid media used as a metallic precursor for a (previous) HDF-NPs experiment. As it can be seen, the cells treated with different used metallic salt liquid media, Au (A to B), Pt (C to D), Pd (E to F), AuPt (G to H), and AuPd (I to J) show some differences. The used liquid media, containing metallic ions, was able to trigger the formation of some nanoparticles in these new cells, since they remained attached to the bottom, showing similar results to those explained in the other Examples and synthetic procedures. Therefore, it is possible to hypothesize that the metallic salt concentration added to DPBS is more than enough to allow the synthesis of nanoparticles in, at least, two different experiments, not becoming exhausted.

In FIGS. 40A-40L, HDF cells at 0 (left side) and 72 hours (right side) were subjected to changes due to the presence of the liquid media used as a metallic precursor for a MEL-NPs experiment. As it can be seen, the cells treated with different metallic salts, Au (A to B), Pt (C to D), Pd (E to F), AuPt (G to H), AuPd (I to J), and DPBS (K to L) from melanoma ones remained attached to the bottom and their morphology changed slightly. However, the cluster of death cells appeared, and the cell density decreased. On the other hand, those cells that were treated with DPBS (K to L) lost their structure as a consequence of the extreme conditions.

In FIGS. 41A-41L, melanoma cells at 0 (left side) and 72 hours (right side) were subjected to changes due to the presence of the liquid media used as a metallic precursor an HDF-NPs experiment. The cells treated with different metallic salts, Au (A to B), Pt (C to D), Pd (E to F), AuPt (G to H) and AuPd (I to J) and DPBS (K to L), from HDF ones remained attached to the bottom and their morphology changed slightly. However, as can be seen, the cell density decreased due to the reuse of metallic salt precursor from HDF-NPs experiment. On the other hand, those cells that were treated with DPBS (K to L) lost their structure as a consequence of the extreme conditions.

In FIGS. 42A-42L, melanoma cells at 0 (left side) and 72 hours (right side) were subjected to changes due to the presence of the liquid media used as a metallic precursor a melanoma-NPs experiment. The cells treated with different metallic salts, Au (A to B), Pt (C to D), Pd (E to F), AuPt (G to H) and AuPd (I to J) and DPBS (K to L), from melanoma ones remained attached to the bottom and did not lost their morphology. On the other hand, those cells that were treated with DPBS (K to L) lost their structure as a consequence of the extreme conditions.

Therefore, it can be considered that the metallic salt precursor coming from HDF-NPs synthesis may damage melanoma cells but have no significant effect on HDF cells. Alternatively, the metallic salt precursor proceeding from MEL-NPs synthesis may damage HDF cells but have no significant damage to melanoma cells. This behavior may be explained due to the presence of nanoparticles in the media with a characteristic coating coming from the cells used for the synthesis, which is in relation with the data obtained in MTS assays.

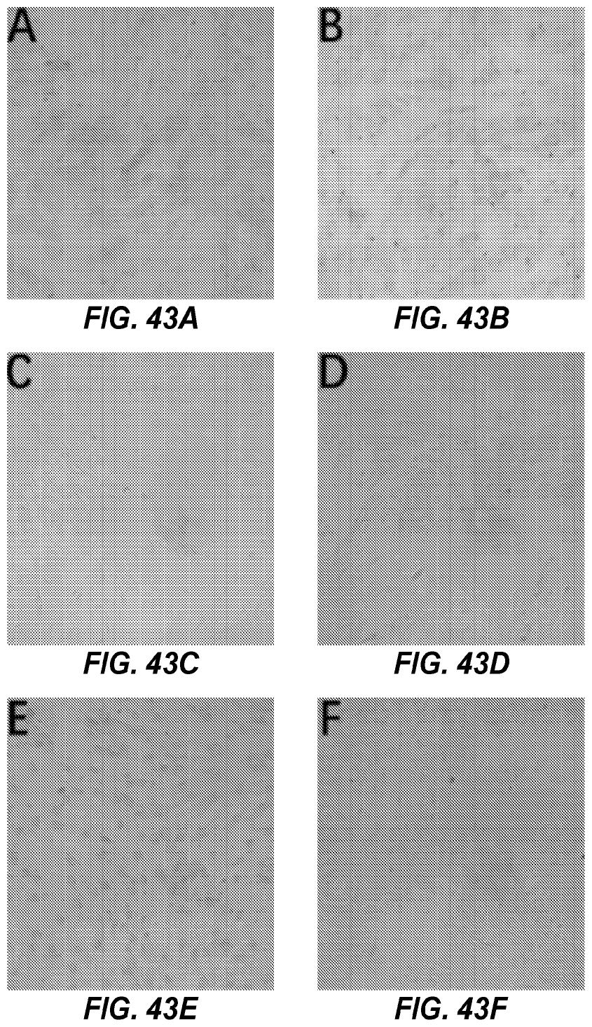

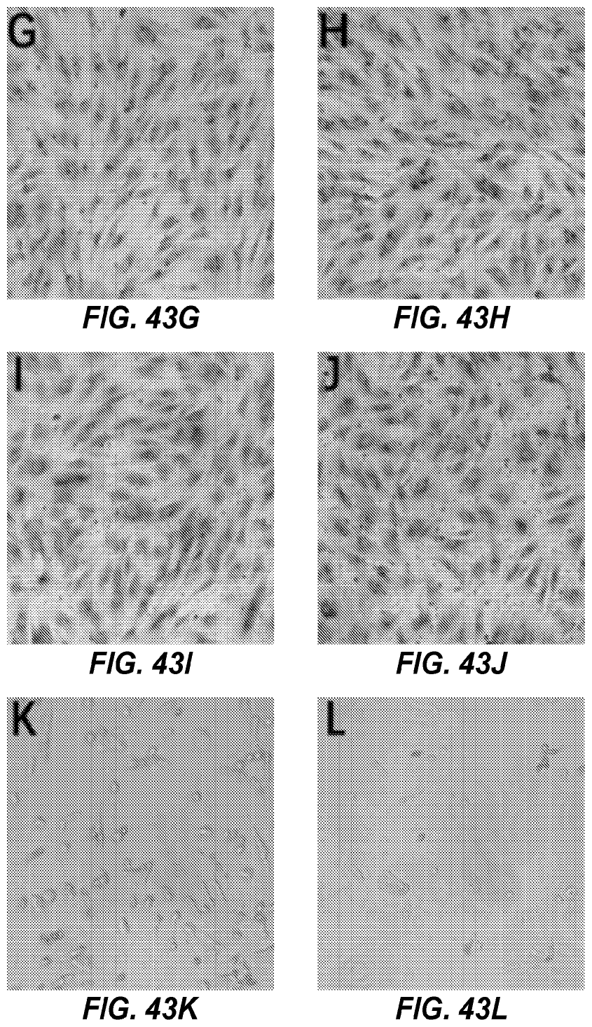

Addition of new media was investigated. The purpose of this experiment was to observe if the cells that were used for the production of nanoparticles were able to proliferate if they were placed in standard conditions with fresh new media. FIGS. 43A-43L show HDF cells previously allowed to produce Au- (A to B), Pd- (C to D), Pt- (E to F), AuPd- (G to H) and AuPt- (I to J) NPs and placed in new media right after addition (left side) and 24 hours after (right side). As can be seen, no apparent proliferation was observed. Besides, HDF cells that were cultured in just DPBS (K to L) and placed back in media did not proliferate as well, as MTS assays were performed to numerically quantify this behavior.

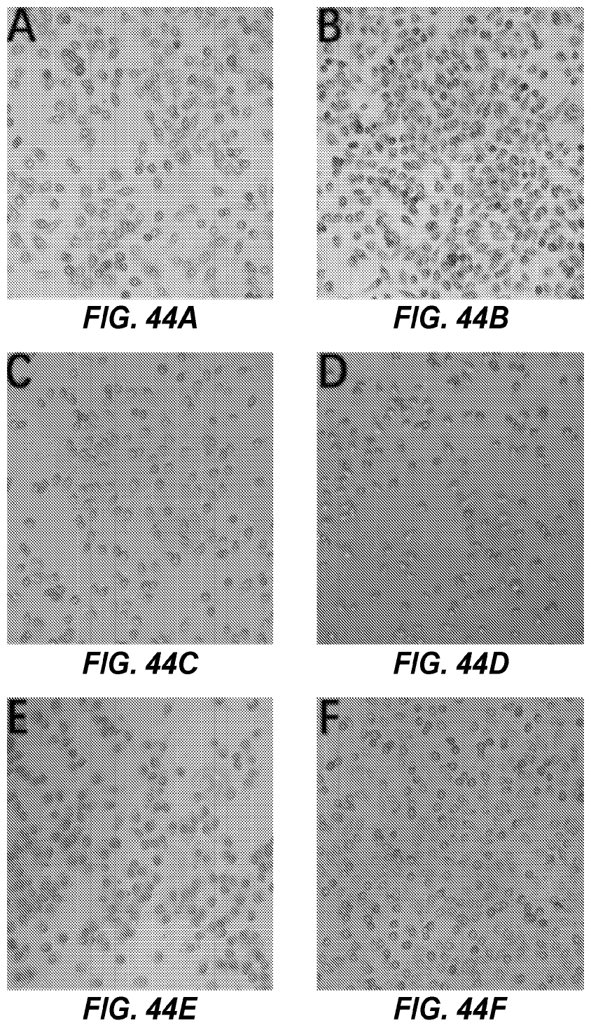

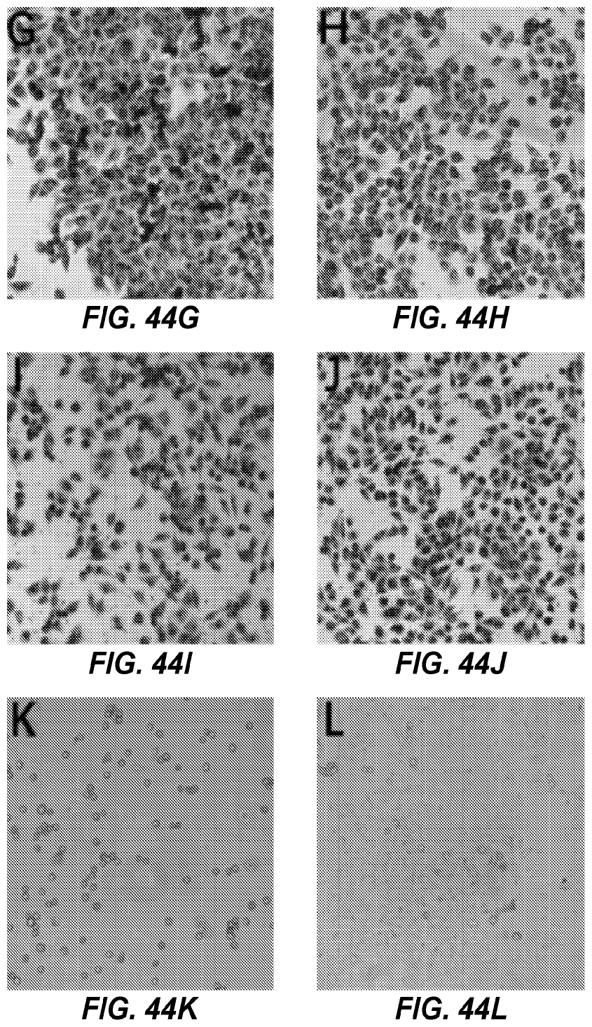

A similar behavior was observed in FIGS. 44A-44L, which shows melanoma cells previously allowed to produce Au- (A to B), Pd- (C to D), Pt- (E to F), AuPd- (G to H) and AuPt- (I to J) NPs and placed in new media right after addition (left side) and 24 hours after (right side). As a can be seen, no apparent proliferation was observed. Moreover, melanoma cells that were cultured in just DPBS (K to L) and placed back in media did not proliferate as well. MTS assays were performed to numerically quantify this behavior.

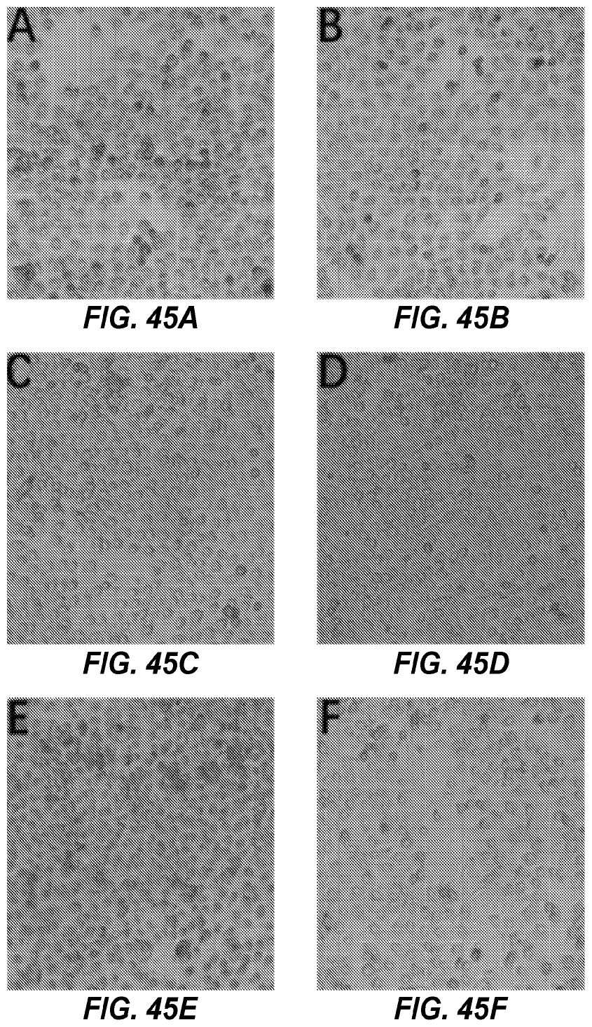

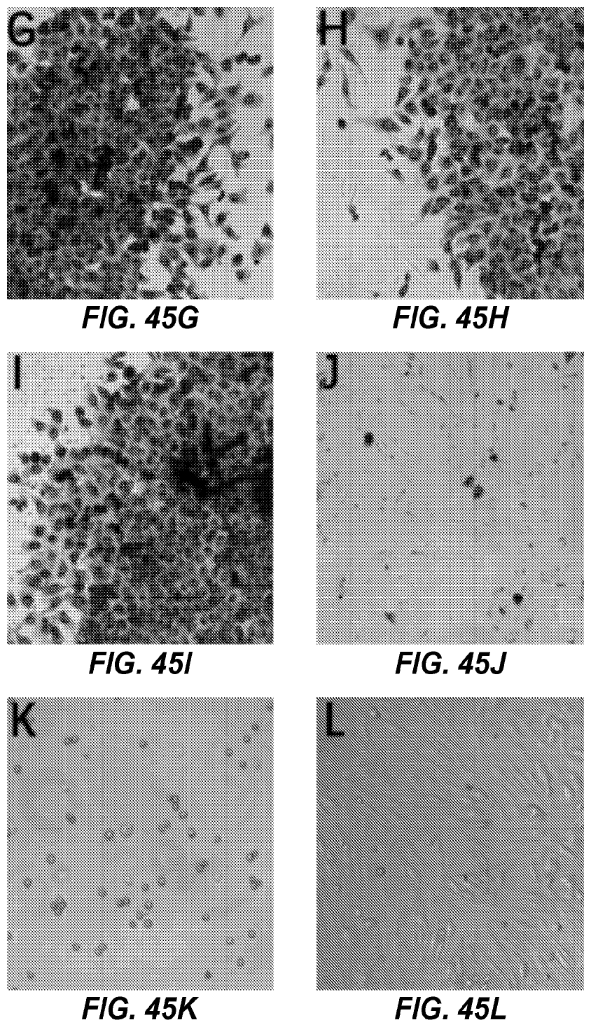

Combinations were studied, for example, a melanoma-HDF cells combination. The experiment purpose is to qualitatively study the possibility of new HDF cells proliferating on top of melanoma cells that were subjected to the production of nanoparticles and whose growth, as a consequence of this, was stopped. FIGS. 45A-45L show melanoma cells previously allowed to produce Au- (A to B), Pd- (C to D), Pt- (E to F), AuPd- (G to H) and AuPt- (I to J) NPs and placed in new media containing HDF cells, right after inoculation (left side) and 24 hours after (right side). It can be seen that HDF cells are able to proliferate together with the static melanoma cells for all the experiments, with a higher density in the control experiment (FIGS. 45K and 45L).

An application of these biologically-synthesize nanostructures is biomedical imaging, which has become an indispensable tool for early, rapid, accurate and cost effective diagnosis of cancer and many other non-cancerous diseases. In the case of cancer, diagnosis before the onset of metastasis is vital to help decrease the mortality rate. Although many nanoscale materials have been reported useful for biomedical applications, including imaging, there are several drawbacks, i.e., poor target recognition, triggering of autoimmune reactions, lower serum albumin binding or the hydrophobic nature of nanoscale particles. Moreover, nanomaterials surface charge is often found as a concern, since the cell membrane is negatively charged and all negatively charged nanomaterials will lead to poor target recognition and prolonged circulation time, which will result in adverse toxic effects. Besides, nanoparticles have to rely on passive cellular uptake to pass the cell membrane and have to escape the endosomal/lysosomal pathway within the cell for the desired effects.

The cell nucleus can be considered the most important cell organelle because it encompasses the genetic information that plays a critical role in most cell functions i.e. cell growth, proliferation, and cell apoptosis. Therefore, targeting the nucleus with nanostructures is a promising approach in biological research due to its role in different cell functions. In addition, nanoparticles targeting of cancer cell nuclei has been reported to influence cellular function, causing cytokinesis arrest, DNA damage, and programmed cell death, which leads to failed cell division, thereby resulting in apoptosis. However, nuclear targeting is difficult to achieve because the nanoparticles must pass into the cytoplasm and then cross the nuclear membrane. Consequently, some studies have attempted to develop methods of forming metal NPs inside the human cell nucleus.

In the UV-Vis spectra reported in FIGS. 1A-2E, all of the lysed (lysis=♦) spectra have higher absorbance compared to the 0 hour, 6 hours, 12 hours, and 24 hours spectra, except for FIG. 2B, in which the lysis spectrum has higher absorbance from about 550 nm to 800 nm. These spectra support that lysis releases nanoparticles. The SEM data presented in FIGS. 11A-14F show nanoparticle growth at the cell membrane. SEM is a surface imaging technique, without penetration underneath the cell membrane, so visualization by SEM of nanoparticles below the cell membrane is not expected in the SEM images.

A method of targeting a cell nucleus can comprise contacting a cell nucleus with a nanoparticle disclosed by the technology herein. The nanoparticles can exhibit passive targeting, wherein size and surface properties will help nanoparticles extravasate through the endothelial wall. The nanoparticles can exhibit active targeting, wherein the nanoparticles will bind to a biomarker of a tissue by a molecular marker or site included. The nanoparticles can include metals, such as gold (Au), palladium (Pd), platinum (Pd), silver (Ag); metalloids, like selenium (Se) or tellurium (Te); oxides, such as zinc oxide (ZnO), copper oxide (CuO); magnetic materials, like iron oxide (Fe₂O₃) or magnetite (Fe₃O₄); and some bimetallic formulations, such as gold-palladium (AuPd), gold-platinum (AuPt), silver-selenium (Ag—Se), and platinum-palladium (Pt— Pd). A method of in vivo bio-imaging or targeting of a cancer cell or of a specific type of cell can comprise contacting a cell with a nanoparticle disclosed by the technology herein.

The technology presents a green, environmentally-friendly, and cost-effective approach for the production of metallic nanoparticles using human cells, that clearly overcomes the main limitations of traditional synthesis in terms of production and biocompatibility and provides extreme benefits for cancer treatments, imaging, and targeting of cells.

### EXAMPLES

### Example 1: Synthesis and Purification

In general, to prepare human cells for synthesis or testing, one vial of HDF or melanoma cells was taken out from cold storage and put in 37° C. water base. After melted, the cells were transferred to a 15 mL Falcon conical centrifuge tube with 5 mL suitable media. Then the tube was centrifuged at 1100 rpm for 5 minutes. The liquid phase was removed, and 5 mL new suitable media was added. Then the cells were well-mixed by gently moving a pipette up and down to form single cell suspension in media. Finally, the cells were transferred in a T-75 cell culture flask with 10 mL suitable media and allowed to grow until 80% confluence.

For different uses of the cells, such as synthesizing nanoparticles and testing cytotoxicity of nanoparticles, the growth medium was rinsed out and the cells were washed once with Dulbecco's Phosphate Buffered Saline (DPBS). Then 3 mL 0.25% Trypsin, 2.21 mM EDTA, 1× was added to the T-75 cell culture flask and incubated for 5 minutes until all the cells were detached. 10 mL of the suitable medium was added to the T-75 cell culture flask, then all the medium with cells were transferred to a 15 mL Falcon conical centrifuge tube. After centrifugation at 1100 rpm for 5 mins, 5 mL of the suitable medium was added to the cell pellet. After mixing the cells gently, the cell concentration was counted using a Hausser Scientific Bright Line™ Counting Chamber under the microscope. Then the cells were seeded in a T-75 cell culture flask, 6, 12, or 96 well plate at the cell density of 2×10⁶ cells/flask, 3×10⁵ cells/well, 1×10⁵ cells/well, and 5×10⁴ cells/well, respectively, in the suitable media and allowed to grow to 80% confluency.

To carry out synthesis and purification, the growth medium was rinsed out and the cells were washed once with DPBS. Then, cells were incubated with 1 mL (12 well plate, Corning®, NY), 2 mL (6 well plate, Corning®, NY) or 14 mL (T-75 Flask, Thermo Fisher Scientific, Waltham, Mass.) of 1.5 mM HAuCl₄ (Gold chloride, Sigma, St. Louis, Mo.), K₂PtCl₄ (Potassium tetrachloroplatinate, Sigma, St. Louis, Mo.), K₂PdCl₄ (Potassium tetrachloropalladate, Sigma, St. Louis, Mo.), HAuCl₄ and K₂PtCl₄, HAuCl₄ and K₂PdCl₄ with DPBS (pH 7.4). Then, the treated cells were kept in the incubator for 1 day at 37° C. and 5% CO₂ atmosphere.

At 24 hours of incubation, flasks were devoted to the preparation of the cell lysate. The cells were scraped off the flask surface using a cell scraper. Thereafter, the cell suspension in the flask was transferred into a centrifuge tube and was sonicated using an ultrasonic homogenizer (model 150VT) with a power source/setting of up to 150 W. This was used for lysis the samples at a duty cycle of 60%. Cell lysis was carried out to ascertain qualitatively the difference in the number of nanoparticles present inside the cytoplasm and in the solution UV-visible spectra of the solution obtained before and after lysis were compared. Then cell lysate was centrifuged at 10,000 rpm for 30 min at 4° C., and the supernatant liquid was separated.

### Example 2: UV-Visible Analysis

Ultraviolet-visible (UV-Vis) characterization was used to follow the progress of the synthesis of nanostructures and the changes within the media in terms of nanoparticles' production. Briefly, 100 μL of each aliquot was taken from the cell solution once after the inoculation with metallic salt was completed, following the reaction up to 24 hours. Aliquots were transferred to a 96-well plate (Falcon clear), and a full absorbance spectrum was recorded from 200 to 800 nm with 10 nm spacing. Near-infrared light can generally refer to 800-2500 nm, so the 200-800 nm absorbance spectrum approached the lower cutoff at the 800 nm NIR range and may be referred to as UV-Vis-NIR.

### Light Microscope

Optical images of the cells were imaged with a Zeiss Axio Observer Z1 inverted microscope once the inoculation with metallic salt was completed, following the reaction of 12, 24, 36, 48 and 72 hours at Pos. 1(Clear aperture), phase 0 and the magnification of 20×. Cell fixation to confirm nanoparticle formation

For the fixation of human dermal fibroblast (HDF) and melanoma cells, the cells were seeded in a 6-well plate with a glass coverslip (Fisher Brand) attached to the bottom. After an incubation period of 24 hours at 37° C. in a humidified incubator with 5% carbon dioxide (CO₂), media was removed and replaced with DPBS containing a concentration of 1.5 mM of metal solutions. Cells were cultured for another 24 hours under the same conditions.

After the experiments, the coverslips were fixed with a primary fixative solution containing 2.5% glutaraldehyde and 0.1 M sodium cacodylate buffer solution for 1 hour. Subsequently, the fixative solution was exchanged for 0.1 M sodium cacodylate buffer and the coverslips were washed 3 times for 10 mins each. Post-fixation was done using 1% osmium tetroxide (OsO₄) solution in the buffer for 1 hour. Subsequently, the coverslips were washed three times with buffer and dehydration was progressively achieved with 35, 50, 70, 80, 95 and 100% ethanol, three times for the 100% ethanol. Finally, the coverslips were dried by liquid CO₂-ethanol exchange in a Samdri®-PVT-3D Critical Point Dryer. The coverslips were mounted on SEM stubs with carbon adhesive tabs (Electron Microscopy Sciences, EMS) after treatment with liquid graphite, and then sputter coated with a thin layer of platinum using a Cressington 208HR High Resolution Sputter Coater. Digital images of the treated and untreated cells were acquired using an SEM. For cell fixation studies, a Cressington 208HR High-Resolution Sputter Coater and a Samdri®-PVT-3D Critical Point Dryer was used to prepare the samples, that were imaged using a Hitachi S-4800 SEM instrument under a 3-kV accelerating voltage and 10 μA of the current condition.

### Example 3: In Vitro Cytotoxicity of Human Cell-Mediated Synthesized Nanoparticles with Healthy and Cancer Cells

Cytotoxicity assays were performed on human dermal fibroblast (ATCC® CCL110™, Manassas, Va.) cells and human melanoma cells (ATCC® CRL1619™, Manassas, Va.). The cells were grown in Eagles Minimum Essential Medium (EMEM, ATCC® 30-2003™, Manassas, Va.) and Dulbecco's Modified Eagle Medium (DMEM; Thermo Fisher Scientific, Waltham, Mass.) respectively, supplemented with 10% fetal bovine serum (FBS; ATCC® 30-2020™, Manassas, Va.) and 1% penicillin/streptomycin (Thermo Fisher Scientific, Waltham, Mass.). Cell viability (MTS) assays (CellTiter 96® AQueous One Solution Cell Proliferation Assay, Promega, Madison, Wis.) were carried out to assess cytotoxicity. Cells were seeded onto tissue-culture treated 96-well plates (Thermo Fisher Scientific, Waltham, Mass.) at a concentration of 50,000 cells per well in 100 μL of medium. After an incubation period of 24 hours at 37° C. in a humidified incubator with a 5% CO₂ atmosphere, the culture medium was aspirated from the wells and replaced with 100 μL of fresh medium containing a defined concentration of nanoparticles. Experimental controls containing medium alone and HDF cells with medium were also prepared. The plate was then incubated for 24 hours and 72 hours under the same environmental conditions. The culture medium was removed and replaced with 100 μL of MTS solution containing a ratio of 1-part MTS to 5-part mediums. After adding the MTS solution, the 96-well plate was incubated for 4 hours at 37° C. to allow for the reduction of MTS to formazan by viable cells. The absorbance was then measured at 490 nm on an absorbance plate reader (SpectraMax Paradigm Multi-Mode Detection Platform, Molecular Devices, Sunnyvale, Calif.), and the cell viability in response to various metal nanoparticle concentrations was determined. Cell viability was calculated by dividing the average absorbance obtained for each sample by the absorbance of the control sample with no nanoparticles, and then multiplying the result by 100 to obtain percent viability.

### Example 4: Cell Fixation for Nanoparticles Against Human Cells

For the fixation nanoparticles made from HDF cells against HDF cells and melanoma cells, the cells were seeded in a 6-well plate with a glass coverslip (Fisher Brand) attached to the bottom. After an incubation period of 24 hours at 37° C. in a humidified incubator with 5% CO₂, media was removed and replaced with different concentration of nanoparticles suitable media. Cells were cultured for another 24 hours at the same conditions.

After the experiments, the coverslips were fixed with a primary fixative solution containing 2.5% glutaraldehyde and 0.1 M sodium cacodylate buffer solution for 1 hour. Subsequently, the fixative solution was exchanged for 0.1 M sodium cacodylate buffer and the coverslips were washed 3 times for 10 min. Post-fixation was done using 1% osmium tetroxide (OsO₄) solution in the buffer for 1 hour. Subsequently, the coverslips were washed three times with buffer and dehydration was progressively achieved with 35, 50, 70, 80, 95 and 100% ethanol, three times for the 100% ethanol. Finally, the coverslips were dried by liquid CO₂-ethanol exchange in a Samdri®-PVT- 3D Critical Point Dryer. The coverslips were mounted on SEM stubs with carbon adhesive tabs (Electron Microscopy Sciences, EMS) after treatment with liquid graphite, and then sputter coated with a thin layer of platinum using a Cressington 208HR High Resolution Sputter Coater. Digital images of the treated and untreated cells were acquired using an SEM. For cell fixation studies, a Cressington 208HR High-Resolution Sputter Coater and a Samdri®-PVT-3D Critical Point dryer was used to prepare the samples, that were imaged using a Hitachi S-4800 SEM instrument was used with a 3-kV accelerating voltage and 10 μA of current.

### Example 5: Reactive Oxygen Species (ROS) Analysis

For ROS quantification, 2′,7′-dichlorodihydrofluorescein diacetate (H₂DCFDA) was used. Human melanoma cells were seed in a 96 well-plate at a concentration of 5×10⁴ cells/mL in the presence of different concentrations of the human cell-mediated nanoparticles as well as in control without any nanoparticles. The cells were cultured under standard culture conditions (37° C. in a humidified incubator with a 5% CO₂ atmosphere) for 24 hours before the experiment. Briefly, the ROS indicator was reconstituted in anhydrous dimethyl sulfoxide (DMSO) to make a concentrated stock solution that was kept and sealed. The growth media were then carefully removed, and a fixed volume of the indicator in DPBS was added to each one of the wells at a final concentration of 10 μM. The cells were incubated for 30 minutes as optimal temperature, and the loading buffer was removed after.

Fresh media were added, and cells were allowed to recover for a short time. The baseline for fluorescence intensity of a sample of the loaded cell period exposure was determined. Positive controls were done stimulating the oxidative activity with hydrogen peroxide to a final concentration of 50 μM. The intensity of fluorescence was then observed by flow cytometry. Measurements were taken by an increase in fluorescence at 530 nm when the sample was excited at 485 nm. Fluorescence was also determined in the negative control, untreated loaded with dye cells maintained in a buffer.

### Example 6: Resistance Study Conditions

In order to assess the response of untreated and treated human cells, HDF and melanoma, with metallic salts to different environmental conditions, a series of experiments to test the resistance of the cells to external stimuli was developed, such a high temperature or an extreme basic pH.

To prepare human cells for testing, the general protocol was followed, one vial of HDF or melanoma cells was taken out from cold storage and put in 37° C. water base. After melted, the cells were transferred to a 15 mL Falcon conical centrifuge tube with 5 mL suitable media. Then the tube was centrifuged at 1100 rpm for 5 minutes. The liquid phase was removed, and 5 mL new suitable media was added. Then the cells were well-mixed by gently moving a pipette up and down to form single cell suspension in media. Finally, the cells were transferred in a T-75 cell culture flask with 10 mL suitable media and allowed to grow until 80% confluence. The growth medium was rinsed out and the cells were washed once with Dulbecco's Phosphate Buffered Saline (DPBS). Then 3 mL 0.25% Trypsin, 2.21 mM EDTA, 1× was added to the T-75 cell culture flask and incubated for 5 minutes until all the cells were detached. 10 mL of the suitable medium was added to the T-75 cell culture flask, then all the medium with cells were transferred to a 15 mL Falcon conical centrifuge tube. After centrifugation at 1100 rpm for 5 mins, 5 mL of the suitable medium was added to the cell pellet. After mixing the cells gently, the cell concentration was counted using a Hausser Scientific Bright Line™ Counting Chamber under the microscope. Then the cells were seeded in a T-75 cell culture flask, 6, 12, or 96 well plate at the cell density of 2×10⁶ cells/flask, 3×10⁵ cells/well, 1×10⁵ cells/well, and 5×10⁴ cells/well, respectively, in the suitable media and allowed to grow to 80% confluency. For the resistance study, cells were prepared following this protocol and seeded in 12-well plates.

The experiments were conducted in parallel with a control, consisting in human cells cultured and growth at standards conditions, with no exposure to metallic salts and no subsequent generation of nanoparticles, and an experimental set of cells that were exposed to metallic salts and able to generate nanoparticles following the same experimental protocol for synthesis. To carry out synthesis and purification, the growth medium was rinsed out and the cells were washed once with DPBS. Then, cells were incubated with 1 mL (12 well plate, Corning®, NY), 2 mL (6 well plate, Corning®, NY) or 14 mL (T-75 Flask, Thermo Fisher Scientific, Waltham, Mass.) of 1.5 mM HAuCl₄ (Gold chloride, Sigma, St. Louis, Mo.), K₂PtCl₄ (Potassium tetrachloroplatinate, Sigma, St. Louis, Mo.), K₂PdCl₄ (Potassium tetrachloropalladate, Sigma, St. Louis, Mo.), HAuCl₄ and K₂PtCl₄, HAuCl₄ and K₂PdCl₄ with DPBS (pH 7.4). Then, the treated cells were kept in the incubator for 1 day at 37° C. and 5% CO₂ atmosphere.

At 24 hours of incubation, flasks were devoted to the preparation of the cell lysate. The cells were scraped off the flask surface using a cell scraper. Thereafter, the cell suspension in the flask was transferred into a centrifuge tube and was sonicated using an ultrasonic homogenizer (model 150VT) with a power source/setting of up to 150 W. This was used for lysis the samples at a duty cycle of 60%. Cell lysis was carried out to ascertain qualitatively the difference in the number of nanoparticles present inside the cytoplasm and in the solution UV-visible spectra of the solution obtained before and after lysis were compared. Then cell lysate was centrifuged at 10,000 rpm for 30 min at 4° C., and the supernatant liquid was separated.

At the end of this process, the liquid phase in both plates was removed and a solution of DPBS was added, allowing the experimental conditions. Therefore, the cells were exposed to the same stimuli and light microscopy was accomplished with the aim to observe differences within the cell population in terms of morphology, structure or proliferation.

### Highly-Acidic Conditions

For the highly-acidic conditions experiment, the liquid phase was removed from the plates, followed by the addition of a highly-acidified DPBS at a pH 1±0.2. Subsequently, the plates were placed inside an incubator at standard conditions. Light microscopy characterization was conducted over the two sets of experiments at 0 and 24 hours.

### Highly-Basic Conditions

For the highly-basic conditions experiment, the liquid phase was removed from the plates, followed by the addition of a highly-basic DPBS at a pH 13±0.2. Subsequently, the plates were placed inside an incubator at standard conditions. Light microscopy characterization was conducted over the two sets of experiments at 0 and 24 hours.

### Salt Supersaturation Conditions

For the salt supersaturation conditions experiment, the liquid phase was removed from the plates, followed by the addition of 1 M sodium chloride (NaCl) in DPBS. Subsequently, the plates were placed inside an incubator at standard conditions. Light microscopy characterization was conducted over the two set of experiments at 0 and 24 hours.

### Aqueous Phase Conditions

For the aqueous phase conditions, the liquid phase was removed from the plates, followed by the addition of autoclaved DI water. Subsequently, the plates were placed inside an incubator at standard conditions. Light microscopy characterization was conducted over the two sets of experiments at 0 and 24 hours.

### Concentrated Trypsin Conditions

For the concentrated trypsin conditions, the liquid phase was removed from the plates, followed by the addition of concentrated Trypsin-0.5% solution. Subsequently, the plates were placed inside an incubator at standard conditions. Light microscopy characterization was conducted over the two sets of experiments at 0 and 24 hours.

### High Temperature Conditions

For the high temperature conditions, the liquid phase was removed from the plates and new DPBS was added free of metallic ions. Subsequently, the plates were placed inside a previously-sterilized oven at 50° C. conditions for 24 hours. Light microscopy characterization was conducted over the two sets of experiments at 0 and 24 hours.

### Low Temperature Conditions

For the low temperature conditions, the liquid phase was removed from the plates and new DPBS was added free of metallic ions. Subsequently, the plates were placed inside a freezer at −80° C. for 24 hours. After that time, the plate was placed in a sterilized surface until the cells reached room temperature. Light microscopy characterization was conducted over the two sets of experiments at 0 and 24 hours after the plates were removed from the freezer.

### Reuse of Metallic Salt Precursor

With the aim to assess the potential of the metallic salt solution/DPBS as a precursor for new nanoparticle formation, the cells were subjected to a synthetic process and right after, the liquid was removed from the plates. This volume was subsequently added to a new plate and the synthetic protocol was started again.

### Addition of New Media

The viability and potential proliferation of cells that were subjected to nanoparticle synthesis were evaluated. After synthesis, the liquid phase was removed from the cells and they were rinsed twice with PBS to remove any metallic ions left. Then, a constant volume of new media was added to the plates, moving the cells from synthetic to standard conditions. Cell viability assays were carried out to assess the proliferation of the cells.

### Melanoma-HDF Cells Combination

Untreated and treated melanoma cells were seeded in the plates. After the synthetic process, the liquid phase was removed, and HDF cells were added in each well at the concentration of 1×10⁵ cells/well, allowing proliferation together with the melanoma cells. The plates were placed inside an incubator at standard conditions. Light microscopy characterization was conducted over the two sets of experiments at different times. Cell viability assays were carried out to assess the proliferation of the cells.

### Example 7: Cell Proliferation Studies

Cell proliferation assays were performed on melanoma cells and HDF cells. The cells were grown in Dulbecco's Modified Eagle Medium (DMEM, Thermo Fisher Scientific, Waltham, Mass.), supplemented with 10% fetal bovine serum (FBS, ATCC® 30-2020™, Manassas, Va.) and 1% penicillin/streptomycin (Thermo Fisher Scientific, Waltham, Mass.). Cell viability (MTS) assays (CellTiter 96® AQueous One Solution Cell Proliferation Assay, Promega, Madison, Wis.) were carried out to assess cell proliferation. Cells were seeded onto tissue-culture treated 96-well plates (Thermo Fisher Scientific, Waltham, Mass.) at a concentration of 50,000 cells per well in 100 μL of the medium. After an incubation period of 24 hours at 37° C. in a humidified incubator with a 5% CO₂ atmosphere, the culture medium was aspirated from the wells and replaced with 100 μL of different solutions, Au, Pd, Pt, AuPd, AuPt salt solutions, and DPBS. After an incubation period of 24 hours at 37° C. in a humidified incubator with a 5% CO₂ atmosphere, the solutions were aspirated from the wells and replaced with 100 μL of fresh medium. Experimental controls containing medium alone and melanoma cells with medium were also prepared. The plate was then incubated for 24 hours and 72 hours under the same environmental conditions. The culture medium was removed and replaced with 100 μL of MTS solution containing a ratio of 1-part MTS to 5-part mediums. After adding the MTS solution, the 96-well plate was incubated for 4 hours at 37° C. to allow for the reduction of MTS to formazan by viable cells. The absorbance was then measured at 490 nm on an absorbance plate reader (SpectraMax Paradigm Multi-Mode Detection Platform, Molecular Devices, Sunnyvale, Calif.), and the cell viability in response to various metal solutions was determined. Cell viability was calculated by dividing the average absorbance obtained for each sample by the absorbance of the control sample with no nanoparticles, and then multiplying the result by 100 to obtain percent viability.

### Stability Analysis and Zeta-Potential

In order to assess the stability of the HDF cells and melanoma cells mediated nanoparticles within time, zeta-potential measurements were carried out in the samples right after synthesis and 60- days or 120-days after this process respectively.

### Statistical Analysis

All experiments were done in triplicate (N=3) to ensure reliability and replicability of results. Experimental results were assessed for statistical significance using a students t-test (p≤0.05 being considered significant). All data were presented as mean±standard deviation.

### Materials and Methods

### Human Cell Lines

Human melanoma cells were acquired (ATCC® CRL-1619™, Manassas, Va.). The cells were grown in Dulbecco's Modified Eagle Medium (DMEM, Thermo Fisher Scientific, Waltham, Mass.), supplemented with 10% fetal bovine serum (FBS, ATCC® 30-2020™, Manassas, Va.) and 1% penicillin/streptomycin (Thermo Fisher Scientific, Waltham, Mass.). While Human dermal fibroblast (ATCC® CCL110™, Manassas, Va.) cells were grown in Eagle's Minimum Essential Medium (EMEM, ATCC® 30-2003™, Manassas, Va.), supplemented with 10% fetal bovine serum (FBS, ATCC® 30-2020™, Manassas, Va.) and 1% penicillin/streptomycin (Thermo Fisher Scientific, Waltham, Mass.). Human dermal fibroblast (HDF) and melanoma (MEL) cell lines were maintained under standard cell culture conditions at 37° C. in an atmosphere of 5% CO₂.

### Instruments and Characterization

A throughout morphological characterization of the nanostructures was accomplished using transmission electron microscopy (TEM) (JEM-1010 TEM (JEOL USA Inc., MA). In order to prepare the samples for imaging, the nanoparticles were dried on 300-mesh copper-coated carbon grids (Electron Microscopy Sciences, Hatfield, Pa.).

Powder XRD patterns were obtained with a Rigaku MiniFlex 600 operating with a voltage of 40 kV, a current of 15 mA, and Cu-Kα radiation (λ=1.542 Å). All XRD patterns were recorded at room temperature with a step width of 0.05 (2θ) and a scan speed of 0.25°/min. The preparation of the sample for XRD analysis was done by drying 2 mL of NPs colloids on the sample holder.

A SpectraMax M3 spectrophotometer (Molecular Devices, Sunnyvale, Calif.) was used to measure the optical density (OD) of the nanoparticle's synthesis process and absorbance in cells.

For cell fixation studies, a Cressington 208HR High-Resolution Sputter Coater and a Samdri®-PVT-3D Critical Point dryer was used to prepare the samples, that were imaged using a Hitachi S-4800 SEM instrument under a 3-kV accelerating voltage and 10 μA of the current condition.

Optical images of the cells were imaged with a Zeiss Axio Observer Z1 inverted microscope. An Eppendorf™ Model 5804-R Centrifuge was used for the centrifugation of samples.

A FreeZone Plus 2.5 Liter Cascade Console Freeze Dry System was used to purify the samples and obtain the final nanoparticles.

An ultrasonic homogenizer (model 150VT) with a power source of up to 150 W was used for lysis of cells and to homogenize the samples.

All frozen cells were stored in CryoPlus™ Storage Systems, while all the live cells were incubated in Thermo Scientific™ CO₂ Incubators except, for example, in the resistance study of high temperature. The cells were incubated in a Benchmark Scientific™ Incu-Shaker.

1. A method of inhibiting the growth of cancer cells in a subject, the method comprising administering a therapeutically effective amount of coated metal nanoparticles to the subject, whereby the growth of the cancer cells in the subject is inhibited;
wherein the metal nanoparticles are produced by a process comprising growing human cells in the presence of a metal salt, whereby metal ions of the salt are reduced to elemental metal to form the metal nanoparticles; whereby the human cells deposit a coating of organic molecules on the metal nanoparticles; and
wherein the coated metal nanoparticles selectively inhibit growth of the cancer cells compared to inhibition by the coated metal nanoparticles of growth of non-cancerous cells in the subject.

2. The method of claim 1, further comprising, prior to said administering:
collecting a sample of the cancer cells and a sample of normal cells from the subject;
cultivating the cancer cells and the normal cells in vitro; and
forming said coated metal nanoparticles by growing the cultivated normal cells in the presence of said metal salt, whereby metal ions of the metal salt are reduced to elemental metal to form said metal nanoparticles.

3. The method of claim 1, wherein the coated metal nanoparticles are at least partially coated with organic molecules provided by the human cells.

4. The method of claim 1, wherein a minimum inhibitory concentration of the coated metal nanoparticles for the cancer cells is in the range from about 5 to 50 μg/mL.

5. The method of claim 1, wherein an IC₅₀ for growth inhibition of the cancer cells is from about 30 to about 65 μg/mL.

6. The method of claim 1, wherein the coated metal nanoparticles have a zeta potential in the range from about 30 mV to about 50 mV.

7. The method of claim 1, wherein the administered coated metal nanoparticles are formulated with one or more pharmaceutically acceptable excipients.

8. The method of claim 1, wherein said coated metal nanoparticles comprise a metal oxide.

9. The method of claim 1, wherein the human cells are selected from human dermal fibroblasts and human melanoma cells

10. The method of claim 1, wherein the coating inhibits the growth of cancer cells.

11. The method of claim 1, wherein the coated metal nanoparticles comprise Au, Ag, Se, Te, ZnO, CuO, Fe₂O₃, Fe₃O₄, Pt, Pd, or a combination thereof.

12. The method of claim 1, wherein the metal salt is selected from the group consisting of HAuCl₄, K₂PtCl₄, K₂PdCl₄, and mixtures thereof.

13. The method of claim 1, wherein the coated metal nanoparticles comprise a radioisotope.

14. The method of claim 1, wherein the coated metal nanoparticles possess a magnetic property.

15. The method of claim 1, wherein the coated metal nanoparticles further comprise a moiety selected from the group consisting of a protein, an antibody, an oligonucleotide, and a small molecule drug.

16. The method of claim 1, wherein the coating is a targeting moiety capable of targeting the coated metal nanoparticles to the cancer cells.

17. The method of claim 1, wherein the cancer cells are cells of a cancer selected from the group consisting of skin cancer, lung cancer, breast cancer, prostate cancer, colorectal cancer, bladder cancer, melanoma, Non-Hodgkin lymphoma, kidney cancer, and leukemia.

18. The method of claim 1, wherein the growth of non-cancerous cells in the subject is not substantially inhibited.

19. The method of claim 1, wherein the therapeutically effective amount provides a concentration of coated metal nanoparticles of about 25 μg/mL at or near the cancer cells.

20. The method of claim 1, wherein the coated metal nanoparticles cause a lethal increase in reactive oxygen species in the cancer cells.

21. The method of claim 1, wherein a portion of the metal nanoparticles is synthesized in the cytoplasm of the human cell.

22. Coated metal nanoparticles produced by a process comprising growing a first type of human cell in the presence of a metal salt, wherein metal ions of the salt are reduced to elemental metal and the first type of human cell deposits a coating of organic molecules on the elemental metal, wherein the coated metal nanoparticles are capable of selectively inhibiting growth of a second type of human cell more than the coated metal nanoparticles inhibit growth of the first type of human cell.

23. The method of claim 22, wherein the coated metal nanoparticles are at least partially coated with organic molecules provided by the first type of human cell during the process of producing the coated metal nanoparticle.

24. The method of claim 22, wherein the organic coating causes the coated metal nanoparticles to selectively inhibit growth of the second type of human cell compared to other types of human cells.

25. The method of claim 22, wherein the organic coating comprises one or more biomolecules specific to the first type of human cells.

26. The method of claim 22, wherein the coated metal nanoparticles further comprise a moiety selected from the group consisting of a radioisotope, a protein, an antibody, an oligonucleotide, a small molecule, and a therapeutic agent.

27. The method of claim 22, wherein the nanoparticles have an average diameter in the range from about 1 nm to about 30 nm, or about 5 to about 25 nm.

28. The method of claim 22, wherein the organic coating is operative to stabilize the coated metal nanoparticles as a colloid or suspension for at least about 60 days.

29. The method of claim 22, wherein the organic coating provides the nanoparticles with a zeta potential exceeding +30 mV which is stable for at least about 60 days.

30. The method of claim 22, wherein the atomic structure of the metal comprises amorphous, FCC, or a combination thereof.

31. The method of claim 22, wherein the coated metal nanoparticles comprise a metal oxide.

32. A method of inhibiting growth of a cancer cell, the method comprising contacting the cancer cell with the coated metal nanoparticles of any of claims 22 to 30, wherein the contacting is performed by administering the coated metal nanostructures to a subject having a cancer, and wherein proliferation of a cancer cell in the subject is inhibited but proliferation of normal cells of the subject is not significantly inhibited.

33. A method of producing coated metal nanoparticles, the method comprising:
(a) contacting a first type of human cell with a metal salt; and
(b) allowing the first type of human cell to reduce the metal salt to elemental metal and to deposit an organic coating on the elemental metal;
whereby coated metal nanoparticles are produced.

34. The method of claim 33, further comprising:
(c) centrifuging the product resulting from step (b) to obtain a pellet;
(d) resuspending the pellet in water; and
(e) lyophilizing the resuspended pellet.

35. The method of claim 33, wherein the resulting coated metal nanoparticles each have a diameter of about 15 nm to about 35 nm.

36. The method of claim 33, wherein the temperature in step (b) is in the range from about 20° C. to about 40° C.

37. The method of claim 33, wherein the atomic structure of the coated metal nanoparticles comprise amorphous metal, FCC metal, or a combination thereof.

38. The method of claim 33, wherein the method produces no byproducts toxic to normal human cells.

39. The method of claim 33, wherein the first type of human cell is a human dermal fibroblast cell or a human melanoma cell.

40. The method of claim 33, wherein the elemental metal or metal oxide is Au, Ag, Se, Te, ZnO, CuO, Fe₂O₃, Fe₃O₄, Pt, Pd, or a combination thereof.

41. The method of claim 33, wherein the metal salt is selected from the group consisting of HAuCl₄, K₂PtCl₄, K₂PdCl₄, and mixtures thereof.
